(19) United States
(12) Patent Application Publication
(10) Pub. No.: US 2022/0227876 A1
(43) Pub. Date: Jul. 21, 2022

(54) RANK ANTAGONISTS AND USES THEREOF

(71) Applicant: The Council of the Queensland Institute of Medical Research, Herston, Queensland (AU)

(72) Inventors: Bill DOUGALL, Herston, Queensland (AU); Elizabeth AHERN, Herston, Queensland (AU)

(21) Appl. No.: 17/311,036

(22) PCT Filed: Dec. 5, 2019
(86) PCT No.: PCT/AU2019/051330

§ 371 Date: Jun. 4, 2021

Related U.S. Application Data
(60) Provisional application No. 62/775,803, filed on Dec. 5, 2018.

(51) Int. Cl. C07K 16/28; A61K 39/395; A61K 38/29; A61K 31/593; A61K 38/18; A61K 45/06; A61P 35/00

(52) U.S. Cl. CPC C07K 16/2878; A61K 39/3955; A61K 38/29; A61K 31/593; A61K 38/1875; A61K 45/06; C07K 2317/626; A61P 35/00; C07K 2317/56; C07K 2317/565; C07K 2317/31; C07K 2317/622; C07K 16/2818

(57) ABSTRACT

Disclosed are antigen-binding molecules that antagonize one or more functions of receptor activator of NF-κB (RANK) as well as methods of their manufacture and use. Applications are also disclosed in which these antagonist antigen-binding molecules are used in compositions and methods for treating or inhibiting the development of conditions associated with activation of the RANK ligand (RANKL)/RANK signaling pathway, for stimulating or augmenting immunity, for inhibiting the development or progression of immunosuppression or tolerance to a tumor and for inhibiting the development, progression or recurrence of cancer.

This application claims priority to U.S. Provisional Application No. 62/775,803 entitled “Antagonists and uses therefor” filed 5 Dec. 2018, the contents of which are incorporated herein by reference in their entirety.

### FIELD OF THE INVENTION

This invention relates generally to antagonist antigen-binding molecules. More particularly, the present invention relates to antigen-binding molecules that antagonize one or more functions of receptor activator of NF-κB (RANK) as well as methods of their manufacture and use. In specific embodiments, the antagonist antigen-binding molecules are used alone or in combination with other agents for treating or inhibiting the development of conditions associated with activation of the RANK ligand (RANKL)/RANK signaling pathway, for stimulating or augmenting immunity, for inhibiting the development or progression of immunosuppression or tolerance to a tumor, or for inhibiting the development, progression or recurrence of cancer.

### BACKGROUND OF THE INVENTION

RANK and RANKL are members of the tumor necrosis factor receptor and ligand superfamilies, respectively, with closest homology to CD40 and CD40L. RANK (TNFRSF11a) and RANKL (TNFSF11) are currently best known in clinical practice for their role in bone homeostasis, as the differentiation of osteoclasts from the monocyte-macrophage lineage requires RANKL interaction with RANK expressed on the myeloid osteoclast precursors (Dougall et al., 1999. Genes Dev. 13(18):2412-2424; Kong et al., 1999. Nature 397(6717):315-323). However, RANKL was initially identified as a dendritic cell-specific survival factor which was upregulated by activated T cells and interacted with RANK on the surface of mature dendritic cells (DCs) to prevent apoptosis (Anderson et al., 1997. Nature 390:175-179; Wong et al., 1997. J. Exp. Med. 186(12):2075-2080). The fully human IgG2 anti-RANKL antibody (denosumab) is widely used in clinical practice as a potent and reasonably well-tolerated anti-resorptive agent for the prevention of skeletal-related events arising from bone metastases, and the management of giant cell tumor of bone and osteoporosis (Branstetter et al., 2012. Clin. Cancer Research 18(16):4415-4424; Fizazi et al., 2011. Lancet (London, England) 377(9768):813-822).

The RANK protein initiates intracellular events by interacting with various TNF Receptor Associated Factors (TRAFs) (Galibert et al., 1998 J Biol Chem 273(51):34120-27). The triggering of RANK, such as by its interaction with RANKL, leads to the multimerization of RANK which recruits TRAFs to the cytoplasmic domain of RANK and activates TRAF-mediated intracellular events, resulting in the upregulation of transcription factors, including NF-KB (Anderson et al., 1997, supra). Signals mediated by the RANK/RANKL interaction are involved in stimulating the differentiation and function of osteoclasts, the cells responsible for bone resorption (see, for example, Lacey et al., 1998. Cell 93:165-7; Yasuda et al., 1998. Proc. Natl. Acad. Sci. USA 95:3597-3602).

RANKL is a key mediator of pathological bone destruction in bone metastases, multiple myeloma, rheumatoid arthritis, wear debris-induced osteolysis, glucocorticoid-induced osteoporosis, osteopenia due to hormone-deprivation therapy, giant cell tumor of bone (GCTB) and postmenopausal osteoporosis (PMO) via stimulation of osteoclast differentiation, activation, and survival (Lacey et al., 1998. Cell 93, 165-176; Boyce and Xing, 2008. Arch. Biochem. Biophys. 473:139-146). The recognition that bone homeostasis was critically regulated by RANKL-RANK signaling prompted the development of denosumab, a fully-human IgG2 monoclonal antibody (MAb) with potent RANKL-neutralizing activity and superior pharmacological properties compared with other RANKL inhibitors (Lacey et al., 2012. Nat. Rev. Drug Discov. 11:401-419). The efficacy of denosumab was subsequently demonstrated in these disease settings and relates to the obligate role of RANKL in the differentiation and functional stimulation of RANK-expressing precursors belonging to the myeloid lineage into bone-resorbing osteoclasts (Dougall et al., 1999, supra).

Recently, the RANK/RANKL system was found to be functionally important in the origin and progression of certain cancers such as breast cancer including of BRCA1-mutation associated breast cancers and hormone-receptor negative and triple negative (ER−, PR−, HER2−) breast cancers (Gonzalez-Suarez et al., 2010. Nature 468(7320):103-107; Nolan et al., 2016. Nat Med 22(8):933-939; Widschwendter et al., 2015, E Bio Medicine 2(10):1331-1339; Pfitzner et al., 2014, Breast Cancer Res Treat. 145(2):307-315; Palafox et al., 2012. Cancer Res. 72(11):2879-2888; Reyes et al., 2017, Breast Cancer Res Treat. 164(1):57-67; Blake et al., 2014. Clin Exp Metastasis 31(2):233-245; Yoldi et al., 2016, Cancer Res. 76(19):5857-5869), prostate cancer (Ohtaka et al., 2017. Int J Surg Case Rep. 30:106-107; Li et al., 2014. Oncol Rep. 32(6):2605-2611), non-small cell lung cancer (NSCLC) including KRAS mutant or KRAS and LKB1 mutant subtypes (Branstetter et al., 2013, Abstract World Conference on Lung Cancer; Rao et al., 2017. Genes Dev. 31, 2099-2112; Faget et al., 2017, J. Thorac. Oncol. 13, 387-398) and renal cell carcinoma (RCC) including clear cell RCC (ccRCC) (Steven et al., 2018. Urol Oncol. 36, 502.e15-502). Accordingly, therapeutic strategies that block RANK/RANKL activity have been proposed for treating these cancers.

Various strategies have been used to develop RANKL/RANK antagonists as therapeutic treatments. For instance, as reviewed in Lacey et al. (2012, supra), several different decoy receptors were developed, which showed different potency as RANKL/RANK antagonists and liabilities, or side-effect profiles. One type of decoy receptor included the chimeric protein encompassing the RANK extracellular domain (ECD) fused to human IgG Fc (RANK-Fc). While this molecule showed promising efficacy in preclinical models, following repeated dosing of human RANK-Fc in non-human primates, activating autoantibody titers against RANK were detected that led to hypercalcemia Lacey et al. (2012, supra). While natural full-length osteoprotegerin (OPG) was demonstrated to be an effective binder and inhibitor of RANKL, development of a therapeutic required testing of hundreds of recombinant variants to improve the pharmacokinetics and bioactivity of these molecules in animals. A recombinant protein containing amino acid residues 22-194 of human OPG fused at the amino terminus to the human immunoglobulin G1 (IgG1) Fc region (Fc-OPG) was tested in Phase 1 clinical trials and demonstrated rapid, dose-related decline in bone turnover markers, indicating that Fc-OPG was a RANKL/RANK antagonist in humans. To improve upon the RANKL/RANK antagonist, an alternative OPG-Fc (AMGN-0007) in which residues 22-194 of human OPG were fused at the carboxyl terminus to human IgG1 Fc expressed in a mammalian cell host (Chinese hamster ovary cells) was demonstrated to have an approximately ten-fold longer half-life and a three- to ten-fold higher potency, compared with Fc-OPG (Lacey et al., 2012, supra).

However, important limitations remain with the application of denosumab as a therapy, including the risk of side effects such as osteonecrosis of the jaw (ONJ), skin rashes, hypocalcemia, or renal toxicity (Prolia package insert; Xgeva package insert). Additionally, serious infections, dermatologic adverse reactions, or atypical bone fractures, the latter perhaps due to ‘frozen bone’, a process in which complete inhibition of osteoclastic bone remodeling leads to accumulation of microfractures and brittle bone, are each toxic consequences of RANKL inhibition using denosumab (Schwarz and Ritchlin, 2007. Res Ther. 9 Suppl 1:S7; Prolia package insert; Xgeva package insert). The efficacy and/or safety of a RANKL/RANK antagonist can be improved by selectively targeting it to the appropriate tissue/cell compartment. For instance, increased distribution of a RANK/RANKL antagonist to the bone, breast, tumor or tumor microenvironment could achieve a greater efficacy and at the same time reduce systemic exposure and associated toxicities. Accordingly, alternative strategies for the development of RANKL/RANK antagonists could provide improved efficacy and safety.

Immunization of the IgG2 XenoMouse strain with human RANKL led to the identification of AMG 162 (a/k/a denosumab), which had high affinity and, importantly, a slow binding off rate to RANKL in equilibrium binding (Lacey et al., 2012, supra). Critically, inhibition of RANKL activity with AMG 162/denosumab was demonstrated in cell-based osteoclast formation assays. While denosumab had a modestly lower affinity for human RANKL compared with recombinant OPG forms, this difference in affinity was more than compensated by the significantly longer circulating half-life of denosumab in vivo, thereby providing substantial efficacy.

Other anti-RANKL antibodies that have been developed include a heavy-chain only (VHH) antibody forms derived from Camillidae, named ALX-0141 (Van de Wetering de Rooij et al., 2011. Ann. Rheum. Dis. 70(3):136; and described in WO2012163887). This anti-RANKL antibody has been assessed in a Phase 1 trial in postmenopausal patients, which indicates a strong and sustained inhibitory effect on bone resorption markers. Furthermore, ALX-0141 was well tolerated and can be administered safely over a wide range of doses.

Other anti-RANKL antibodies or antibody derivatives include Fabs “AT”, “Y”, “P” and “S” derived from a human Fab bacteriophage library (EP1257648). Anti-RANKL Fabs “AT”, “Y”, “P” were demonstrated to antagonize RANKL/RANK using a cell-based osteoclast assay. Other anti-RANKL antibodies include 16E1, 2D8, 2E11, 1862, 2263, or 9H7 generated by immunization of HuMab transgenic mouse strains HCo7, HCo12, and HCo7+HCo12 with purified recombinant RANKL derived from Escherichia coli or Chinese hamster ovary (CHO) cells as antigen (U.S. Pat. No. 8,455,629). Further anti-RANKL antibodies include XPA12.004, XPA12.020, XPA12.039, XPA12.041 and XPA12.042, which were demonstrated to antagonize RANKL/RANK using a cell-based osteoclast assay (WO2011017294).

Other strategies for RANKL/RANK antagonists include formulated siRNAs targeting RANKL, which demonstrated promising results in the treatment of tumor-associated osteolysis (Rousseau et al., 2011. J Bone Miner Res. 26(10):2452-2462).

Another therapeutic alternative is the use of inhibitory peptides, peptidomimetics of protein-protein interaction or antibodies which would block RANKL interaction with RANK or alter the conformation of RANK to reduce its activity and subsequent biochemical signal transduction. For instance, rationally designed small molecule mimics of OPG (“receptor”) or RANKL (“ligand”) were tested in osteoclastogenesis assays in vitro (Cheng et al., 2004. J Biol Chem. 2004; 279(9):8269-8277). Interestingly, peptides designed from OPG showed a greater inhibitory effect than those designed from RANKL, suggesting that receptor-derived mimetics block ligand binding to its receptor differently than ligand mimetics. One OPG mimic (0P3-4) was shown to bind RANKL and RANK, reduced RANKL binding to RANK and inhibited osteoclast formation in vitro and in vivo, thereby functioning as a RANKL/RANK antagonist. One potential mechanism for this antagonism was via alteration in the RANK/RANKL receptor complex, OP3-4 may mediate defective complex either by altering receptor orientation or serving as a “spacer” to prevent cytoplasmic domain interactions, resulting in reduced downstream signaling.

A library of random peptides of variable length was screened for receptor binding against RANK in order to identify RANKL/RANK antagonists (Téletchéa et al., 2014. J Bone Miner Res. 29(6):1466-1477). These experiments demonstrated that two peptides, Pep501 and Pep8, exhibited strong activity in a cell-based osteoclastogenesis assay. Aoki et al. (2006, J Clin Invest. 116(6):1525-1534) reported a cyclic peptide designed to mimic the CRD3 ligand contact surface of the TNFR that binds to TNF; they demonstrated that this WP9QY peptide also inhibits RANKL-induced signaling. While peptide WP9QY inhibits RANKL-induced signaling it did not block the binding of RANKL to RANK. To explain this apparent discrepancy, molecular modeling predicted that peptide WP9QY localized in the binding site for RANK CRD3 would potentially interfere with the proposed ligand-induced clustering of the receptor cytoplasmic domains, thereby functioning as a RANKL/RANK antagonist.

Given the potential for unintended receptor agonism using antagonistic anti-RANK antibodies, antibodies targeting RANKL have been preferred (Lacey et al., 2012, supra). However, using phage display technology, a single chain Fv (scFv) antibody against RANK ECD was identified (Newa et al., 2014. Mol Pharm. 11(1):81-89). Furthermore, anti-RANK scFv blocked RANKL-dependent osteoclast formation activity in the (mouse) RAW264.7 assay. However, whether anti-RANK scFv affected RANKL binding to RANK or, alternatively, altered the RANK receptor complex and downstream signal transduction was not clarified. Subsequently, Chypre et al. (2016, Immunol Lett. 171:5-14) engineered the anti-RANK scFv (now renamed RANK-02) by inserting a missing codon at Kabat position 82 and expressed on human IgG1 heavy and light chain backbone and compared binding characteristics as well as in vitro and in vivo assays to address agonistic vs antagonistic qualities. The ability of RANK-02 to block RANKL was confirmed in ELISA, but when activity of antibodies was tested in a Jurkat huRANK:Fas assay, RANK-02 disappointedly demonstrated agonistic activity. In vivo testing indicated that RANK-02 neither blocked nor potentiated the RANKL-dependent increase in osteoclast TRAP formation. These data indicate that neither binding of antibody to RANK ECD nor ability to block RANKL in vitro predicts antagonistic activity in cell-based or in vivo assays.

### SUMMARY OF THE INVENTION

The present invention is predicated in part on the development of antigen-binding molecules that bind to RANK and antagonize the RANKL/RANK signaling pathway. These antagonist antigen-binding molecules are useful either alone, or in combination with other agents, for treating or inhibiting the development of conditions associated with activation of the RANKL/RANK signaling pathway, for stimulating or augmenting immunity, for inhibiting the development or progression of immunosuppression or tolerance to a tumor, or for inhibiting the development, progression or recurrence of cancer, as described hereafter.

Accordingly, in one aspect, the present invention provides antigen-binding molecules that suitably bind to RANK and antagonize the RANKL/RANK signaling pathway. These antigen-binding molecules generally comprise:
(1) a heavy chain variable region (V_{H}) comprising a VHCDR1 amino acid sequence set forth in SEQ ID NO:3, a VHCDR2 amino acid sequence set forth in SEQ ID NO:4, and a VHCDR3 amino acid sequence set forth in SEQ ID NO:5, and a light chain variable region (V_{L}) comprising a VLCDR1 amino acid sequence set forth in SEQ ID NO:6, a VLCDR2 amino acid sequence set forth in SEQ ID NO:7, and a VLCDR3 amino acid sequence set forth in SEQ ID NO:8;
(2) a V_{H} that comprises the amino acid sequence set forth in SEQ ID NO:1, and a V_{L} that comprises the amino acid sequence set forth in SEQ ID NO:2;
(3) a V_{H} with at least 90% (including at least 91% to 99% and all integer percentages therebetween) sequence identity to the amino acid sequence of SEQ ID NO:1, and a V_{L} with at least 90% (including at least 91% to 99% and all integer percentages therebetween) sequence identity to the amino acid sequence of SEQ ID NO:2;
(4) a V_{H} with at least 90% (including at least 91% to 99% and all integer percentages therebetween) sequence identity to the amino acid sequence of a framework region other than each CDR in the amino acid sequence of SEQ ID NO:1, and a V_{L} with at least 90% (including at least 91% to 99% and all integer percentages therebetween) sequence identity to the amino acid sequence of a framework region other than each CDR in the amino acid sequence of SEQ ID NO:2; or
(5) a V_{H} that comprises an amino acid sequence comprising a deletion, substitution or addition of one or more (e.g., 1, 2, 3, 4 or 5) amino acids in the sequence of a framework region other than at each CDR in the amino acid sequence of SEQ ID NO:1, and a V_{L} that comprises an amino acid sequence comprising a deletion, substitution or addition of one or more (e.g., 1, 2, 3, 4 or 5) amino acids in the sequence of a framework region other than at each CDR in the amino acid sequence of SEQ ID NO:2.

The antigen-binding molecules may be in isolated, purified, synthetic or recombinant form. In specific embodiments, the antigen binding molecules are monovalent antigen-binding molecules (e.g., Fab, scFab, Fab′, scFv, one-armed antibodies, etc.).

The antigen-binding molecules suitably comprise any one or more of the following activities: (a) inhibits binding of RANKL to RANK; (b) inhibits RANK activation; (c) inhibits downstream RANK-mediated molecular signaling (e.g., RANK recruitment of TRAF proteins); (d) inhibits RANK multimerization; (e) reduces osteoclast differentiation; (f) decreases osteoclast activation; (g) reduces osteoclast survival; (h) inhibits bone loss and increase bone density; (i) inhibits immunosuppressive activity of myeloid cells or other immune cells in a tumor microenvironment (TME); and (j) inhibits proliferation, migration, survival and/or morphogenesis of tumor cells (e.g., breast cancer cells including hormone-receptor negative (e.g., ER−; PR−; HER2−; ER−, PR−; ER−, HER2−; PR−, HER2−; and ER−, PR−, HER2−) breast cancer cells, including triple negative breast cancer (TNBC) cells, and/or BRCA-1 mutation positive breast cancer cells, prostate cancer cells, NSCLC cells including KRAS mutant or KRAS and LKB1 mutant NSCLC tumor subtypes, and RCC cells including ccRCC cells).

In some embodiments, the RANK antagonist antigen-binding molecule is contained in a delivery vehicle (e.g., a liposome, a nanoparticle, a microparticle, a dendrimer or a cyclodextrin).

Another aspect of the present invention provides isolated polynucleotides comprising a nucleic acid sequence encoding a RANK antagonist antigen-binding molecule described herein.

Yet another aspect of the present invention provides constructs comprising a nucleic acid sequence encoding a RANK antagonist antigen-binding molecule described herein in operable connection with one or more control sequences. Suitable constructs are preferably in the form of an expression construct, representative examples of which include plasmids, cosmids, phages, and viruses.

In another aspect, the invention provides host cells that contain constructs comprising a nucleic acid sequence encoding a RANK antagonist antigen-binding molecule described herein in operable connection with one or more control sequences.

Yet another aspect of the present invention provides pharmaceutical compositions comprising a RANK antagonist antigen-binding molecule described herein and a pharmaceutically acceptable carrier. In some embodiments, the compositions further comprise at least one ancillary agent selected from a bone anti-resorptive agent (e.g., anabolism enhancers, in particular selected from the group consisting of parathyroid hormone, BMP2, vitamin D, anti-inflammatory agents; and catabolism inhibitors, in particular selected from the group consisting of bisphosphonates, cathepsin K inhibitors, p38 inhibitors, JNK inhibitors, IKK inhibitors, NF-κB inhibitors, calcineurin inhibitors, NFAT inhibitors, PI3K inhibitors) and a chemotherapeutic agent (e.g., antiproliferative/antineoplastic drugs, cytostatic agents, agents that inhibit cancer cell invasion, inhibitors of growth factor function, anti-angiogenic agents, vascular damaging agents, etc.) or an immunotherapeutic agent (e.g., cytokines, cytokine-expressing cells, antibodies, etc.).

A further aspect of the present invention provides methods for inhibiting binding of RANKL to a RANK-expressing cell. These methods generally comprise contacting the RANK-expressing cell with a RANK antagonist antigen-binding molecule described herein, to thereby inhibit binding of RANK to the RANK expressing cell.

In a related aspect, the present invention provides methods for inhibiting activation of RANK on a RANK-expressing cell. These methods generally comprise contacting the RANK-expressing cell with a RANK antagonist antigen-binding molecule described herein, to thereby inhibit activation of RANK on the RANK expressing cell.

In another related aspect, the present invention provides methods for inhibiting RANK-mediated molecular signaling (e.g., RANK recruitment of TRAF proteins) in a RANK-expressing cell. These methods generally comprise contacting the RANK-expressing cell with a RANK antagonist antigen-binding molecule described herein, to thereby inhibit RANK-mediated molecular signaling in the RANK expressing cell.

In yet another related aspect, the present invention provides methods for inhibiting RANK multimerization in a RANK-expressing cell. These methods generally comprise contacting the RANK-expressing cell with a RANK antagonist antigen-binding molecule described herein, to thereby inhibit RANK multimerization in the RANK expressing cell.

Representative RANK-expressing cells include osteoclasts, immune cells such as antigen-presenting cells (e.g., monocytes and dendritic cells) and effector immune cells (e.g., T cells), hematopoietic precursors, and tumor cells (e.g., breast cancer cells including hormone-receptor (HR) negative (e.g., ER−; PR−; HER2−; ER−, PR−; ER−, HER2−; PR−, HER2−; and ER−, PR−, HER2−) breast cancer cells, including triple negative breast cancer (TNBC) cells, and/or BRCA-1 mutation positive breast cancer cells, prostate cancer cells, NSCLC cells including KRAS mutant or KRAS and LKB1 mutant NSCLC tumor subtypes, and RCC cells including ccRCC cells).

In yet another related aspect, the present invention provides methods for inhibiting differentiation, activation and/or survival of an osteoclast. These methods generally comprise contacting the osteoclast with a RANK antagonist antigen-binding molecule described herein, to thereby inhibit differentiation, activation and/or survival of the osteoclast.

In another related aspect, the present invention provides methods for inhibiting immunosuppressive activity of an immune cell (e.g., a myeloid cell or Treg). These methods generally comprise contacting the immune cell with a RANK antagonist antigen-binding molecule described herein, to thereby inhibit the immunosuppressive activity of the immune cell.

In still another related aspect, the present invention provides methods for inhibiting proliferation, survival or migration of a tumor cell. These methods generally comprise contacting the tumor cell with a RANK antagonist antigen-binding molecule described herein, to thereby inhibit proliferation, survival or migration the tumor cell.

Still another aspect of the present invention provides methods for treating or inhibiting the development of a condition associated with activation of the RANKL/RANK signaling pathway in a subject. These methods generally comprise administering to the subject an effective amount of a RANK antagonist antigen-binding molecule described herein, thereby treating or inhibiting the development of the condition. In specific embodiments, the condition associated with RANKL/RANK signaling pathway activation is selected from an osteopenic disorder, a myopathy and a cancer.

In a related aspect, the present invention provides methods for treating or inhibiting the development of bone loss in a subject. These methods generally comprise administering to the subject an effective amount of a RANK antagonist antigen-binding molecule described herein, thereby treating or inhibiting the development of bone loss.

In another related aspect, the present invention provides methods for treating or inhibiting the development of a cancer in a subject, wherein the cancer is associated with activation of the RANKL/RANK signaling pathway. These methods generally comprise administering to the subject an effective amount of a RANK antagonist antigen-binding molecule described herein, thereby treating or inhibiting the development of the cancer. In specific embodiments, the cancer is selected from breast cancer including HR negative (e.g., ER−; PR−; HER2−; ER−, PR−; ER−, HER2−; PR−, HER2−; and ER−, PR−, HER2−) breast cancer, BRCA-1 mutation positive breast cancer, HR negative (e.g., ER−; PR−; HER2−; ER−, PR−; ER−, HER2−; PR−, HER2−; and ER−, PR−, HER2−) and BRCA-1 mutation positive breast cancer, prostate cancer, NSCLC including KRAS mutant or KRAS and LKB1 mutant NSCLC, and RCC cells including ccRCC.

The present inventors have disclosed in co-pending International Application No. PCT/AU2018/050557 filed 5 Jun. 2018, the contents of which are incorporated herein by reference in their entirety, that co-antagonizing RANKL/RANK and an immune checkpoint molecule (ICM) results in a synergistic enhancement in the immune response to a cancer.

Accordingly, in another aspect, the present invention provides a therapeutic combination comprising, consisting, or consisting essentially of a RANK antagonist antigen-binding molecule described herein and at least one anti-ICM antigen-binding molecule. The therapeutic combination may be in the form of a single composition (e.g., a mixture) comprising each of the RANK antagonist antigen-binding molecule and the at least one anti-ICM antigen-binding molecule. Alternatively, the RANK antagonist antigen-binding molecule and the at least one anti-ICM antigen-binding molecule may be provided as discrete components in separate compositions.

The at least one anti-ICM antigen-binding molecule suitably antagonizes an ICM selected from the group consisting of: programmed death 1 receptor (PD-1), programmed death ligand 1 (PD-L1), programmed death ligand 2 (PD-L2), cytotoxic T-lymphocyte-associated antigen 4 (CTLA-4), A2A adenosine receptor (AZAR), A2B adenosine receptor (A2BR), B7-H3 (CD276), V-set domain-containing T-cell activation inhibitor 1 (VTCN1), B- and T-lymphocyte attenuator (BTLA), indoleamine 2,3-dioxygenase (IDO), killer-cell immunoglobulin-like receptor (KIR), lymphocyte activation gene-3 (LAG3), T cell immunoglobulin domain and mucin domain 3 (TIM-3), V-domain Ig suppressor of T cell activation (VISTA), 5′-nucleotidase (CD73), tactile (CD96), poliovirus receptor (CD155), DNAX Accessory Molecule-1 (DNAM-1), poliovirus receptor-related 2 (CD112), cytotoxic and regulatory T-cell molecule (CRTAM), tumor necrosis factor receptor superfamily member 4 (TNFRS4; OX40; CD134), tumor necrosis factor (ligand) superfamily, member 4 (TNFSF4; OX40 ligand (OX40L), natural killer cell receptor 264 (CD244), CD160, glucocorticoid-induced TNFR-related protein (GITR), glucocorticoid-induced TNFR-related protein ligand (GITRL), inducible co-stimulator (ICOS), galectin 9 (GAL-9), 4-1BB ligand (4-1BBL; CD137L), 4-1BB (4-113B; CD137), CD70 (CD27 ligand (CD27L)), CD28, 67-1 (CD80), 67-2 (CD86), signal-regulatory protein (SIRP-1), integrin associated protein (IAP; CD47); B-lymphocyte activation marker (BLAST-1; CD48), natural killer cell receptor 264 (CD244); CD40, CD40 ligand (CD40L), herpesvirus entry mediator (HVEM), transmembrane and immunoglobulin domain containing 2 (TMIGD2), HERV-H LTR-associating 2 (HHLA2), vascular endothelial growth inhibitor (VEGI), tumor necrosis factor receptor superfamily member 25 (TNFRS25), inducible T-cell co-stimulator ligand (ICOLG; B7RP1) and T cell immunoreceptor with Ig and ITIM (immunoreceptor tyrosine-based inhibition motif) domains (TIGIT). In some embodiments, the at least one anti-ICM antigen-binding molecule is selected from a PD-1 antagonist antigen-binding molecule, a PD-L1 antagonist antigen-binding molecule and a CTLA4 antagonist antigen-binding molecule. In some embodiments, the at least one anti-ICM antigen-binding molecule comprises a PD-1 antagonist antigen-binding molecule. In some embodiments, the at least one anti-ICM antigen-binding molecule comprises a PD-L1 antagonist antigen-binding molecule. In certain embodiments, the at least one anti-ICM antigen-binding molecule comprises a PD-1 antagonist antigen-binding molecule and a PD-L1 antagonist antigen-binding molecule. In some embodiments, the at least one anti-ICM antigen-binding molecule comprises a CTLA4 antagonist antigen-binding molecule. In other embodiments, the at least one anti-ICM antigen-binding molecule comprises a PD-1 antagonist antigen-binding molecule and a CTLA4 antagonist antigen-binding molecule. In other embodiments, the at least one anti-ICM antigen-binding molecule comprises a PD-L1 antagonist antigen-binding molecule and a CTLA4 antagonist. In specific embodiments, the anti-ICM antigen-binding molecule antagonizes an ICM that a Treg cell lacks expression of or expresses at a low level. In some of the same and other embodiments, the anti-ICM antigen-binding molecule antagonizes an ICM (e.g., PD-1 or PD-L1) that is expressed at a lower level on a Treg than CTLA4. In some of the same and other embodiments, the anti-ICM antigen-binding molecule antagonizes an ICM (e.g., PD-1 or PD-L1) that is expressed at a higher level on an immune effector cell (e.g., an effector T cell, macrophage, dendritic cell, B cell, etc.) than on a Treg. In representative examples of these embodiments, the at least one anti-ICM antigen-binding molecule antagonizes an ICM selected from one or both of PD-1 and PD-L1. Numerous anti-ICMs antigen-binding molecule are known in the art, any of which may be used in the practice of the present invention.

In specific embodiments, the anti-ICM antigen-binding molecule is selected from an anti-PD-1 antigen-binding molecule, an anti-PD-L1 antigen-binding molecule and an anti-CTLA4 antigen-binding molecule.

The anti-PD-1 antigen-binding molecule may be a MAb, non-limiting examples of which include nivolumab, pembrolizumab, pidilizumab, and MEDI-0680 (AMP-514), AMP-224, JS001-PD-1, SHR-1210, Gendor PD-1, PDR001, CT-011, REGN2810, BGB-317 or an antigen-binding fragment thereof. Alternatively, the anti-PD-1 antigen-binding molecule may be one that competes with nivolumab, pembrolizumab, pidilizumab, or MEDI-0680 for binding to PD-1.

In some embodiments, the anti-PD-1 antigen-binding molecule binds specifically to one or more amino acids of the amino acid sequence set forth in SEQ ID NO:9 (i.e., residues 62 to 86 of the native PD-1 sequence set forth in SEQ ID NO:10) and/or in the amino acid sequence set forth in SEQ ID NO:11 (i.e., residues 118 to 136 of the native PD-1 sequence set forth in SEQ ID NO:10). In some of the same embodiments and other embodiments, the anti-PD-1 antigen-binding molecule binds specifically to one or more amino acids of the amino acid sequence set forth in SEQ ID NO:12 (i.e., corresponding to residue 66 to 97 of the native PD-1 sequence set forth in SEQ ID NO:10).

In some embodiments, the anti-PD-L1 antigen-binding molecule is a MAb, non-limiting examples of which include durvalumab (MEDI4736), atezolizumab (Tecentriq), avelumab, BMS-936559/MDX-1105, MSB0010718C, LY3300054, CA-170, GNS-1480 and MPDL3280A, or an antigen-binding fragment thereof. In illustrative examples of this type, the anti-PD-L1 antigen-binding molecule binds specifically to one or more amino acids in the amino acid sequence set forth in SEQ ID NO:13 (i.e., residues 279 to 290 of the full length native PD-L1 amino acid sequence set forth in SEQ ID NO:14). Alternatively, the anti-PD-L1 antigen-binding molecule may be one that competes with any one of durvalumab (MEDI4736), atezolizumab (Tecentriq), avelumab, BMS-936559/MDX-1105, MSB0010718C, LY3300054, CA-170, GNS-1480 and MPDL3280A for binding to PD-L1.

In some embodiments, the anti-CTLA4 antigen-binding molecule is a MAb, representative examples of which include ipilimumab and tremelimumab, or an antigen-binding fragment thereof. Alternatively, the anti-CTLA4 antigen-binding molecule may be one that competes with ipilimumab or tremelimumab for binding to CTLA4. In illustrative examples of this type, the anti-CTLA4 antigen-binding molecule binds specifically to one or more amino acids in an amino acid sequence selected from the sequences set forth in any one of SEQ ID NO:15 (i.e., residues 25 to 42 of the full-length native CTLA4 amino acid sequence set forth in SEQ ID NO:16), SEQ ID NO:17 (i.e., residues 43 to 65 of the native CTLA4 sequence set forth in SEQ ID NO:16), and SEQ ID NO:18 (i.e., residues 96 to 109 of the native CTLA4 sequence set forth in SEQ ID NO:16).

In some embodiments, the therapeutic combination comprises, consists or consists essentially of a RANK antagonist antigen-binding molecule described herein and an anti-PD-1 antigen-binding molecule. In other embodiments, the therapeutic combination comprises, consists or consists essentially of a RANK antagonist antigen-binding molecule described herein and an anti-PD-L1 antigen-binding molecule. In still other embodiments, the therapeutic combination comprises, consists or consists essentially of a RANK antagonist antigen-binding molecule described herein, an anti-PD-1 antigen-binding molecule and an anti-PD-L1 antigen-binding molecule. In still other embodiments, the therapeutic combination comprises, consists or consists essentially of a RANK antagonist antigen-binding molecule described herein, an anti-PD-1 antigen-binding molecule and an anti-CTLA4 antigen-binding molecule. In other embodiments, the therapeutic combination comprises, consists or consists essentially of a RANK antagonist antigen-binding molecule described herein and an anti-PD-L1 antigen-binding molecule.

In some embodiments in which the RANK or ICM antigen-binding molecule is linked to an immunoglobulin constant chain (e.g., an IgG1, IgG2a, IgG2b, IgG3, or IgG4 constant chain). The immunoglobulin constant chain may comprise a light chain selected from a κ light chain or λ light chain; and a heavy chain selected from a γ1 heavy chain, γ2 heavy chain, γ3 heavy chain, and γ4 heavy chain.

In certain embodiments, the therapeutic combination comprises, consists or consists essentially of a RANK antagonist antigen-binding molecule described herein and two or more different anti-ICM antigen-binding molecules. In representative examples of this type, the therapeutic combination comprises, consists or consists essentially of a RANK antagonist antigen-binding molecule described herein and at least two of an anti-CTLA4 antigen-binding molecule, an anti-PD-1 antigen-binding molecule and an anti-PD-L1 antigen-binding molecule.

Components of the therapeutic combination may be in the form of discrete components. Alternatively, they may be fused or otherwise conjugated (either directly or indirectly) to one another.

In specific embodiments, the therapeutic combination is in the form of a multispecific antagonist agent, comprising the RANK antagonist antigen-binding molecule described herein and the at least one anti-ICM antigen-binding molecule. The multispecific agent may be a complex of two or more polypeptides. Alternatively, the multispecific agent may be a single chain polypeptide. The RANK antagonist antigen-binding molecule may be conjugated to the N-terminus or to the C-terminus of an individual anti-ICM antigen-binding molecule. The RANK antagonist antigen-binding molecule and anti-ICM antigen-binding molecule may be connected directly or by an intervening linker (e.g., a polypeptide linker). In advantageous embodiments, the multispecific antagonist agent comprises at least two antigen-binding molecules. Suitably, individual components of the multispecific antigen-binding molecules are in the form of recombinant molecules, including chimeric, humanized and human antigen-binding molecules.

In a related aspect, the present invention provides multispecific antigen-binding molecules for co-antagonizing RANK and at least one ICM. These multispecific antigen-binding molecules generally comprise, consist or consist essentially of a RANK antagonist antigen-binding molecule described herein and at least one anti-ICM antigen-binding molecule. The RANK antagonist antigen-binding molecule is suitably an antibody or antigen-binding fragment thereof that binds specifically to and antagonizes RANK. Individual anti-ICM antigen-binding molecules are suitably selected from antibodies or antigen-binding fragments that binds specifically to and antagonize a corresponding ICM. The antibody and/or antigen-binding fragments may be connected directly or by an intervening linker (e.g., a chemical linker or a polypeptide linker). An individual multispecific antigen-binding molecule may be in the form of a single chain polypeptide in which the antibodies or antigen-binding fragments are operably connected. Alternatively, it may comprise a plurality of discrete polypeptide chains that are linked to or otherwise associated with one another to form a complex. In some of the same and other embodiments, the multispecific antigen-binding molecules are bivalent, trivalent, or tetravalent.

Antigen-binding fragments that are contemplated for use in multispecific antigen-binding molecules may be selected from Fab, Fab′, F(ab′)2, and Fv molecules and complementarity determining regions (CDRs). In some embodiments, individual antibodies or antigen-binding fragments thereof comprise a constant domain that is independently selected from the group consisting of IgG, IgM, IgD, IgA, and IgE. Non-limiting examples of multispecific antigen-binding molecules suitably comprise a tandem scFv (taFv or scFv₂), diabody, dAb₂/VHH₂, knobs-in-holes derivative, Seedcod-IgG, heteroFc-scFv, Fab-scFv, scFv-Jun/Fos, Fab′-Jun/Fos, tribody, DNL-F(ab)₃, scFv₃-C_{H}1/C_{L}, Fab-scFv₂, IgG-scFab, IgG-scFv, scFv-IgG, scFv₂-Fc, F(ab′)₂-scFv₂, scDB-Fc, scDb-C_{H}3, db-Fc, scFv₂-H/L, DVD-Ig, tandAb, scFv-dhlx-scFv, dAb₂-IgG, dAb-IgG, dAb-Fc-dAb, tandab, DART, BIKE, TriKE, mFc-V_{H}, crosslinked MAbs, Cross MAbs, MAb₂, FIT-Ig, electrostatically matched antibodies, symmetric IgG-like antibodies, LUZ-Y, Fab-exchanged antibodies, or a combination thereof.

Suitable antigen-binding fragments may be linked to an immunoglobulin constant chain (e.g., IgG1, IgG2a, IgG2b, IgG3, and IgG4). In representative examples of this type, the immunoglobulin constant chain may comprise a light chain selected from a κ light chain and λ light chain, and/or a heavy chain selected from a γ1 heavy chain, γ2 heavy chain, γ3 heavy chain, and γ4 heavy chain.

In some embodiments in which the multispecific antigen-binding molecule antagonizes PD-1, the anti-PD-1 antibody or antigen-binding fragment thereof binds specifically to one or more amino acids of an amino acid sequence selected from SEQ ID NO:9 (i.e., residues 62 to 86 of the native human PD-1 sequence set forth in SEQ ID NO:10), SEQ ID NO:11 (i.e., residues 118 to 136 of the native human PD-1 sequence set forth in SEQ ID NO:10) and SEQ ID NO:12 (i.e., corresponding to residue 66 to 97 of the native human PD-1 sequence set forth in SEQ ID NO:10).

In some of the same and other embodiments, the anti-PD-1 antibody or antigen-binding fragment thereof comprises a heavy chain and a light chain of a MAb selected from nivolumab, pembrolizumab, pidilizumab, and MEDI-0680 (AMP-514), AMP-224, 3S001-PD-1, SHR-1210, Gendor PD-1, PDR001, CT-011, REGN2810, BGB-317 or antigen-binding fragments thereof.

In some embodiments in which the multispecific antigen-binding molecule antagonizes PD-L1, the anti-PD-L1 antibody or antigen-binding fragment thereof binds specifically to one or more amino acids of the amino acid sequence set forth in SEQ ID NO:13 (i.e., residues 279 to 290 of the native human PD-L1 amino acid sequence as set forth in SEQ ID NO:14). Illustrative antibodies and antigen-binding fragments of this type include those that comprise a heavy chain and a light chain of a MAb selected from durvalumab (MEDI4736), atezolizumab (Tecentriq), avelumab, BMS-936559/MDX-1105, MSB0010718C, LY3300054, CA-170, GNS-1480 and MPDL3280A, or antigen-binding fragments thereof.

In some embodiments in which the multispecific antigen-binding molecule antagonizes CTLA4, the anti-CTLA4 antibody or antigen-binding fragment thereof binds specifically to one or more amino acids of an amino acid sequence selected from SEQ ID NO:15 (i.e., residues 25 to 42 of the full-length native PD-CTLA4 amino acid sequence set forth in SEQ ID NO:16), SEQ ID NO:17 (i.e., residues 43 to 65 of the native CTLA4 sequence set forth in SEQ ID NO:16), and SEQ ID NO:18 (i.e., residues 96 to 109 of the native CTLA4 sequence set forth in SEQ ID NO:16). Illustrative antibodies and antigen-binding fragments of this type include those that comprise a heavy chain and a light chain of a MAb selected from ipilimumab and tremelimumab, or antigen-binding fragments thereof.

In some embodiments, the multispecific antigen-binding molecule comprises, consists or consists essentially of a RANK antagonist antigen-binding molecule described herein and an anti-PD-1 antigen-binding molecule. In other embodiments, the multispecific antigen-binding molecule comprises, consists or consists essentially of a RANK antagonist antigen-binding molecule described herein and an anti-PD-L1 antigen-binding molecule. In still other embodiments, the multispecific antigen-binding molecule comprises, consists or consists essentially of a RANK antagonist antigen-binding molecule described herein, an anti-PD-1 antigen-binding molecule and an anti-PD-L1 antigen-binding molecule. In still other embodiments, the multispecific antigen-binding molecule comprises, consists or consists essentially of a RANK antagonist antigen-binding molecule described herein, an anti-PD-1 antigen-binding molecule and an anti-CTLA4 antigen-binding molecule. In other embodiments, the multispecific antigen-binding molecule comprises, consists or consists essentially of a RANK antagonist antigen-binding molecule described herein and an anti-PD-L1 antigen-binding molecule.

In another aspect, the present invention provides methods of producing a therapeutic combination as broadly described above and elsewhere herein. These methods generally comprise combining a RANK antagonist antigen-binding molecule described herein and at least one anti-ICM antigen-binding molecule to thereby produce the therapeutic combination. In some embodiments, the methods comprise generating an antigen-binding molecule that binds specifically to and antagonizes a target polypeptide (e.g., RANK or an ICM) of the therapeutic combination (e.g., by immunizing an animal with an immunizing polypeptide comprising an amino acid sequence corresponding to an the target polypeptide; and identifying and/or isolating a B cell from the animal, which binds specifically to the target polypeptide or at least one region thereof; and producing the antigen-binding molecule expressed by that B cell). In non-limiting examples, the methods further comprise derivatizing the antigen-binding molecule so generated to produce a derivative antigen-binding molecule with the same epitope-binding specificity as the antigen-binding molecule. The derivative antigen-binding molecule may be selected from antibody fragments, illustrative examples of which include Fab, Fab′, F(ab′)₂, Fv, single chain (scFv), one-arm and domain antibodies (including, for example, shark and camelid antibodies), and fusion proteins comprising an antibody, and any other modified configuration of an immunoglobulin molecule that comprises an antigen binding/recognition site.

In some embodiments, the therapeutic combination or multispecific antigen-binding molecule is contained in a delivery vehicle (e.g., a liposome, a nanoparticle, a microparticle, a dendrimer or a cyclodextrin).

In still another aspect, the present invention provides constructs that comprise nucleic acid sequence encoding a multispecific antigen-binding molecule as described herein in operable connection with one or more control sequences. Suitable constructs are preferably in the form of an expression construct, representative examples of which include plasmids, cosmids, phages, and viruses.

Still another aspect of the invention provides host cells that contain constructs comprising a nucleic acid sequence encoding a multispecific antigen-binding molecule as described herein in operable connection with one or more control sequences.

In another aspect, the present invention provides pharmaceutical compositions comprising the therapeutic combination or multispecific antigen-binding molecule as broadly described above, and a pharmaceutically acceptable carrier. In some embodiments, the compositions further comprise at least one ancillary agent selected from a chemotherapeutic agent (e.g., selected from antiproliferative/antineoplastic drugs, cytostatic agents, agents that inhibit cancer cell invasion, inhibitors of growth factor function, anti-angiogenic agents, vascular damaging agents, etc.), or an immunotherapeutic agent (e.g., cytokines, cytokine-expressing cells, antibodies, etc.).

Still another aspect of the present invention provides methods for stimulating or augmenting immunity in a subject. These methods generally comprise, consist or consist essentially of administering to the subject an effective amount of the therapeutic combination or multispecific antigen-binding molecule as described herein, to thereby stimulate or augment immunity in the subject. In embodiments in which the RANK antagonist antigen-binding molecule and the at least one anti-ICM antigen-binding molecule of the therapeutic combination are provided as discrete components, the components are suitably administered concurrently to the subject. In illustrative examples of this type, the RANK antagonist antigen-binding molecule is administered simultaneously with the at least one anti-ICM antigen-binding molecule. In other illustrative examples, the RANK antagonist antigen-binding molecule and the at least one anti-ICM antigen-binding molecule are administered sequentially. For instance, the RANK antagonist antigen-binding molecule may be administered prior to administration of the at least one anti-ICM antigen-binding molecule. Suitably, the RANK antagonist antigen-binding molecule is administered after administration of the at least one anti-ICM antigen-binding molecule.

Typically, the stimulated or augmented immunity comprises a beneficial host immune response, illustrative examples of which include any one or more of the following: reduction in tumor size; reduction in tumor burden; stabilization of disease; production of antibodies against an endogenous or exogenous antigen; induction of the immune system; induction of one or more components of the immune system; cell-mediated immunity and the molecules involved in its production; humoral immunity and the molecules involved in its production; antibody-dependent cellular cytotoxicity (ADCC) immunity and the molecules involved in its production; complement-mediated cytotoxicity (CDC) immunity and the molecules involved in its production; natural killer cells; cytokines and chemokines and the molecules and cells involved in their production; antibody-dependent cytotoxicity; complement-dependent cytotoxicity; natural killer cell activity; and antigen-enhanced cytotoxicity. In representative examples of this type, the stimulated or augmented immunity includes a pro-inflammatory immune response.

Yet another aspect of the present invention provides methods for inhibiting the development or progression of immunosuppression or tolerance to a tumor in a subject. These methods generally comprise, consist or consist essentially of contacting the tumor with the therapeutic combination or multispecific antigen-binding molecule described herein, to thereby inhibit the development or progression of immunosuppression or tolerance to the tumor in the subject. Suitably, the therapeutic combination or multispecific antigen-binding molecule also contacts an antigen-presenting cell (e.g., a dendritic cell) that presents a tumor antigen to the immune system.

A further aspect of the present invention provides methods for inhibiting the development, progression or recurrence of a cancer in a subject. These methods generally comprise, consist or consist essentially of administering to the subject an effective amount of a therapeutic combination or multispecific antigen-binding molecule described herein, to thereby inhibit the development, progression or recurrence the cancer in the subject.

In a related aspect, the present invention provides methods for treating a cancer in a subject. These methods generally comprise, consist or consist essentially of administering to the subject an effective amount of a therapeutic combination or multispecific antigen-binding molecule described herein, to thereby treat the cancer.

Non-limiting examples of cancers that may be treated in accordance with the present invention include melanoma, breast cancer, colon cancer, ovarian cancer, endometrial and uterine carcinoma, gastric or stomach cancer, pancreatic cancer, prostate cancer, salivary gland cancer, lung cancer, hepatocellular cancer, glioblastoma, cervical cancer, liver cancer, bladder cancer, hepatoma, rectal cancer, colorectal cancer, kidney cancer, vulval cancer, thyroid cancer, hepatic carcinoma, anal carcinoma, penile carcinoma, testicular cancer, esophageal cancer, tumors of the biliary tract, head and neck cancer, and squamous cell carcinoma. In some particular embodiments, the cancer is a metastatic cancer.

In any of the above aspects involving administration of the therapeutic combination or multispecific antigen-binding molecule to a subject, the subject has suitably reduced or impaired responsiveness to immunomodulatory agents, for example a subject that has reduced or impaired responsiveness to ICM molecule antagonists (e.g., an anti-PD-1 or anti-PD-L1 immunotherapy).

In some of the methods of the invention, an effective amount of an ancillary anti-cancer agent is concurrently administered to the subject. Some suitable ancillary anti-cancer agents include a chemotherapeutic agent, external beam radiation, a targeted radioisotope, and a signal transduction inhibitor. However, any other known anti-cancer agent is equally as applicable for use with the methods of the present invention.

In a further aspect, the present invention provides kits for stimulating or augmenting immunity, for inhibiting the development or progression of immunosuppression or tolerance to a tumor, or for treating a cancer in a subject. These kits comprise any one or more of the therapeutic combinations, pharmaceutical compositions, and multispecific antigen-binding molecules as broadly described above and elsewhere herein.

### BRIEF DESCRIPTION OF THE DRAWINGS

FIG. 1 is a graphical representation showing an ELISA for reactivity of anti-RANK phagemid clones to human RANK-Fc [RANK AA sequence 30-212] or mouse RANK-Fc [RANK AA sequence 31-214]. Single point analyses were performed using 100 μL/well of phage solution per well in the ELISA (ELISA method as per Panousis et al., 2016, infra). Y-axis is O.D. A450 nm. Phagemid-Fab clones were tested for target binding by ELISA. Purified RANK-Fc or irrelevant human IgG antibody were coated at 2 pg/mL in MTPBS; pH 7.3 onto 96-well MaxiSorp™ ELISA (Thermo Fischer Scientific 439454) plates overnight at 4° C. Plates were blocked for 2 h at 37° C. with 200 μL/well of 5% skim milk/PBST, washed twice with PBST before incubation with phage supernatant (100 μL/well) for 90 min at room temperature. The phage supernatant was diluted 1:2 with skim milk/PBST. Plates were washed×5 with PBST prior to incubation with anti-M13-HRP antibody (Sino biological/Jomar 11973-MM05-100) diluted 1:10,000 in PBST. Plates were washed ×6 with PBST and signal developed with 100 pL TMB/E substrate (Merck Millipore ES001-500ML). The reaction was stopped with 2 M phosphoric acid (50 μL well) and measured at 450 nm.

FIG. 2 is a graphical representation showing inhibition of anti-RANK phagemid R03A03 binding to human RANK-Fc by RANKL. Recombinant soluble human RANKL was added to final well concentrations of 1 pM. The competition phage ELISA was performed as described previously except prior to addition of 50 μL/well of phage supernatant, an equal volume of competitor RANKL protein at 2 μM was added per well in 4% skim milk/PBST. Values were averages of duplicate determinations.

FIG. 3 is a graphical representation showing the inhibitory effects of anti-RANK 3A3 antibody on human RANKL-induced in vitro osteoclastogenesis. Murine BM cells cultured in the presence or absence of RANK-Fc as a positive control, IgG2a isotype control, non-blocking anti-RANK 3610 mAb or blocking, anti-RANK 3A3 antibody at concentrations from 1000 ng/mL to 7.8 ng/mL. Culture of BM cells was performed in DMEM supplemented with CSF-1 and human RANKL. Seven days later, TRAP+ multinucleated (more than three nuclei) cells were counted. Data were analyzed as means+SEM of triplicate cultures.

FIG. 4 is a graphical representation showing inhibitory effects of anti-RANK 3A3 antibody on mouse RANKL-induced in vitro osteoclastogenesis. Murine BM cells cultured in the presence or absence of anti-muRANKL IK22-5 mAb as a positive control, IgG2a isotype control, non-blocking anti-RANK 3610 mAb or blocking, anti-RANK 3A3 antibody at concentrations from 1000 ng/mL to 7.8 ng/mL. Culture of BM cells was performed in DMEM supplemented with CSF-1 and mouse RANKL. Seven days later, TRAP+ multinucleated (more than three nuclei) cells were counted. Data were analyzed as means+SEM of triplicate cultures.

FIG. 5 is a graphical representation showing that the combination of anti-PD-L1 and anti-RANK (3A3) mAbs restrain subcutaneous growth of tumors. Groups of C57BL/6 WT mice were injected subcutaneously with MCA1956 (1×10⁶ cells) on day 0. Mice were then treated i.p. on days 10, 14, 18 and 22 with either cIg (2A3 200 pg i.p.); anti-PD-L1 alone (10F9G2 rat IgG2b, 50 μg i.p.); anti-RANK alone (3A3 mouse IgG1D265A, 200 μg i.p.) or their combinations as indicated. Tumor growth was measured using a digital caliper, and tumor sizes are presented as mean+SEM for 5-6 mice per group.

FIG. 6 is a graphical representation showing that the combination of anti-PD-L1 and anti-RANK (3A3) mAbs restrain subcutaneous growth of tumors. Groups of C57BL/6 WT mice were injected subcutaneously with MC38-ovadim (1×10⁶ cells) on day 0. Mice were then treated i.p. on days 12, 16, 20 and 24 with either cIg (2A3 200 μg i.p.); anti-PD-L1 alone (10F9G2 rat IgG2b, 50 μg i.p.); anti-RANK alone (3A3 mouse IgG1D265A, 200 μg i.p.) or their combinations as indicated. Tumor growth was measured using a digital caliper, and tumor sizes are presented as mean+SEM for 5 mice per group.

Some figures and text contain color representations or entities. Color illustrations are available from the Applicant upon request or from an appropriate Patent Office. A fee may be imposed if obtained from a Patent Office.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

As used herein, “and/or” refers to and encompasses any and all possible combinations of one or more of the associated listed items, as well as the lack of combinations when interpreted in the alternative (or).

The term “antagonist” is used in the broadest sense, and includes any molecule that partially or fully blocks, inhibits, stops, diminishes, reduces, impedes, impairs or neutralizes one or more biological activities or functions of RANK or an ICM such as but not limited to binding, signaling, formation of a complex, proliferation, migration, invasion, survival or viability, in any setting including, in vitro, in situ, or in vivo. Likewise, the terms “antagonize”, “antagonizing” and the like are used interchangeably herein to refer to blocking, inhibiting stopping, diminishing, reducing, impeding, impairing or neutralizing an activity or function as described for example above and elsewhere herein. By way of example, “antagonize” can refer to a decrease of about 10%, 20%, 30%, 40%, 50%, 60%, 70%, 80%, 90% or 100% in an activity, or function.

The term “antibody”, as used herein, means any antigen-binding molecule or molecular complex comprising at least one complementarity determining region (CDR) that binds specifically to or interacts with a particular antigen (e.g., RANK or ICM). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H1}, C_{H2} and C_{H3}. Each light chain comprises a light chain variable region (which may be abbreviated as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of an antibody of the invention (or antigen-binding portion thereof) may be identical to the human germline sequences, or may be naturally or artificially modified. An amino acid consensus sequence may be defined based on a side-by-side analysis of two or more CDRs.

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

The terms “antigen-binding fragment”, “antigen-binding portion”, “antigen-binding domain” and “antigen-binding site” are used interchangeably herein to refer to a part of an antigen-binding molecule that participates in antigen-binding. These terms include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-C_{H3}, V_{H}-C_{H2}-C_{H3}; (Vii) V_{H}-C_{L}; (Viii) V_{L}-C_{H1}; (ix) V_{L}-C_{H2}, (X) V_{L}-C_{H3}; (xi) V_{L}-C_{H1}-C_{H2}; (XII) V_{L}-C_{H1}-C_{H2}-C_{H3}; (Xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format, including the exemplary bispecific antigen-binding molecule formats disclosed herein, may be adapted for use in the context of an antigen-binding fragment of an antibody of the present invention using routine techniques available in the art.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and their antigen-binding fragments. The term “antigen-binding molecule” includes antibodies and antigen-binding fragments of antibodies.

The term “bispecific antigen-binding molecule” refers to a multi-specific antigen-binding molecule having the capacity to bind to two distinct epitopes on the same antigen or on two different antigens. A bispecific antigen-binding molecule may be bivalent, trivalent, or tetravalent. As used herein, “valent”, “valence”, “valencies”, or other grammatical variations thereof, mean the number of antigen-binding sites in an antigen-binding molecule. These antigen recognition sites may recognize the same epitope or different epitopes. Bivalent and bispecific molecules are described in, e.g., Kostelny et al. J Immunol 148 (1992):1547, Pack and Plückthun Biochemistry 31 (1992) 1579, Gruber et al. J Immunol (1994) 5368, Zhu et al. Protein Sci 6 (1997):781, Hu et al. Cancer Res. 56 (1996):3055, Adams et al. Cancer Res. 53 (1993):4026, and McCartney, et al. Protein Eng. 8 (1995):301. Trivalent bispecific antigen-binding molecules and tetravalent bispecific antigen-binding molecules are also known in the art. See, e.g., Kontermann R E (ed.), Springer Heidelberg Dordrecht London New York, pp. 199-216 (2011). A bispecific antigen-binding molecule may also have valencies higher than 4 and are also within the scope of the present invention. Such antigen-binding molecules may be generated by, for example, dock and lock conjugation method. (Chang, C.-H. et al. In: Bispecific Antibodies. Kontermann R E (2011), supra).

By contrast, the term “monovalent antigen-binding molecule” refers to an antigen-binding molecule that binds to a single epitope of an antigen. Monovalent antigen-binding molecule are typically incapable of antigen-crosslinking.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site. An antigen-binding site of an antigen-binding molecule described herein typically binds specifically to an antigen and more particularly to an epitope of the antigen.

The phrase “binds specifically” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule bind to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules such as immunoglobulins such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976)).

The term “chimeric”, when used in reference to a molecule, means that the molecule contains portions that are derived from, obtained or isolated from, or based upon two or more different origins or sources. Thus, a polypeptide is chimeric when it comprises two or more amino acid sequences of different origin and includes (1) polypeptide sequences that are not found together in nature (i.e., at least one of the amino acid sequences is heterologous with respect to at least one of its other amino acid sequences), or (2) amino acid sequences that are not naturally adjoined.

“Cluster of Differentiation 38” (CD38) (also known as cyclic ADP ribose hydrolase, ADPRC1 and ADPRC 1) is a glycoprotein found on the surface of many immune cells (white blood cells), including CD4⁺, CD8⁺, B lymphocytes, myeloid and natural killer cells. CD38 also functions in cell adhesion, signal transduction and calcium signaling. CD38 is a multifunctional ectoenzyme that catalyzes the synthesis and hydrolysis of cyclic ADP-ribose (cADPR) from NAD⁺ to ADP-ribose in addition to synthesis of NAADP from NADP+. The term “CD38” includes fragments of CD38, as well as related polypeptides, which include, but are not limited to, allelic variants, splice variants, derivative variants, substitution variants, deletion variants, and/or insertion variants, fusion polypeptides, and interspecies homologs. In certain embodiments, a CD38 polypeptide includes terminal residues, such as, but not limited to, leader sequence residues, targeting residues, amino terminal methionine residues, lysine residues, tag residues and/or fusion protein residues. In preferred embodiments, “CD38” as used herein includes human CD38 (hCD38), variants, isoforms, and species homologs of hCD38, and analogs having at least one common epitope with hCD38. The complete hCD38 sequence can be found under UniProt Accession No. P28907.

“Cluster of Differentiation 103” (CD103) (also known as integrin, alpha E (ITGAE), HUMINAE, integrin subunit alpha E) is an integrin protein that in human is encoded by the ITGAE gene. CD103 binds integrin beta 7 (β7-ITGB7) to form the complete heterodimeric integrin molecule aE07. The term “CD103” includes fragments of CD103, as well as related polypeptides, which include, but are not limited to, allelic variants, splice variants, derivative variants, substitution variants, deletion variants, and/or insertion variants, fusion polypeptides, and interspecies homologs. In certain embodiments, a CD103 polypeptide includes terminal residues, such as, but not limited to, leader sequence residues, targeting residues, amino terminal methionine residues, lysine residues, tag residues and/or fusion protein residues. In preferred embodiments, “CD103” as used herein includes human CD103 (hCD103), variants, isoforms, and species homologs of hCD103, and analogs having at least one common epitope with hCD103. The complete hCD103 sequence can be found under UniProt Accession No. P3850.

“Cluster of Differentiation 163” (CD163) (also known as M130, MM130, SCARI1) is the high affinity scavenger receptor for the hemoglobin-haptoglobin complex and in the absence of haptoglobin—with lower affinity—for hemoglobin alone. It also is a marker of cells from the monocyte/macrophage lineage and, in particular, is a marker of M2-like immunosuppressive myeloid cells. CD163 functions as innate immune sensor for gram-positive and gram-negative bacteria. The term “CD163” includes fragments of CD163, as well as related polypeptides, which include, but are not limited to, allelic variants, splice variants, derivative variants, substitution variants, deletion variants, and/or insertion variants, fusion polypeptides, and interspecies homologs. In certain embodiments, a CD163 polypeptide includes terminal residues, such as, but not limited to, leader sequence residues, targeting residues, amino terminal methionine residues, lysine residues, tag residues and/or fusion protein residues. In preferred embodiments, “CD163” as used herein includes human CD163 (hCD163), variants, isoforms, and species homologs of hCD163, and analogs having at least one common epitope with hCD163. The complete hCD163 sequence can be found under UniProt Accession No. Q86VB7.

“Cluster of Differentiation 200” (CD200) (also known OX-2 membrane glycoprotein, MOX1, MOX2, MRC, OX-2) is a human protein encoded by the CD200 gene. The protein encoded by this gene is a type-1 membrane glycoprotein, which contains two immunoglobulin domains, and thus belongs to the immunoglobulin superfamily. This gene regulates myeloid cell activity and delivers an inhibitory signal for the macrophage lineage in diverse tissues. The term “CD200” includes fragments of CD200, as well as related polypeptides, which include, but are not limited to, allelic variants, splice variants, derivative variants, substitution variants, deletion variants, and/or insertion variants, fusion polypeptides, and interspecies homologs. In certain embodiments, a CD200 polypeptide includes terminal residues, such as, but not limited to, leader sequence residues, targeting residues, amino terminal methionine residues, lysine residues, tag residues and/or fusion protein residues. In preferred embodiments, “CD200” as used herein includes human CD200 (hCD200), variants, isoforms, and species homologs of hCD200, and analogs having at least one common epitope with hCD200. The complete hCD200 sequence can be found under UniProt Accession No. P41217.

“Cluster of Differentiation 206” (CD206) (also known as mannose receptor), is a C-type lectin primarily present on the surface of myeloid cells including macrophages and immature dendritic cells. The receptor recognizes terminal mannose, N-acetylglucosamine and fucose residues on glycans attached to proteins found on the surface of some microorganisms, playing a role in both the innate and adaptive immune systems. Additional functions include clearance of glycoproteins from the circulation, including sulfated glycoprotein hormones and glycoproteins released in response to pathological events. The mannose receptor recycles continuously between the plasma membrane and endosomal compartments in a clathrin-dependent manner. The term “CD206” includes fragments of CD206, as well as related polypeptides, which include, but are not limited to, allelic variants, splice variants, derivative variants, substitution variants, deletion variants, and/or insertion variants, fusion polypeptides, and interspecies homologs. In certain embodiments, a CD206 polypeptide includes terminal residues, such as, but not limited to, leader sequence residues, targeting residues, amino terminal methionine residues, lysine residues, tag residues and/or fusion protein residues. In preferred embodiments, “CD206” as used herein includes human CD206 (hCD206), variants, isoforms, and species homologs of hCD206, and analogs having at least one common epitope with hCD206. The complete hCD206 sequence can be found under UniProt Accession No. P22897.

By “coding sequence” is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

As used herein, the term “complementarity determining regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined for example by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). The term “polypeptide complex” or “protein complex,” as used herein, refers to a trimer, tetramer, pentamer, hexamer, heptamer, octamer, nonamer, decamer, undecamer, dodecamer, or higher order oligomer.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. In some embodiments, the phrase “consisting essentially of” in the context of a recited subunit sequence (e.g., amino acid sequence) indicates that the sequence may comprise at least one additional upstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids) and/or at least one additional downstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids), wherein the number of upstream subunits and the number of downstream subunits are independently selectable.

As used herein, the terms “conjugated”, “linked”, “fused” or “fusion” and their grammatical equivalents, in the context of joining together of two more elements or components or domains by whatever means including chemical conjugation or recombinant means (e.g., by genetic fusion) are used interchangeably. Methods of chemical conjugation (e.g., using heterobifunctional crosslinking agents) are known in the art.

The term “constant domains” or “constant region” as used within the current application denotes the sum of the domains of an antibody other than the variable region. The constant region is not directly involved in binding of an antigen, but exhibits various immune effector functions.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element” or “control sequence” is meant nucleic acid sequences (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

By “corresponds to” or “corresponding to” is meant a nucleic acid sequence that displays substantial sequence identity to a reference nucleic acid sequence (e.g., at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g., at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

“Cytotoxic T-lymphocyte-associated protein 4 (CTLA4)” (also known as ALPSS, CD, CD152, CELIAC3, CTLA-4, GRD4, GSE, IDDM12), refers to a protein receptor that, functioning as an immune checkpoint, downregulates immune responses. CTLA4 is constitutively expressed in T regulatory cells (Tregs) but only upregulated in conventional T cells after activation. It acts as an “off” switch when bound to CD80 or CD86 on the surface of antigen-presenting cells. The term “CTLA4” as used herein includes human CTLA4 (hCTLA4), variants, isoforms, and species homologs of hCTLA4, and analogs having at least one common epitope with hCTLA4. The complete hCTLA4 sequence can be found under UniProt Accession No. P16410.

The term “DART” (dual affinity retargeting reagent) refers to an immunoglobulin molecule that comprises at least two polypeptide chains that associate (especially through a covalent interaction) to form at least two epitope-binding sites, which may recognize the same or different epitopes. Each of the polypeptide chains of a DART comprise an immunoglobulin light chain variable region and an immunoglobulin heavy chain variable region, but these regions do not interact to form an epitope binding site. Rather, the immunoglobulin heavy chain variable region of one (e.g., the first) of the DART polypeptide chains interacts with the immunoglobulin light chain variable region of a different (e.g., the second) DART polypeptide chain to form an epitope binding site. Similarly, the immunoglobulin light chain variable region of one (e.g., the first) of the DART polypeptide chains interacts with the immunoglobulin heavy chain variable region of a different (e.g., the second) DART polypeptide chain to form an epitope binding site. DARTs may be monospecific, bispecific, trispecific, etc., thus being able to simultaneously bind one, two, three or more different epitopes (which may be of the same or of different antigens). DARTs may additionally be monovalent, bivalent, trivalent, tetravalent, pentavalent, hexavalent, etc., thus being able to simultaneously bind one, two, three, four, five, six or more molecules. These two attributes of DARTs (i.e., degree of specificity and valency may be combined, for example to produce bispecific antibodies (i.e., capable of binding two epitopes) that are tetravalent (i.e., capable of binding four sets of epitopes), etc. DART molecules are disclosed in more detail in International PCT Publication Nos. WO 2006/113665, WO 2008/157379, and WO 2010/080538.

By “effective amount,” in the context of treating or preventing a disease or condition (e.g., a cancer) is meant the administration of an amount of active agent to a subject, either in a single dose or as part of a series or slow release system, which is effective for the treatment or prevention of that disease or condition. The effective amount will vary depending upon the health and physical condition of the subject and the taxonomic group of individual to be treated, the formulation of the composition, the assessment of the medical situation, and other relevant factors.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include a RNA product resulting from transcription of a DNA molecule, a protein resulting from translation of a RNA molecule, a protein resulting from transcription of a DNA molecule to form a RNA product and the subsequent translation of the RNA product, or a protein resulting from transcription of a DNA molecule to provide a RNA product, processing of the RNA product to provide a processed RNA product (e.g., mRNA) and the subsequent translation of the processed RNA product.

The terms “epitope” and “antigenic determinant” are used interchangeably herein to refer to a region of an antigen that is bound by an antigen-binding molecule or antigen-binding fragment thereof. Epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance (see, e.g., Morris G. E., Epitope Mapping Protocols, Meth Mol Biol, 66 (1996)). A preferred method for epitope mapping is surface plasmon resonance. Bispecific antibodies may be bivalent, trivalent, or tetravalent. When used herein in the context of bispecific antibodies, the terms “valent”, “valence”, “valencies”, or other grammatical variations thereof, mean the number of antigen binding sites in an antibody molecule. These antigen recognition sites may recognize the same epitope or different epitopes. Bivalent and bispecific molecules are described in, for example, Kostelny et al., (1992) J Immunol 148:1547; Pack and Plückthun (1992) Biochemistry 31:1579; Hollinger et al., 1993, supra, Gruber et al., (1994) J Immunol 5368, Zhu et al., (1997) Protein Sci 6:781; Hu et al., (1996) Cancer Res 56:3055; Adams et al., (1993) Cancer Res 53:4026; and McCartney et al., (1995) Protein Eng 8:301. Trivalent bispecific antibodies and tetravalent bispecific antibodies are also known in the art (see, e.g., Kontermann R E (ed.), Springer Heidelberg Dordrecht London New York, 199-216 (2011)). A bispecific antibody may also have valencies higher than 4 and are also within the scope of the present invention. Such antibodies may be generated by, for example, dock and lock conjugation method (see, Chang, C.-H. et al. In: Bispecific Antibodies. Kontermann R E (ed.), Springer Heidelberg Dordrecht London New York, pp. 199-216 (2011)).

As used herein, the terms “function,” “functional” and the like refer to a ligand-binding, multimerizing, activating, signaling, biologic, pathologic or therapeutic function.

“Framework regions” (FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

“Galectin-9” (Gal9) (also referred tro as LGALS9, HUAT, LGALS9A) is a tandem-repeat type galectin with two carbohydrate-recognition domains, which modulates a variety of biological functions including cell aggregation and adhesion, as well as apoptosis of tumor cells. Galectin-9 also has an anti-proliferative effect on cancer cells and interacts with T cell immunoglobulin mucin-3 (Tim-3) to negatively regulate T cell responses by promoting CD8⁺ T cell exhaustion and inducing expansion of myeloid-derived suppressor cells. These mechanisms are involved in tumor growth and escape from immunity. In many solid cancers, the loss of galectin-9 expression is closely associated with metastatic progression, and treatment with recombinant galectin-9 prevents metastatic spread in various preclinical cancer models. The term “Gal9” includes fragments of Gal9, as well as related polypeptides, which include, but are not limited to, allelic variants, splice variants, derivative variants, substitution variants, deletion variants, and/or insertion variants, fusion polypeptides, and interspecies homologs. In certain embodiments, a Gal9 polypeptide includes terminal residues, such as, but not limited to, leader sequence residues, targeting residues, amino terminal methionine residues, lysine residues, tag residues and/or fusion protein residues. In preferred embodiments, “Gal9” as used herein includes human Gal9 (hGal9), variants, isoforms, and species homologs of hGal9, and analogs having at least one common epitope with hGal9. The complete hGal9 sequence can be found under UniProt Accession No. O00182.

“Herpesvirus entry mediator” (HVEM) (also known as tumor necrosis factor receptor superfamily member 14 (TNFRSF14), ATAR, CD270, HVEA, HVEM, LIGHTR, TR2, tumor necrosis factor receptor superfamily member 14, TNF receptor superfamily member 14) is a human cell surface receptor of the TNF-receptor superfamily. This receptor was identified as a cellular mediator of herpes simplex virus (HSV) entry. Binding of HSV viral envelope glycoprotein D (gD) to this receptor protein has been shown to be part of the viral entry mechanism. The cytoplasmic region of this receptor was found to bind to several TRAF family members, which may mediate the signal transduction pathways that activate the immune response. The term “HVEM” includes fragments of HVEM, as well as related polypeptides, which include, but are not limited to, allelic variants, splice variants, derivative variants, substitution variants, deletion variants, and/or insertion variants, fusion polypeptides, and interspecies homologs. In certain embodiments, a HVEM polypeptide includes terminal residues, such as, but not limited to, leader sequence residues, targeting residues, amino terminal methionine residues, lysine residues, tag residues and/or fusion protein residues. In preferred embodiments, “HVEM” as used herein includes human HVEM (hHVEM), variants, isoforms, and species homologs of hHVEM, and analogs having at least one common epitope with hHVEM. The complete hHVEM sequence can be found under UniProt Accession No. Q92956.

As used herein, the term “higher” in reference to a measurement of a cellular marker, or biomarker, refers to a statistically significant and measurable difference in the level of a biomarker measurement compared with a reference level where the biomarker measurement is greater than the reference level. The difference is suitably at least about 10%, or at least about 20%, or of at least about 30%, or of at least about 40%, or at least about 50%, or at least about 60%, or at least about 70%, or at least about 80%, or at least about 90%.

The term “immune checkpoint molecule” includes both receptors and ligands that function as an immune checkpoint. Immune checkpoints are the immune escape mechanism to prevent the immune system from attacking its own body. Immune checkpoint receptors are present on T cells, and interact with immune checkpoint ligands expressed on antigen-presenting cells. T cells recognize an antigen presented on the MHC molecule and are activated to generate an immune reaction, whereas an interaction between immune checkpoint receptor and ligand that occurs in parallel with the above controls the activation of T cells. Exemplary immune checkpoint molecule include, without limitation, PD-1, PD-L1, PD-L2, CTLA-4, A2AR, A2BR, B7-H3 CD276, VTCN1, BTLA, IDO, KIR, LAG3, TIM-3, VISTA, CD73, CD96, CD155, DNAM-1, CD112, CRTAM, TNFRS4 (OX40, CD134), TNFSF4 (OX40L), CD244, CD160, GITR, GITRL, ICOS, GAL-9, 4-1BBL (CD137L), 4-1BB (CD137), CD70, CD27L, CD28, B7-1 (CD80), B7-2 (CD86), SIRP-1, IAP (CD47), BLAST-1 (CD48), CD244; CD40, CD40L, HVEM, TMIGD2, HHLA2, VEGI, TNFRS25, ICOLG (B7RP1) and TIGIT. In specific embodiments, the immune checkpoint molecule is PD-1, PD-L1 or CTLA-4.

The term “immune effector cells” in the context of the present invention relates to cells which exert effector functions during an immune reaction. For example, such cells secrete cytokines and/or chemokines, kill microbes, secrete antibodies, recognize infected or cancerous cells, and optionally eliminate such cells. For example, immune effector cells comprise T cells (cytotoxic T cells, helper T cells, tumor infiltrating T cells), B-cells, natural killer (NK) cells, lymphokine-activated killer (LAK) cells, neutrophils, macrophages, and dendritic cells.

The term “immune effector functions” in the context of the present invention includes any functions mediated by components of the immune system that result, for example, in the killing of virally infected cells or tumor cells, or in the inhibition of tumor growth and/or inhibition of tumor development, including inhibition of tumor dissemination and metastasis. Preferably, the immune effector functions in the context of the present invention are T-cell mediated effector functions. Such functions comprise in the case of a helper T-cell (CD4⁺ T-cell) the recognition of an antigen or an antigen peptide derived from an antigen in the context of MHC class II molecules by T-cell receptors, the release of cytokines and/or the activation of CD8⁺ lymphocytes (CTLs) and/or B-cells, and in the case of CTL the recognition of an antigen or an antigen peptide derived from an antigen in the context of MHC class I molecules by T-cell receptors, the elimination of cells presented in the context of MHC class I molecules, i.e., cells characterized by presentation of an antigen with class I MHC, for example, via apoptosis or perforin-mediated cell lysis, production of cytokines such as IFN-γ and TNF-α, and specific cytolytic killing of antigen expressing target cells.

The term “immune system” refers to cells, molecular components and mechanisms, including antigen-specific and non-specific categories of the adaptive and innate immune systems, respectively, that provide a defense against damage and insults and matter, the latter comprised of antigenic molecules, including but not limited to tumors, pathogens, and self-reactive cells. By “adaptive immune system” refers to antigen-specific cells, molecular components and mechanisms that emerge over several days, and react with and remove a specific antigen. The adaptive immune system develops throughout a host's lifetime. The adaptive immune system is based on leukocytes, and is divided into two major sections: the humoral immune system, which acts mainly via immunoglobulins produced by B cells, and the cell-mediated immune system, which functions mainly via T cells.

By “linker” is meant a molecule or group of molecules (such as a monomer or polymer) that connects two molecules and often serves to place the two molecules in a desirable configuration. In specific embodiments, a “peptide linker” refers to an amino acid sequence that connects two proteins, polypeptides, peptides, domains, regions, or motifs and may provide a spacer function compatible with the spacing of antigen-binding fragments so that they can bind specifically to their cognate epitopes). In certain embodiments, a linker is comprised of about two to about 35 amino acids, for instance, or about four to about 20 amino acids or about eight to about 15 amino acids or about 15 to about 25 amino acids.

As used herein, the term “lower” in reference to a measurement of a cellular marker, or biomarker, refers to a statistically significant and measurable difference in the level of a biomarker measurement compared with a reference level where the biomarker measurement is less than the reference level. The difference is suitably at least about 10%, or at least about 20%, or of at least about 30%, or of at least about 40%, or at least about 50%, or at least about 60%, or at least about 70%, or at least about 80%, or at least about 90%.

“Negative”, “positive” and “low” expression levels as they apply to markers are defined as follows. Cells with negative expression (i.e., “−”) or that “lack expression” are defined herein as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one,” or “FMO,” staining. Cells with expression greater than the 95th percentile of expression observed with an isotype control antibody using the FMO staining procedure described above are herein defined as “positive” (i.e., “+”). There are various populations of cells broadly defined as “positive.” For example, cells with low expression (i.e., “low” or “lo”) are generally defined as those cells with observed expression above the 95th percentile determined using FMO staining with an isotype control antibody and within one standard deviation of the 95th percentile of expression observed with an isotype control antibody using the FMO staining procedure described above. The term “low” or “10” in relation to an ICM (e.g., PD-1, PD-L1, etc.) refers to a cell or population of cells (e.g., Treg cells, including T cells in the tumor microenvironment) that expresses the ICM at a lower level than one or more other distinct cells or populations of cells (e.g., immune effector cells such as T-cells, 6-cells, natural killer (NK) cells, NK T (NKT) cells, monocytes, macrophages, and dendritic cells (DCs); as well as tumor cells). For example, it is known that in the tumor microenvironment CTLA4 is expressed at a significantly higher level on Treg than PD-1 and PD-1 is expressed at a significantly higher level on immune effector cells, including effector T cells, than on Treg (Jacobs et al., 2009. Neuro-Oncology 11(4): 394-402).

“Macrophage receptor with collagenous structure” (MARCO) (also known as SCARA2 and SR-A6) is a class A scavenger receptor that is found on particular subsets of macrophages. Scavenger receptors are pattern recognition receptors (PRRs) and are most commonly found on immune cells. Their defining feature is that they bind to polyanions and modified forms of a type of cholesterol called low-density lipoprotein (LDL). MARCO is able to bind and phagocytose these ligands and pathogen-associated molecular patterns (PAMPs), leading to the clearance of pathogens as well as causing downstream effects in the cell that lead to inflammation. As part of the innate immune system, MARCO clears, or scavenges, pathogens and leads to inflammatory responses. The scavenger receptor cysteine-rich (SRCR) domain at the end of the extracellular side of MARCO is responsible for ligand binding and the subsequent immune responses. MARCO expression on macrophages is also associated with diseases since Alzheimer's disease is associated with decreased response within the cell when a ligand binds to MARCO. The term “MARCO” includes fragments of MARCO, as well as related polypeptides, which include, but are not limited to, allelic variants, splice variants, derivative variants, substitution variants, deletion variants, and/or insertion variants, fusion polypeptides, and interspecies homologs. In certain embodiments, a MARCO polypeptide includes terminal residues, such as, but not limited to, leader sequence residues, targeting residues, amino terminal methionine residues, lysine residues, tag residues and/or fusion protein residues. In preferred embodiments, “MARCO” as used herein includes human MARCO (hMARCO), variants, isoforms, and species homologs of hMARCO, and analogs having at least one common epitope with hMARCO. The complete hMARCO sequence can be found under UniProt Accession No. Q9UEW3.

As used herein, the term “microenvironment” refers to the connective, supportive framework of a biological cell, tissue, or organ. As used herein, the term “tumor microenvironment” or “TME” refers to any and all elements of the tumor milieu that creates a structural and or functional environment for the malignant process to survive and/or expand and/or spread. Generally, the term “tumor microenvironment” or “TME” refers to the cellular environment in which the tumor exists, including the area immediately surrounding fibroblasts, leukocytes and endothelial cells and the extracellular matrix (ECM). Accordingly, cells of a tumor microenvironment comprise malignant cells in association with non-malignant cells that support their growth and survival. The non-malignant cells, also called stromal cells, occupy or accumulate in the same cellular space as malignant cells, or the cellular space adjacent or proximal to malignant cells, which modulate tumor cell growth or survival. The term “stromal cells” include fibroblasts, leukocytes and vascular cells. Non-malignant cells of the tumor microenvironment include fibroblasts, epithelial cells, vascular cells (including blood and lymphatic vascular endothelial cells and pericytes), resident and/or recruited inflammatory and immune (e.g., macrophages, dendritic cells, granulocytes, lymphocytes, etc.). These cells and especially activated fibroblasts actively participate in metastasis development.

The term “monoclonal antibody” (Mab), as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), and as modified by the somatic hybridization method as set forth above; or may be made by other recombinant DNA methods (such as those described in U.S. Pat. No. 4,816,567).

The term “multispecific antigen-binding molecule” is used in its broadest sense and specifically covers an antigen-binding molecule with specificity for at least two (e.g., 2, 3, 4, etc.) different epitopes (i.e., is capable of specifically binding to two, or more, different epitopes on one antigen or is capable of specifically binding to epitopes on two, or more, different antigens).

The term “myeloid cell” as used herein refers to cells of the myeloid lineage or derived therefrom. The myeloid lineage includes a number of morphologically, phenotypically, and functionally distinct cell types including different subsets of granulocytes (neutrophils, eosinophils, and basophils), monocytes, macrophages, erythrocytes, megakaryocytes, and mast cells. In certain embodiments, the myeloid cell is a cell derived from a cell line of myeloid lineage.

As used herein, the term “myopathy” refers to a muscular disease in which the muscle fibers do not function properly, typically resulting in muscular weakness. Myopathies include muscular diseases that are neuromuscular or musculoskeletal in nature. In some embodiments, the myopathy is an inherited myopathy. Inherited myopathies include, without limitation, dystrophies, myotonias, congenital myopathies (e.g., nemaline myopathy, multi/minicore myopathy, and centronuclear myopathy), mitochondrial myopathies, familial periodic myopathies, inflammatory myopathies and metabolic myopathies (e.g., glycogen storage diseases and lipid storage disorder). In some embodiments, the myopathy is an acquired myopathy. Acquired myopathies include, without limitation, external substance induced myopathy (e.g., drug-induced myopathy and glucocorticoid myopathy, alcoholic myopathy, and myopathy due to other toxic agents), myositis (e.g., dermatomyositis, polymyositis and inclusion body myositis), myositis ossificans, rhabdomyolysis, and myoglobinurias, and disuse atrophy. In some embodiments, the myopathy is disuse atrophy, which may be caused by bone fracture (e.g., a hip fracture) or by nerve injury (e.g., spinal cord injury (SCI)). In some embodiments the myopathy is related to a disease or disorder such as amyotrophic lateral sclerosis (ALS), spinal muscular atrophy (SMA), cachexia syndromes due to renal failure, AIDS, cardiac conditions and/or cancer. In some embodiments the myopathy is related to ageing.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence. Likewise, “operably connecting” a first antigen-binding fragment to a second antigen-binding fragment encompasses positioning and/or orientation of the antigen-binding fragments in such a way as to permit binding of each antigen-binding fragment to its cognate epitope.

The term “osteopenic disorder” refers to conditions with decreased calcification and/or bone density, and is used to refer to all skeletal systems in which the condition is noted. Representative osteopenic disorders include osteoporosis, osteopenia, Paget's disease, lytic bone metastases, periodontitis, rheumatoid arthritis, and bone loss due to immobilization. In addition to these bone disorders, certain cancers are known to increase osteoclast activity and induce bone resorption, such as breast, prostate, and multiple myeloma. These cancers are now known to produce factors that result in the over-expression of RANKL in the bone, and lead to increased osteoclast numbers and activity.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

“Programmed Death-1 (PD-1)” (also known as CD279, PD1, SLEB2, hPD-1, hPD-I, and hSLE1) refers to an immuno-inhibitory receptor belonging to the CD28 family. PD-1 is expressed predominantly on previously activated T cells in vivo, and binds to two ligands, PD-L1 and PD-L2. The term “PD-1” includes fragments of PD-1, as well as related polypeptides, which include, but are not limited to, allelic variants, splice variants, derivative variants, substitution variants, deletion variants, and/or insertion variants, fusion polypeptides, and interspecies homologs. In certain embodiments, a PD-1 polypeptide includes terminal residues, such as, but not limited to, leader sequence residues, targeting residues, amino terminal methionine residues, lysine residues, tag residues and/or fusion protein residues. In preferred embodiments, “PD-1” includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” (also known as CD274, B7-H, B7H1, PDCD1L1, PDCD1LG1, PDL1 and CD274 molecule) is one of two cell surface glycoprotein ligands for PD-1 (the other being PD-L2) that downregulate T cell activation and cytokine secretion upon binding to PD-1. The term “PD-L1” includes fragments of PD-L1, as well as related polypeptides, which include, but are not limited to, allelic variants, splice variants, derivative variants, substitution variants, deletion variants, and/or insertion variants, fusion polypeptides, and interspecies homologs. In certain embodiments, a PD-1 polypeptide includes terminal residues, such as, but not limited to, leader sequence residues, targeting residues, amino terminal methionine residues, lysine residues, tag residues and/or fusion protein residues. In preferred embodiments, “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

The terms “polypeptide,” “proteinaceous molecule”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

“Receptor activator of NF-κB ligand (RANKL)” (also known as tumor necrosis factor ligand superfamily member 11 (TNFSF11), TNF-related activation-induced cytokine (TRANCE), osteoprotegerin ligand (OPGL) and osteoclast differentiation factor (ODF)) refers to a polypeptide that inter alia promotes formation of osteoclasts through binding to receptor activator of NF-κB (RANK). The term “RANKL” includes fragments of RANKL, as well as related polypeptides, which include, but are not limited to, allelic variants, splice variants, derivative variants, substitution variants, deletion variants, and/or insertion variants, fusion polypeptides, and interspecies homologs. In certain embodiments, a RANKL polypeptide includes terminal residues, such as, but not limited to, leader sequence residues, targeting residues, amino terminal methionine residues, lysine residues, tag residues and/or fusion protein residues. The term RANKL includes human RANKL (hRANKL), variants, isoforms, and species homologs of hRANKL, and analogs having at least one common epitope with hRANKL. The complete hRANKL sequence can be found under UniProt Accession No. O14788.

“Receptor activator of NF-κB (RANK)” (also known as tumor necrosis factor receptor superfamily, member 11a, NF-κB activator, CD265, FEO, LOH18CR1, ODFR, OFE, OPTB7, OSTS, PDB2, and TRANCER) refers to a polypeptide that is a receptor for RANK-Ligand (RANKL) and part of the RANK/RANKL/osteoprotegerin (OPG) signaling pathway that regulates osteoclast differentiation and activation. It is associated with bone remodeling and repair, immune cell function, lymph node development, thermal regulation, and mammary gland development. The term “RANK” includes fragments of RANK, as well as related polypeptides, which include, but are not limited to, allelic variants, splice variants, derivative variants, substitution variants, deletion variants, and/or insertion variants, fusion polypeptides, and interspecies homologs. In certain embodiments, a RANK polypeptide includes terminal residues, such as, but not limited to, leader sequence residues, targeting residues, amino terminal methionine residues, lysine residues, tag residues and/or fusion protein residues. The term RANK includes human RANK (hRANK, variants, isoforms, and species homologs of hRANK, and analogs having at least one common epitope with hRANK. The complete hRANK sequence can be found under UniProt Accession No. Q9Y6Q6.

As used herein, “recombinant” antigen-binding molecule means any antigen-binding molecule whose production involves expression of a non-native DNA sequence encoding the desired antibody structure in an organism, non-limiting examples of which include tandem scFv (taFv or scFv2), diabody, dAb2/VHH2, knob-into-holes derivatives, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDB-C_{H3}, db-Fc, scFv₂-H/L, DVD-Ig, tandAb, scFv-dhlx-scFv, dAb₂-IgG, dAb-IgG, dAb-Fc-dAb, CrossMabs, MAb₂, FIT-Ig, and combinations thereof.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. In some aspects, the Treg is a CD4⁺CD25⁺Foxp3⁺ T cell.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting an immune response to a cancer. However, it will be understood that the aforementioned terms do not imply that symptoms are present.

The terms “therapeutic combination”, “in combination” and the like, with reference to the agents of the present invention (e.g., RANK antagonist antigen-binding molecule, anti-ICM antigen-binding molecule, anti-AMA antigen-binding molecule, etc.) include any combination, including combinations in which the agents are physically connected (e.g., covalently connected in a single polypeptide or non-covalent connected in a complex), or are present as discrete components in a single composition or are in different compositions to be administered simultaneously, together or separately, or separately at different times, as part of a regimen. Typically, each such agent in the therapeutic combinations of the present invention will be present in a pharmaceutical composition comprising a pharmaceutically acceptable carrier. The agents in a therapeutic combination of the present invention are provided in dosage forms such that the beneficial effect of each agent is realized by a subject at the desired time.

By “treatment,” “treat,” “treated” and the like is meant to include both prophylactic and therapeutic treatment, including but not limited to preventing, relieving, altering, reversing, affecting, inhibiting the development or progression of, ameliorating, or curing (1) a disease or condition associated with the presence or aberrant expression of a target antigen, or (2) a symptom of the disease or condition, or (3) a predisposition toward the disease or condition, including conferring protective immunity to a subject.

As used herein, the term “trispecific antibody” refers to an antibody that comprises at least a first antigen-binding domain with specificity for a first epitope, a second antigen-binding domain with specificity for a second epitope, and a third antigen-binding domain with specificity for a third epitope e.g., RANK and any two of CTLA4, PD-1, and PD-L1. The first, second, and third epitopes are not the same (i.e., are different targets (e.g., proteins)), but can all be present (e.g., co-expressed) on a single cell or on at least two cells.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In an exemplary embodiment, the cancer is selected from prostate, lung, pancreatic, breast, ovarian and bone cancer.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Each embodiment described herein is to be applied mutatis mutandis to each and every embodiment unless specifically stated otherwise.

### 2. Abbreviations

The following abbreviations are used throughout the application:

| | |
|---|---|
| aa= | amino acid(s) |
| CDR= | complementarity determining regions |
| CD38= | cluster of differentiation 38 |
| CD103= | cluster of differentiation 103 |
| CD163= | cluster of differentiation 163 |
| CD200= | cluster of differentiation 200 |
| CD206= | cluster of differentiation 206 |
| CTLA4= | cytotoxic T-lymphocyte-associated protein 4 |
| DC= | dendritic cell |
| Fc= | constant region |
| FR= | framework |
| Gal9= | galectin-9 |
| h= | hour |
| HVEM= | herpesvirus entry mediator |
| ICM= | immune checkpoint molecule |
| Ig= | immunoglobulin |
| MAb= | monoclonal antibody |
| MARCO= | macrophage receptor with collagenous structure |
| OPG= | osteoprotegerin |
| PD-1= | programmed death 1 |
| PD-L1= | programmed death ligand 1 |
| RANK= | receptor activator of NF-κB |
| RANKL= | receptor activator of NF-κB ligand |
| s= | seconds |
| TME= | tumor microenvironment |
| V_{H}= | heavy chain variable domain |
| V_{L}= | light chain variable domain |

### 3. RANK Antagonist Antigen-Binding Molecules

The present invention discloses antigen-binding molecules that bind to and antagonize RANK function, including antagonizing the RANKL/RANK signaling pathway. These antagonist antigen-binding molecules can be used alone, or in combination with other agents, in a range of applications including in the treatment or prophylaxis of osteopenic disorders, myopathies and cancers.

In specific embodiments, the antigen-binding molecules disclosed herein comprise:
(1) a heavy chain variable region (VH) comprising a VHCDR1 amino acid sequence of GFTFSSYAMH [SEQ ID NO:3], a VHCDR2 amino acid sequence of VVSYDGSTKS [SEQ ID NO:4], and a VHCDR3 amino acid sequence of DPALRYFDWGYFQH [SEQ ID NO:5], and a light chain variable region (VL) comprising a VLCDR1 amino acid sequence of SGDKLGDKYVC [SEQ ID NO:6], a VLCDR2 amino acid sequence of GDSERPS [SEQ ID NO:7], and a VLCDR3 amino acid sequence of QAWDSTTPL [SEQ ID NO:8];
(2) a VH that comprises the amino acid sequence EVQLVESGGGVVQPGTSLRLSCAASGFTFSSYAMHWVRQAPGKGLEWVAVVSYDGSTKSYADS MKGRFTISRDNSKNTLYLQMNSLRAEDTAVYYCAKDPALRYFDWGYFQHWGQGTLVTVSS [SEQ ID NO:1], and a VL that comprises the amino acid sequence SYELTQPPSVSVSPGQTASITCSGDKLGDKYVCWYQQKPGQSPVLVIYGDSERPSGIPERFSGSN SGNTATLTISGTRAVDEADYYCQAWDSTTPLFGGGTNLTVL [SEQ ID NO:2];
(3) a VH with at least 90% (including at least 91% to 99% and all integer percentages therebetween) sequence identity to the amino acid sequence of SEQ ID NO:1, and a VL with at least 90% (including at least 91% to 99% and all integer percentages therebetween) sequence identity to the amino acid sequence of SEQ ID NO:2;
(4) a VH with at least 90% (including at least 91% to 99% and all integer percentages therebetween) sequence identity to the amino acid sequence of a framework region other than each CDR in the amino acid sequence of SEQ ID NO:1, and a VL with at least 90% (including at least 91% to 99% and all integer percentages therebetween) sequence identity to the amino acid sequence of a framework region other than each CDR in the amino acid sequence of SEQ ID NO:2; or
(5) a VH that comprises an amino acid sequence comprising a deletion, substitution or addition of one or more (e.g., 1, 2, 3, 4 or 5) amino acids in the sequence of a framework region other than at each CDR in the amino acid sequence of SEQ ID NO:1, and a VL that comprises an amino acid sequence comprising a deletion, substitution or addition of one or more (e.g., 1, 2, 3, 4 or 5) amino acids in the sequence of a framework region other than at each CDR in the amino acid sequence of SEQ ID NO:2.

TNFR superfamily (TNFRSF) members, of which RANK is a member, are generally activated by binding to their respective ligands that oligomerize TNFRSF, leading to activation. This structural interplay between ligand and receptor is challenging for therapeutic antibodies because the bivalent nature of antibodies can dimerize and agonize rather than antagonize their intended target. Indeed, oligomerization of TNFR superfamily (TNFRSF), for which RANK is one member, can lead to agonistic activity (Wajant, H., 2015, Cell Death Differ. 22(11):1727-1741) and this includes examples of antibody-mediated oligomerization of RANK, leading to agonistic activation (Chypre, 2016, supra). Thus, in some embodiments, the antigen-binding molecules are monovalent and are unable to cross-link or multimerize RANK. Monovalent antigen-binding molecules have the capacity to bind only one antigen molecule, thus avoiding or reducing the risk of receptor-crosslinking and activation. As the term is used herein, a monovalent antigen-binding molecule can also comprise more than one antigen binding site, e.g., two antigen binding sites, but the binding sites must be for different antigens, such that the antigen-binding molecule can only bind one molecule of RANK at a time. The antigen-binding domain of a monovalent antigen-binding molecule can comprise a V_{H} and a V_{L} domain, but in some embodiments may comprise only a single immunoglobulin variable domain, i.e., a V_{H} or a V_{L} domain, that has the capacity to bind RANKL without the need for a corresponding V_{L} or V_{H} domain, respectively.

Non-limiting monovalent antigen-binding molecules include: a Fab fragment consisting of V_{L}, V_{H}, C_{L} and C_{H1} domains; a Fab′ fragment consisting of V_{L}, V_{H}, C_{L} and C_{H1} domains, as well as a portion of a C_{H2} domain; an Fd fragment consisting of V_{H} and C_{H1} domains; an Fv fragment consisting of V_{L} and V_{H} domains of a single arm of an antibody; a single-chain antibody molecule (e.g., scFab and scFv); a single domain antibody (dAb) fragment (Ward et al., 1989 Nature 341:544-546), which consists of a V_{H} domain; and a one-armed antibody, such as described in US20080063641 (Genentech) or other monovalent antibody, e.g., such as described in WO2007048037 (Amgen).

In specific embodiments, the antagonist antigen-binding molecule comprises an Fv fragment. The Fv fragment is the smallest unit of an immunoglobulin molecule with function in antigen-binding activities. An antigen-binding molecule in scFv (single chain fragment variable) format consists of variable regions of heavy (V_{H}) and light (V_{L}) chains, which are joined together by a flexible peptide linker that can be easily expressed in functional form in an expression host such as E. coli and mammalian cells, allowing protein engineering to improve the properties of scFv such as increase of affinity and alteration of specificity (Ahmed et al., 2012. Clin Dev Immunol. 2012:980250). In the scFv construction, the order of the domains can be either V_{H}-linker-V_{L} or V_{L}-linker-V_{H} and both orientations can applied.

Most linker sequences used in scFvs are multimers of the pentapeptide GGGGS (or G4S or Gly4Ser). Those include the 15-mer (G4S)3 (Huston et al., 1988. Proc Natl Acad Sci USA. 85(16), 5879-83), the 18-mer GGSSRSSSSGGGGSGGGG (Andris-Widhopf et al., “Generation of human scFv antibody libraries: PCR amplification and assembly of light- and heavy-chain coding sequences.” Cold Spring Harbor Protocols, 2011(9)) and the 20-mer (G4S)4 (Schaefer et al., “Construction of scFv Fragments from Hybridoma or Spleen Cells by PCR Assembly.” In: Antibody Engineering, R. Kontermann and S. Dubel, Springer Verlag, Heidelberg, Germany (2010) pp. 21-44). Many other sequences have been proposed, including sequences with added functionalities, e.g. an epitope tag or an encoding sequence containing a Cre-Lox recombination site or sequences improving scFv properties, often in the context of particular antibody sequences.

Cloning of the scFv is usually done by a two-step overlapping PCR (also known as Splicing by Overlap Extension or SOE-PCR), as described (Schaefer et al., 2010, supra). The VH and VL domains are first amplified and gel-purified and secondarily assembled in a single step of assembly PCR. The linker is generated either by overlap of the two inner primers or by adding a linker primer whose sequence covers the entire linker or more (three-fragment assembly PCR).

In some embodiments, the RANK antagonist scFv molecule comprises CDR sequences derived from the from the V_{H} and V_{L} sequences of the anti-RANK phagemid clone 3A3 described herein, as set out in Table 1.

TABLE 1

| Heavy chain | | Light chain | |
|---|---|---|---|
| CDR1 | GFTFSSYAMH | CDR1 | SGDKLGDKYVC |
| | [SEQ ID NO: 3] | | [SEQ ID NO: 6] |
| CDR2 | VVSYDGSTKS | CDR2 | GDSERPS |
| | [SEQ ID NO: 4] | | [SEQ ID NO: 7] |
| CDR3 | DPALRYFDWGYFQH | CDR3 | QAWDSTTPL |
| | [SEQ ID NO: 5] | | [SEQ ID NO: 8] |

In a representative example of this type, the RANK antagonist antigen-binding molecule comprises the sequence:

[SEQ ID NO: 17]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSSGGGGSGGGGSGGGGSsyeltqppsysy
spgqtasitcsgdklgdkyvcwyqqkpgqspvlviygdserpsgiperfs
gsnsgntatItisgtravdeadyycqawdsttplfgggtnltvl,

wherein:
Uppercase regular text corresponds to the variable heavy chain amino acid sequence of the anti-RANK MAb 3A3,
GGGGSGGGGSGGGGS [SEQ ID NO:18] is a flexible linker
Lowercase text corresponds to the variable light chain amino acid sequence of the anti-RANK MAb 3A3.

ScFvs may be recombinantly produced for example in E. coli or mammalian hosts upon cloning of the protein coding sequence for the scFv in the context of appropriate expression vectors with appropriate translational, transcriptional start sites and, in the case of mammalian expression, a signal peptide sequence.

In other embodiments, the RANK antagonist antigen-binding molecule consists or consists essentially of a single antigen-binding fragment (Fab) and a Fc region, wherein the Fc region comprises a first and a second Fc polypeptide, and wherein the first and second Fc polypeptides are present in a complex. This strategy has been successfully applied for anti-c-MET antibody, which demonstrated monovalent binding to c-MET and avoided c-MET agonism, as described for example by Merchant et al. (2013. Proc Natl Acad Sci USA. 110(32):E2987-96).

Recombinant expression of Fc-containing monovalent antigen-binding molecules can often lead to undesirable bivalent, homodimer contaminants. Strategies to inhibit formation of homodimers are known including methods that introduce mutations into immunoglobulin constant regions to create altered structures that support unfavorable interactions between polypeptide chains and suppress unwanted Fc homodimer formation. Non-limiting examples of this strategy to promote heterodimerization include the introduction of knobs-into-holes (KIH) structures into the two polypeptides and utilization of the naturally occurring heterodimerization of the C_{L} and C_{H1} domains (see, Kontermann, supra, pp. 1-28 (2011) Ridgway et al., 1996. Protein Eng. 9(7):617-21; Atwell et al., 1997. J Mol Biol. 270(1):26-35; as described in WO 2005/063816). These KIH mutations promote heterodimerization of the knob containing Fc and the hole containing heavy chain, improving the assembly of monovalent antibody and reducing the level of undesired bivalent antibody.

Modifications in the Fc domain of antagonistic anti-RANK human antibodies as described above would reduce Fc receptor binding and therefore reduce the potential for agonistic cross-linking of RANK. Different antibodies against CD40 protein, another TNFR superfamily (TNFRSF) member with high homology to RANK, have different functional antagonistic vs. agonistic properties and indicate that agonism of TNFRS can be conferred by anti-TNFR antibodies upon Fc-mediated crosslinking. For instance, the precise TNFR CRD epitope on CD40 in combination with isotype was shown to dictate anti-CD40 mAb activity such that CRD1 binding mAbs are agonistic as IgG2 or with FcgRIIB crosslinking (Yu et al., 2018, Cancer Cell 33:664-675). The so-called ‘LALA’ double mutation (Leu234Ala together with Leu235Ala) in human IgG (including IgG1) will significantly impair Fc receptor binding and effector function (Lund et al., 1991, J. Immunol. 147, 2657-2662; Lund et al., 1992, Mol. Immunol. 29:53-59). For human IgG4, engineering mutations S228P/L235E variant (SPLE) has previously demonstrated minimal FcγR binding (Newman et al., 2001, Clin. Immunol. 98, 164-174). Mutations in IgG1 or IgG4 Fc domains can be combined, for instance combining the LALA mutations in human IgG1 with a mutation at P329G or combining the SPLE mutation in human IgG4 with a mutation at P329G, will completely abolished FcγR and C1q interactions (Schlothauer et al., 2016, Protein Eng Des. Sel. 29, 457-466).

In some embodiment, the RANK antagonist is an anti-RANK antigen-binding molecule (e.g., a MAb or an antigen-binding fragment thereof), in which each of the IgG1 Fc chains of the antibody carries P329G, L235A, L234A (P329G LALA) mutations or each of the IgG4 Fc chains carries P329G, S228P, L235E mutations, in order to abolish any undesired cross-linking or immune effector function of the antibody, e.g., antibody-dependent cell-meditated cytotoxicity (ADCC), phagocytosis (ADCP) and complement dependent cytotoxicity (CDC).

Thus, in some embodiments, the present invention contemplates monovalent RANK antagonist antigen-binding molecules produced by co-expression of a light chain, heavy chain and a truncated Fc domain. Suitably, the heavy chain incorporates hole mutations and P329G LALA mutations, while the truncated Fc domain incorporates knob mutations and P329G LALA mutations. In some embodiments, the anti-RANK antibody comprises (a) a first polypeptide comprising the amino acid sequence of SEQ ID NO:1 (3A3 V_{H} sequence), a CH1 sequence and a first Fc polypeptide and (b) a second polypeptide comprising the amino acid sequence of SEQ ID NO:2 (3A3 V_{L} sequence), and a CL1 sequence. In some embodiments, the anti-RANK antibody further comprises (c) a third polypeptide comprising a second Fc polypeptide.

In vitro screens for agonistic activity of RANK antagonist antigen-binding molecules including an anti-RANK arm could be performed using bivalent or monovalent antibody forms of a RANK antagonist antigen-binding molecule in the RANK-Fas Jurkat assay, as described (Schneider et al., 2014, supra; Chypre et al., 2016, supra).

In one embodiment of constructing a monovalent RANK antagonist antigen-binding molecule, three constructs are made. First, the heavy chain (V_{H}) domains of 3A3 are directly fused in tandem with the truncated heavy chain (C_{H1}-C_{H2}-C_{H3}) of a human IgG1 molecule (e.g., atezolizumab) at the NH₂-terminus, in which the heavy chain C_{H3} domain is altered at position 407 (Y407A), termed the “hole” to promote KiH heterodimerization of the heavy chains. The second construct is V_{L} of 3A3 directly fused in tandem with CL of the human IgG1 molecule (e.g., atezolizumab) and the third construct is truncated heavy chain (C_{H2}-C_{H3}) of the human IgG1 molecule (e.g., atezolizumab) in which one of the heavy chain C_{H3} domain is altered at position 366 (T366W), termed the “knob” to promote KiH heterodimerization of the heavy chains. Both heavy chain constructs include L234A, L235A, P329G substitutions for reduced FcγR and C1q interactions.

In non-limiting examples:

The first construct consists of heavy chain (V_{H}) domains of 3A3 directly fused in tandem with the truncated heavy chain (C_{H1}-C_{H2}-C_{H3}) of atezolizumab, in which the heavy chain C_{H3} domain is altered at position 407 (Y407A), termed the “hole” to promote KiH heterodimerization of the heavy chains, has the following amino acid sequence:

[SEQ ID NO: 19]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSSastkgpsvfplapsskstsggtaalgc
lykdyfpepvtvswnsgaltsgvhtfpavlqssglyslssvvtypssslg
tqtyicnvnhkpsntkvdkkvepkscdkthtcppcpapeAAggpsvflfp
pkpkdtlnnisrtpevtcvvvdvshedpevkfnwyvdgvevhnaktkpre
eqyastyrvvsvltylhqdwlngkeykckvsnkalGapiektiskakgqp
repqvytlppsreemtknqvsltclykgfypsdiavewesngqpennykt
tppvldsdgsfflAskltvdksrwqqgnvfscsvmhealhnhytqkslsl
spgk,

wherein:
the mature amino acid sequence of the anti-RANK antibody (3A3) VH sequence is shown in capital letters,
the constant region (C_{H1}-C_{H2}-C_{H3}) of atezolizumab is shown in lowercase letters,
the L234A, L235A, P329G substitutions for reduced FcγR and C1q interactions and the Y407A “hole” substitution are in bold uppercase text.

The second construct is V_{L} of 3A3 directly fused in tandem with C_{L} of atezolizumab has the following amino acid sequence:

[SEQ ID NO: 20]
SYELTQPPSVSVSPGQTASITCSGDKLGDKYVCWYQQKPGQSPVLVIYGD
SERPSGIPERFSGSNSGNTATLTISGTRAVDEADYYCQAWDSTTPLFGGG
TNLTVLrtvaapsvfifppsdeqlksgtasyycllnnfypreakvqwkvd
nalqsgnsqesvteqdskdstyslsstltlskadyekhkvyacevthqgl
sspytksfnrgec,

wherein:
the mature amino acid sequence of the anti-RANK antibody (3A3) V_{L} sequence is shown in capital letters,
the constant region (C_{L}) of atezolizumab light chain is shown in lowercase letters.

The third construct is truncated heavy chain (C_{H2}-C_{H3}) of atezolizumab in which the heavy chain C_{H3} domain is altered at position 366 (T366W), termed the “knob” to promote KiH heterodimerization of the heavy chains has the following amino acid sequence:

[SEQ ID NO: 21]
CPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCKVSN
KALGAPIEKTISKAKGQPREPQVYTLPPSREEMTKNQVSLWCLVKGFYPS
DIAVEWESNGQPENNYKTTPPVLDSDGSFFLYSKLTVDKSRWQQGNVFSC
SVMHEALHNHYTQKSLSLSPGK,

wherein:
the mature amino acid sequence of the constant region (C_{H2}-C_{H3}) of atezolizumab is shown in capital letters,
the L234A, L235A, P329G substitutions for reduced FcγR and C1q interactions and the T366W “knob”” substitution are in bold uppercase text.

Expression of this monovalent molecule which binds and antagonizes RANK can be achieved for example in E. coli or mammalian hosts upon cloning of the protein coding sequences of the constructs in the context of appropriate expression vectors with appropriate translational, transcriptional start sites and, in the case of mammalian expression, a signal peptide sequence. Expression and purification of such constructs are described (Merchant et al., 2013, supra).

Another strategy that avoids cross-linking of a monovalent binding interaction includes the generation of Fc variants in the context of an Fc/scFv-Fc agent. Heterodimeric Fc-based monospecific antibodies (mAbs) with monovalent antigen binding have been generated by fusion of the scFv to the N-terminus of only one Fc chain (Fc/scFv-Fc) (Moore et al., 2011. MAbs. 3(6): 546-557; Ha et al., 2016. Front Immunol. 7: 394). In order to produce a heterodimeric, monovalent Fc/scFv-Fc agent, DNA constructs are designed encoding two different immunoglobulin polypeptides: (i) an Fc (Hinge-C_{H2}-C_{H3}-) and (ii) an scFv-Fc (V_{H}-linker-V_{L}-Hinge-C_{H2}-C_{H3′}). Here the two different C_{H3} domains, C_{H3′} and C_{H3″}, represent asymmetric changes to generate “Knobs-into-holes” structures, which facilitate heterodimerization of polypeptide chains by introducing large amino acids (knobs) into one chain of a desired heterodimer and small amino acids (holes) into the other chain of the desired heterodimer. Both constructs include L234A, L235A, P329G substitutions for reduced FcγR and C1q interactions.

In one embodiment of generating a monovalent, heterodimeric Fc/scFv-Fc anti-RANK antagonist, two constructs encoding two different immunoglobulin polypeptides are designed:

The first construct consists of the truncated heavy chain (Hinge-C_{H2}-C_{H3}) of a human IgG1 (e.g., atezolizumab), in which the heavy chain C_{H3} domain is altered at position 407 (Y407A), termed the “hole” to promote KiH heterodimerization of the heavy chains and includes the L234A, L235A, P329G substitutions, has the following amino acid sequence:

[SEQ ID NO: 22]
EPKSCDKTHTastkgpsvfplapsskstsggtaalgclvkdyfpepvtvs
wnsgaltsgvhtfpavlqssglyslssvvtvpssslgtqtyicnvnhkps
ntkvdkkvepkscdkthtcppcpapeAAggpsvflfppkpkdtlmisrtp
evtcvvvdvshedpevkfnwyvdgvevhnaktkpreeqyastyrvvsvlt
vlhqdwlngkeykckvsnkalGapiektiskakgqprepqvytlppsree
mtknqvsltclvkgfypsdiavewesngqpennykttppvldsdgsfflA
skltvdksrwqqgnvfscsvmhealhnhytqkslslspgk,

wherein:
the C_{H2}-C_{H3} sequence of atezolizumab is shown in lowercase letters,
the hinge region AA sequence of atezolizumab is shown in underlined, capital letters,
the L234A, L235A, P329G substitutions for reduced FcγR and C1q interactions and the Y407A “hole” substitution are in bold uppercase text.

The second construct consists of a scFv portion (V_{H}-linker-V_{L}) derived from the V_{H} and V_{L} sequences of anti-RANK 3A3 directly fused in tandem with the truncated heavy chain (Hinge-C_{H2}-C_{H3′}) sequences of a human IgG1 (e.g., atezolizumab), in which the heavy chain C_{H3} domain is altered at position 366 (T366W), termed the “knob” to promote KiH heterodimerization of the heavy chains and includes the L234A, L235A, P329G substitutions, has the following amino acid sequence:

[SEQ ID NO: 23]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSSGGGGSGGGGSGGGGSsyeltqppsvsv
spgqtasitcsgdklgdkyvcwyqqkpgqspvlviygdserpsgiperfs
gsnsgntatltisgtravdeadyycqawdsttplfgggtnltylEPKSCD
KTHTASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVV
VDVSHEDPEVKFNWYVDGVEVHNAKTKPREEQYASTYRVVSVLTVLHQDW
LNGKEYKCKVSNKALGAPIEKTISKAKGQPREPQVYTLPPSREEMTKNQV
SLWCLVKGFYPSDIAVEWESNGQPENNYKTTPPVLDSDGSFFLYSKLTVD
KSRWQQGNVFSCSVMHEALHNHYTQKSLSLSPGK,

wherein:
Uppercase regular text corresponds to the variable heavy chain amino acid sequence of the anti-RANK MAb 3A3,
GGGGSGGGGSGGGGS [SEQ ID NO:18] is a flexible linker
Lowercase text corresponds to the variable light chain amino acid sequence of the anti-RANK MAb 3A3,
the amino acid sequence of the hinge and constant region (C_{H2}-C_{H3}) of atezolizumab is shown in underlined capital letters,
the L234A, L235A, P329G substitutions for reduced FcγR and C1q interactions and the T366W “knob”” substitution are in bold uppercase text.

Expression and purification of a monovalent, heterodimeric Fc/scFv-Fc anti-RANK antagonist can be achieved by sub-cloning cDNAs encoding the above constructs into an appropriate mammalian expression vector, including appropriate signal peptide encoding sequences, and produced in mammalian cells, such as HEK-293 cells as described (Moore et al., 2011, MAbs 3, 546-557).

### 4. Therapeutic Combinations

The present inventors have disclosed in co-pending International Application No. PCT/AU2018/050557 filed 5 Jun. 2018 that co-antagonizing RANKL/RANK and an immune checkpoint molecule (ICM) results in a synergistic enhancement in the immune response to a cancer. Thus, the RANK antagonist antigen-binding molecules disclosed herein and anti-ICM antigen-binding molecules are contemplated for use in compositions for stimulating or augmenting an immune response to a cancer in a subject. The compositions generally employ (1) a RANK antagonist antigen-binding molecule disclosed herein, and (2) at least one anti-ICM antigen-binding molecule. The compositions take advantage of a newly identified synergy between the RANKL/RANK and ICM pathways, which results in an increased localization of CD8⁺ T-cells at the site of a tumor or cancer. Advantageously, the synergistic compositions suitably stimulate an enhancement of effector cell function, including for example, an enhanced effector T-cell function includes the production of Th1-type cytokines (e.g., IFN-γ and/or IL-2) and increased proportion of polyfunctional T-cells.

The present inventors have also shown in PCT/AU2018/050557 that the anti-tumor efficacy of anti-RANKL mAb IK22/5 was abrogated in mice lacking BatF3, suggesting an essential role for CD103⁺ DC-mediated cross-presentation. In addition, flow cytometry analysis of CD11c⁺/MHCII⁺ DC from tumors revealed that 100% of RANK-positive DC also expressed PDL-1 and CD103. A similar analysis indicated a significant enrichment for CD206 expression on RANK-positive tumor-infiltrating macrophages. These data are consistent with a mechanism of action whereby blocking RANK/RANKL disrupts an immunosuppressive or tolerogenic axis in the TME between RANK-expressing myeloid cells (e.g., DC or macrophages) and RANKL-expressing cells, such as tumor cells, lymphocytes, lymph node cells or other stromal components.

The tolerogenic nature of RANK signaling in myeloid cells in human cancers has been demonstrated by experimental observation. Human DCs cultured with RANKL-expressing cancer cell lines derived from genital tract squamous cell carcinoma (SCC) had a more immature and tolerogenic phenotype (Demoulin et al., 2015. Oncoimmunology 4, e1008334). These DCs were characterized by higher levels of immunoglobulin-like transcript 3 and the immunoregulatory cytokine IL-10 than those that were cultured with normal keratinocytes. The RANKL-RANK interaction was partially responsible for inducing this phenotype, as it was partially reversible through addition of the soluble RANKL decoy receptor OPG to the co-cultures. In human extramammary Paget disease (EMPD), an uncommon intraepithelial adenocarcinoma, RANK expression within the tumor is mostly co-localized with the macrophage markers CD163 (also known as scavenger receptor cysteine-rich type 1 protein M130), arginase-1 (Arg1), and CD206 (macrophage mannose receptor 1), suggesting that the RANK-expressing cells are immunosuppressive M2 type tumor-associated macrophages (TAMs) (Kambayashi et al., 2015. J. Invest. Dermatol. 135, 2547-2550). Accordingly, the present inventors further propose therapeutic combinations comprising (1) a RANK antagonist antigen-binding molecule described herein and (2) at least one antigen-binding molecule that binds specifically to an antigen that is co-expressed with RANK on the surface of myeloid cells (see, for example, An et al., 2016. Blood 128(12):1590-603; Fujimura et al., 2015. J. Invest. Dermatol. 135:2884-2887; Matsushita et al., 2010. Cancer Immunol Immunother 59:875; Georgoudaki et al., 2016. Cell Rep. 15(9):2000-2011) representative examples of which include PD-L1, CD206, CD103, CD200, Gal9, HVEM, CD38, CD163 and MARCO (also interchangeably referred to herein as “auxiliary myeloid antigens” (AMA)). Accordingly, the RANK antagonist antigen-binding molecules disclosed herein are also contemplated for use in combination with one or more anti-AMA antigen-binding molecules in compositions and methods for stimulating or augmenting immunity (e.g., to a cancer), for inhibiting the development or progression of immunosuppression or tolerance (e.g., to a tumor), or for inhibiting the development, progression or recurrence of cancer. These methods suitably comprise contacting a myeloid cell with a therapeutic combination comprising the RANK antagonist antigen-binding molecules disclosed herein in combination with one or more anti-AMA antigen-binding molecules.

The therapeutic combination may be in the form of a single composition (e.g., a mixture) comprising each of the RANK antagonist antigen-binding molecules and the at least one anti-ICM or AMA antigen-binding molecule. Alternatively, the RANK antagonist antigen-binding molecule and the at least one anti-ICM antigen-binding molecule may be provided as discrete components in separate compositions.

Suitable anti-ICM or AMA antigen-binding molecules may be selected from antibodies and their antigen-binding fragments, including recombinant antibodies, monoclonal antibodies (MAbs), chimeric antibodies, humanized antibodies, human antibodies, and antigen-binding fragments of such antibodies.

For application in humans, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g., a variable domain) derived from one species (e.g., mouse) fused to a domain (e.g., the constant domains) derived from a different species (e.g., human).

“Humanized antibodies” refer to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (see, Jones et al., Nature 321:522-525 (1986); Riechmann et al., Nature 332:323-329 (1988); and Presta, Curr Op Struct Biol 2:593-596 (1992)). Humanization can be essentially performed following the method of Winter et al. (see, Jones et al., supra; Riechmann et al., supra); and Verhoeyen et al., Science 239:1534-1536 (1988)), by substituting rodent CDRs or CDR sequences for the corresponding sequences of a human antibody. Furthermore, technologies have been developed for creating antibodies based on sequences derived from the human genome, for example by phage display or using transgenic animals (see, International Patent Publication No. WO 90/05144; Marks et al., (1991) By-passing immunization. Human antibodies from V-gene libraries displayed on phage, J Mol Biol, 222, 581-597; Knappik et al., J Mol Biol 296: 57-86, 2000; Carmen and Jermutus, Concepts in antibody phage display, Briefings in Functional Genomics and Proteomics 2002 1(2):189-203; Lonberg and Huszar, Human antibodies from transgenic mice. Int Rev Immunol 1995; 13(1):65-93; Bruggemann and Taussig, Production of human antibody repertoires in transgenic mice, Curr Opin Biotechnol 1997 8(4): 455-8). Such antibodies are “human antibodies” in the context of the present invention.

Any suitable anti-ICM antigen-binding molecule that can be used in therapy is contemplated for use in the practice of the present invention. The ICM that is antagonized by the therapeutic combinations of the present invention include any one or more of the inhibitory ICMs selected from: PD-1, PD-L1, PD-L2, CTLA-4, A2AR, A2BR, CD276, VTCN1, BTLA, IDO, KIR, LAG3, TIM-3, VISTA, CD73, CD96, CD155, DNAM-1, CD112, CRTAM, OX40, OX40L, CD244, CD160, GITR, GITRL, ICOS, GAL-9, 4-1BBL, 4-1BB, CD27L, CD28, CD80, CD86, SIRP-1, CD47, CD48, CD244, CD40, CD40L, HVEM, TMIGD2, HHLA2, VEGI, TNFRS25 and ICOLG. Suitably, in embodiments in which therapeutic combination comprises a RANKL antagonist and a single ICM antagonist, the ICM is other than CTLA-4.

In some preferred embodiments, an anti-ICM antigen-binding molecule included in the therapeutic combination is an anti-PD-1 antigen-binding molecule. In this regard, an “anti-PD-1 antigen-binding molecule” includes any antigen-binding molecule that blocks binding of PD-L1 (for example, PD-L1 expressed the surface of a cancer cell) to PD-1 that is expressed on an immune cell (for example, a T-cell, B-cell, or NKT cell). Alternative names or synonyms for PD-1 include PDCD1, PD1, CD279 and SLEB2. A representative mature amino acid sequence of human PD-1 (UniProt accession no. Q15116) is set out below:

[SEQ ID NO: 30]
PGWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSGT
YLCGAISLAPKAQIKESLRAELRVTERRAEVPTAHPSPSPRPAGQFQTLV
VGVVGGLLGSLVLLVWVLAVICSRAARGTIGARRTGQPLKEDPSAVPVFS
VDYGELDFQWREKTPEPPVPCVPEQTEYATIVFPSGMGTSSPARRGSADG
PRSAQPLRPEDGHCSWPL.

Examples of MAbs that bind to human PD-1, and therefore of use in the present invention, are described in US Patent Publication Nos. US2003/0039653, US2004/0213795, US2006/0110383, US2007/0065427, US2007/0122378, US2012/237522, and International PCT Publication Nos. WO2004/072286, WO2006/121168, WO2006/133396, WO2007/005874, WO2008/083174, WO2008/156712, WO2009/024531, WO2009/014708, WO2009/114335, WO2010/027828, WO2010/027423, WO2010/036959, WO2010/029435, WO2010/029434, WO2010/063011, WO2010/089411, WO2011/066342, WO2011/110604, WO2011/110621, and WO2012/145493 (the entire contents of which is incorporated herein by reference). Specific MAbs that are useful for the purposes of the present invention include the anti-PD-1 MAbs nivolumab, pembrolizumab, and pidilizumab, as well as the humanized anti-PD-1 antibodies h409A11, h409A16, and h409A17 described in International Patent Publication No. WO2008/156712.

The anti-PD-1 antigen-binding molecules of the invention preferably bind to a region of the extracellular domain of PD-1. By way of example, the anti-PD-1 antigen-binding molecules may specifically bind to a region of the extracellular domain of human PD-1, which comprises one or both of the amino acid sequences SFVLNWYRMSPSNQTDKLAAFPEDR [SEQ ID NO:9] (i.e., residues 62 to 86 of the native PD-1 sequence set forth in SEQ ID NO:10) and SGTYLCGAISLAPKAQIKE [SEQ ID NO:11] (i.e., residues 118 to 136 of the native PD-1 sequence set forth in SEQ ID NO:10). In another example, the anti-PD-1 antigen-binding molecule binds to a region of the extracellular domain of human PD-1 that comprises the amino acid sequence NWYRMSPSNQTDKLAAFPEDRSQPGQDCRFRV [SEQ ID NO:12] (i.e., corresponding to residue 66 to 97 of the native human PD-1 sequence set forth in SEQ ID NO:10).

In certain embodiments, the anti-PD-1 antigen-binding molecule comprises the fully humanized IgG4 MAb nivolumab (as described in detail in U.S. Pat. No. 8,008,449 (referred to as “5C4”), which is incorporated herein by reference in its entirety) or an antigen-binding fragment thereof. In representative examples of this type, the anti-PD-1 antigen-binding molecule comprises the CDR sequences as set forth in Table 2.

TABLE 2

| Heavy chain | | Light chain | |
|---|---|---|---|
| CDR1 | NSGMH | CDR1 | RASQSVSSYLA |
| | [SEQ ID NO: 24] | | [SEQ ID NO: 27] |
| CDR2 | VIWYDGSKRYYADSVKG | CDR2 | DASNRAT |
| | [SEQ ID NO: 25] | | [SEQ ID NO: 28] |
| CDR3 | NDDYW | CDR3 | QQSSNWPRT |
| | [SEQ ID NO: 26] | | [SEQ ID NO: 2P] |

In other specific embodiments, the anti-PD-1 antigen-binding molecule comprises a heavy chain amino acid sequence of nivolumab as set out for example below:

[SEQ ID NO: 30]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK;

or an antigen-binding fragment thereof, which comprises, consists or consists essentially of the amino acid sequence:

[SEQ ID NO: 31]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS.

In some of the same and other embodiments, the anti-PD-1 antigen-binding molecule may comprise the light chain amino acid sequence of nivolumab as set out for example below:

[SEQ ID NO: 32]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;

or an antigen-binding fragment thereof, which comprises, consists or consists essentially of the amino acid sequence:

[SEQ ID NO: 33]
EIVLTQSPATLSLSPGERATLSCRASQSVSSYLAWYQQKP
GQAPRLLIYDASNRATGIPARFSGSGSGTDFTLTISSLEP
EDFAVYYCQQSSNWPRTFGQGTKVEIK.

In alternate embodiments, the anti-PD-1 antigen-binding molecule comprises the humanized IgG4 MAb pembrolizumab or an antigen-binding fragment thereof. In non-limiting examples of this type, the anti-PD-1 antigen-binding molecule comprises the CDR sequences as set forth in Table 3.

TABLE 3

| Heavy chain | | Light chain | |
|---|---|---|---|
| CDR1 | NYYMY | CDR1 | RASKGVS |
| | [SEQ ID | | TSGYSYLH |
| | NO: 34] | | [SEQ ID |
| | | | NO: 37] |
| CDR2 | GINPSNGG | CDR2 | LASYLES |
| | TNFNEKFK | | [SEQ ID |
| | N | | NO: 38] |
| | [SEQ ID | | |
| | NO: 35] | | |
| CDR3 | RDYRFDM | CDR3 | QHSRDL |
| | GFDY | | PLT |
| | [SEQ ID | | [SEQ ID |
| | NO: 36] | | NO: 39] |

In some embodiments, the anti-PD-1 antigen-binding molecule competes with the MAb pembrolizumab for binding to PD-1.

In additional embodiments, the anti-PD-1 antigen-binding molecule comprises the heavy chain amino acid sequence of pembrolizumab as set out for example below:

[SEQ ID NO: 40]
QVQLVQSGVEVKKPGASVKVSCKASGYTFTNYYMY
WVRQAPGQGLEWMGGINPSNGGTNFNEKFKNRVTL
TTDSSTTTAYMELKSLQFDDTAVYYCARRDYRFDM
GFDYWGQGTTVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPPCPAPEFLGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLGK;

or an antigen-binding fragment thereof, which comprises, consists or consists essentially of the amino acid sequence:

[SEQ ID NO: 41]
QVQLVQSGVEVKKPGASVKVSCKASGYTFTNYYMY
WVRQAPGQGLEWMGGINPSNGGTNFNEKFKNRVTL
TTDSSTTTAYMELKSLQFDDTAVYYCARRDYRFDM
GFDYWGQGTTVTVSS.

Similarly, the anti-PD-1 antigen-binding molecule may comprise a light chain amino acid sequence of pembrolizumab as set out for example below:

[SEQ ID NO: 42]
EIVLTQSPATLSLSPGERATLSCRASKGVSTSGYS
YLHWYQQKPGQAPRLLIYLASYLESGVPARFSGSG
SGTDFTLTISSLEPEDFAVYYCQHSRDLPLTFGGG
TKVEIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC;

or an antigen-binding fragment thereof, which comprises, consists or consists essentially of the amino acid sequence:

[SEQ ID NO: 43]
EIVLTQSPATLSLSPGERATLSCRASKGVSTSGYS
YLHWYQQKPGQAPRLLIYLASYLESGVPARFSGSG
SGTDFTLTISSLEPEDFAVYYCQHSRDLPLTFGGG
TKVEIK.

In yet other embodiments of this type, the anti-PD-1 antigen-binding molecule comprises the MAb pidilizumab or an antigen-binding fragment thereof. In some related embodiments, the anti-PD-1 antigen-binding molecule comprises CDR sequences as set forth in Table 4.

TABLE 4

| Heavy chain | | Light chain | |
|---|---|---|---|
| CDR1 | NYGMN | CDR1 | SARSS |
| | [SEQ ID | | VSYMH |
| | NO: 44] | | [SEQ ID |
| | | | NO: 47] |
| CDR2 | WINTDSG | CDR2 | RTSNLAS |
| | ESTYAEE | | [SEQ ID |
| | FKG | | NO: 48] |
| | [SEQ ID | | |
| | NO: 45] | | |
| CDR3 | VGYDALDY | CDR3 | QQRSSF |
| | [SEQ ID | | PLT |
| | NO: 46] | | [SEQ ID |
| | | | NO: 49] |

In more specific embodiments, the anti-PD-1 antigen-binding molecule comprises a heavy chain amino acid sequence of pidilizumab as set forth below:

[SEQ ID NO: 50]
QVQLVQSGSELKKPGASVKISCKASGYTFTNYGMN
WVRQAPGQGLQWMGWINTDSGESTYAEEFKGRFVF
SLDTSVNTAYLQITSLTAEDTGMYFCVRVGYDALD
YWGQGTLVTVSSASTKGPSVFPLAPSSKSTSGGTA
ALGCLVKDYFPEPVTVSWNSGALTSGVHTFPAVLQ
SSGLYSLSSVVTVPSSSLGTQTYICNVNHKPSNTK
VDKRVEPKSCDKTHTCPPCPAPELLGGPSVFLFPP
KPKDTLMISRTPEVTCVVVDVSHEDPEVKFNWYVD
GVEVHNAKTKPREEQYNSTYRVVSVLTVLHQDWLN
GKEYKCKVSNKALPAPIEKTISKAKGQPREPQVYT
LPPSREEMTKNQVSLTCLVKGFYPSDIAVEWESNG
QPENNYKTTPPVLDSDGSFFLYSKLTVDKSRWQQG
NVFSCSVMHEALHNHYTQKSLSLSPGK;

or an antigen-binding fragment thereof, which comprises, consists or consists essentially of the amino acid sequence:

[SEQ ID NO: 51]
QVQLVQSGSELKKPGASVKISCKASGYTFTNYGMN
WVRQAPGQGLQWMGWINTDSGESTYAEEFKGRFVF
SLDTSVNTAYLQITSLTAEDTGMYFCVRVGYDALD
YWGQGTLVTVSS.

In some of the same and other embodiments, the anti-PD-1 antigen-binding molecule comprises the light chain amino acid sequence of pidilizumab as shown below:

[SEQ ID NO: 52]
EIVLTQSPSSLSASVGDRVTITCSARSSVSYMHWF
QQKPGKAPKLWIYRTSNLASGVPSRFSGSGSGTSY
CLTINSLQPEDFATYYCQQRSSFPLTFGGGTKLEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC,

or an antigen-binding fragment thereof, which comprises, consists or consists essentially of the amino acid sequence:

[SEQ ID NO: 53]
EIVLTQSPSSLSASVGDRVTITCSARSSVSYMHWF
QQKPGKAPKLWIYRTSNLASGVPSRFSGSGSGTSY
CLTINSLQPEDFATYYCQQRSSFPLTFGGGTKLEI
K.

Other suitable MAbs are described in the International Patent Publication No. WO2015/026634, which is hereby incorporated by reference herein in its entirety. These include MAbs, or antigen-binding fragments thereof, which comprise: (a) light chain CDRs with amino acid sequences: RASKSVSTSGFSYLH [SEQ ID NO:54], LASNLES [SEQ ID NO:55], and QHSWELPLT [SEQ ID NO:56] (CDR1, CDR2, and CDR3, respectively) and heavy chain CDRs with amino acid sequences SYYLY [SEQ ID NO:57], GVNPSNGGTNFSEKFKS [SEQ ID NO:58] and RDSNYDGGFDY [SEQ ID NO:59] (CDR1, CDR2, and CDR3, respectively); or (b) light chain CDRs with amino acid sequence RASKGVSTSGYSYLH [SEQ ID NO:60], LASYLES [SEQ ID NO:61], and QHSRDLPLT [SEQ ID NO:62] (CDR1, CDR2, and CDR3, respectively), and heavy chain CDRs with amino acid sequence NYYMY [SEQ ID NO:63], GINPSNGGTNFNEKFKN [SEQ ID NO:64], and RDYRFDMGFDY [SEQ ID NO:65] (CDR1, CDR2, and CDR3, respectively).

By way of an illustration, such MAbs may comprise (a) a heavy chain variable region comprising:

[SEQ ID NO: 66]
QVQLVQSGVEVKKPGASVKVSCKASGYTFTNYYMY
WVRQAPGQGLEWMGGINPSNGGTNFNEKFKNRVTL
TTDSSTTTAYMELKSLQFDDTAVYYCARRDYRFDM
GFDYWGQGTTVTVSS,

or a variant or antigen-binding fragment thereof; and

a light chain variable region comprising an amino acid sequence selected from:

[SEQ ID NO: 67]
EIVLTQSPATLSLSPGERATLSCRASKGVSTSGYS
YLHWYQQKPGQAPRLLIYLASYLESGVPARFSGSG
SGTDFTLTISSLEPEDFAVYYCQHSRDLPLTFGGG
TKVEIK,
[SEQ ID NO: 68]
IVLTQSPLSLPVTPGEPASISCRASKGVSTSGYSY
LHWYLQKPGQSPQLLIYLASYLESGVPDRFSGSGS
GTDFTLKISRVEAEDVGVYYCQHSRDLPLTFGQGT
KLEIK,
or
[SEQ ID NO: 69]
DIVMTQTPLSLPVTPGEPASISCRASKGVSTSGYS
YLHWYLQKPGQSPQLLIYLASYLESGVPDRFSGSG
SGTAFTLKISRVEAEDVGLYYCQHSRDLPLTFGQG
TKLEIK,

or a variant or antigen-binding fragment thereof.

In yet further exemplary embodiments the anti-PD-1 MAb may comprise the IgG1 heavy chain comprising:

[SEQ ID NO: 70]
QVQLVQSGVEVKKPGASVKVSCKASGYTFTNYYMY
WVRQAPGQGLEWMGGINPSNGGTNFNEKFKNRVTL
TTDSSTTTAYMELKSLQFDDTAVYYCARRDYRFDM
GFDYWGQGTTVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPPCPAPEFLGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLGK

or a variant or antigen-binding fragment thereof;

and a light chain comprising any one of:

[SEQ ID NO: 71]
EIVLTQSPATLSLSPGERATLSCRASKGVSTSGYS
YLHWYQQKPGQAPRLLIYLASYLESGVPARFSGSG
SGTDFTLTISSLEPEDFAVYYCQHSRDLPLTFGGG
TKVEIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC,
[SEQ ID NO: 72]
EIVLTQSPLSLPVTPGEPASISCRASKGVSTSGYS
YLHWYLQKPGQSPQLLIYLASYLESGVPDRFSGSG
SGTDFTLKISRVEAEDVGVYYCQHSRDLPLTFGQG
TKLEIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC
[SEQ ID NO: 73]
DIVMTQTPLSLPVTPGEPASISCRASKGVSTSGYS
YLHWYLQKPGQSPQLLIYLASYLESGVPDRFSGSG
SGTAFTLKISRVEAEDVGLYYCQHSRDLPLTFGQG
TKLEIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC,

or a variant or an antigen-binding fragment thereof.

In other embodiments, the ICM antagonist is a PD-L1 antagonist. Alternative names or synonyms for PD-L1 include PDCD1L1, PDL1, B7H1, 67-4, CD274, and 67-H. Generally, the PD-L1 antagonists specifically bind to the native amino acid sequence of human PD-L1 (UniProt accession no. Q9NZQ7) as set out below:

[SEQ ID NO: 14]
MRIFAVFIFMTYWHLLNAFTVTVPKDLYVVEYGSN
MTIECKFPVEKQLDLAALIVYWEMEDKNIIQFVHG
EEDLKVQHSSYRQRARLLKDQLSLGNAALQITDVK
LQDAGVYRCMISYGGADYKRITVKVNAPYNKINQR
ILVVDPVTSEHELTCQAEGYPKAEVIWTSSDHQVL
SGKTTTTNSKREEKLFNVTSTLRINTTTNEIFYCT
FRRLDPEENHTAELVIPELPLAHPPNERTHLVILG
AILLCLGVALTFIFRLRKGRMMDVKKCGIQDTNSK
KQSDTHLEET.

Suitably, the PD-L1 antagonist is an anti-PD-L1 antigen-binding molecule. By way of example, anti-PD-L1 antigen-binding molecules that are suitable for use with the present invention include the anti-PD-L1 MAbs durvalumab (MEDI4736), atezolizumab (Tecentriq), BMS-936559/MDX-1105, MSB0010718C, LY3300054, CA-170, GNS-1480, MPDL3280A, and avelumab. These and other anti-PD-L1 antibodies are described in International Publication Nos. WO2007/005874 and WO2010/077634, and U.S. Pat. Nos. 8,217,149, and 8,779,108, the entirety of each of which is incorporated herein by reference. Further anti-PD-L1 MAbs are described in International PCT Patent Publication No. WO2016/007,235, the entire contents of which is also incorporated herein by reference.

The anti-PD-L1 antigen-binding molecules suitably bind to a region of the extracellular domain of PD-L1. By way of illustration, the anti-PD-L1 antigen-binding molecules may specifically bind to a region of the extracellular domain of human PD-L1 that comprises the amino acid sequence SKKQSDTHLEET [SEQ ID NO:13] (i.e., residues 279 to 290 of the native PD-L1 sequence set forth in SEQ ID NO:14). In certain embodiments, the anti-PD-L1 antigen-binding molecule comprises the fully humanized IgG1 MAb durvalumab (as described with reference to “MEDI4736” in International PCT Publication No. WO2011/066389, and U.S. Patent Publication No 2013/034559, which are incorporated herein by reference in their entirety) or an antigen-binding fragment thereof. In representative embodiments of this type, the anti-PD-L1 antigen-binding molecule comprises the CDR sequences as set forth in Table 5.

TABLE 5

| Heavy chain | | Light chain | |
|---|---|---|---|
| CDR1 | RYWMS | CDR1 | RASQRV |
| | [SEQ ID | | SSSYLA |
| | NO: 74] | | [SEQ ID |
| | | | NO: 77] |
| CDR2 | NIKQDGSE | CDR2 | DASSRA |
| | KYYVDSVK | | TGIPD |
| | [SEQ ID | | [SEQ ID |
| | NO: 75] | | NO:78] |
| CDR3 | EGGWFGE | CDR3 | QQYGSL |
| | LAFDY | | PWT |
| | [SEQ ID | | [SEQ ID |
| | NO: 76] | | NO: 79] |

In more specific embodiments, the anti-PD-L1 antigen-binding molecule comprises the heavy chain amino acid sequence of durvalumab as set out for example below:

[SEQ ID NO: 80]
VQLVESGGGLVQPGGSLRLSCAASGFTFSRYWMSW
VRQAPGKGLEWVANIKQDGSEKYYVDSVKGRFTIS
RDNAKNSLYLQMNSLRAEDTAVYYCAREGGWFGEL
AFDYWGQGTLVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKRVEPKSCDKTHTCPPCPAPEFEGGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPASIEKTISKAKGQPREPQ
VYTLPPSREEMTKNQVSLTCLVKGFYPSDIAVEWE
SNGQPENNYKTTPPVLDSDGSFFLYSKLTVDKSRW
QQGNVFSCSVMHEALHNHYTQKSLSLSPG,

or an antigen-binding fragment thereof, which comprises, consists or consists essentially of the amino acid sequence:

[SEQ ID NO: 81]
VQLVESGGGLVQPGGSLRLSCAASGFTFSRYWMSWV
RQAPGKGLEWVANIKQDGSEKYYVDSVKGRFTISR
DNAKNSLYLQMNSLRAEDTAVYYCAREGGWFGELA
FDYWGQGTLVTVSS.

In some of the same and other embodiments, the anti-PD-L1 antigen-binding molecule may comprise the light chain amino acid sequence:

[SEQ ID NO: 82]
EIVLTQSPGTLSLSPGERATLSCRASQRVSSSYLA
WYQQKPGQAPRLLIYDASSRATGIPDRFSGSGSGT
DFTLTISRLEPEDFAVYYCQQYGSLPWTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC,

or an antigen-binding fragment thereof, which comprises, consists or consists essentially of the amino acid sequence:

[SEQ ID NO: 83]
EIVLTQSPGTLSLSPGERATLSCRASQRVSSSYLA
WYQQKPGQAPRLLIYDASSRATGIPDRFSGSGSGT
DFTLTISRLEPEDFAVYYCQQYGSLPWTFGQGTKV
EIK.

Alternatively, the anti-PD-L1 antigen-binding molecule competes for binding to PD-L1 with the MAb durvalumab.

In other embodiments, the anti-PD-L1 antigen-binding molecule comprises the fully humanized IgG1 MAb atezolizumab (as described in U.S. Pat. No. 8,217,148, the entire content of which is incorporated herein by reference) or an antigen-binding fragment thereof. In representative embodiments of this type, the anti-PD-L1 antigen-binding molecule comprises the CDR sequences as set forth in Table 6.

TABLE 6

| Heavy chain | | Light chain | |
|---|---|---|---|
| CDR1 | GFTFSX₁SWIH | CDR1 | RASQX₄X₅ |
| | [SEQ ID | | X₆TX₇X₈A |
| | NO: 84] | | [SEQ ID |
| | | | NO: 87] |
| CDR2 | AWIX₂PYGGS | CDR2 | SASX₉L |
| | X₃YYADSVKG | | X₁₀S |
| | [SEQ ID | | [SEQ ID |
| | NO: 85] | | NO: 88] |
| CDR3 | RHWPGGFDY | CDR3 | QQX₁₁X₁₂X₁₃ |
| | [SEQ ID | | X₁₄PX₁₅T |
| | NO: 86] | | [SEQ ID |
| | | | NO: 89] |

wherein X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, or F; X₁₂ is L, Y, or F; X₁₃ is Y, N, T, G, F or I; X₁₄ is H, V, P, T, or I; and X₁₅ is A, W, R, P, or T.

In more specific embodiments, the anti-PD-L1 antigen-binding molecule comprises the heavy chain amino acid sequence of atezolizumab as set forth for example below:

[SEQ ID NO: 90]
EVQLVESGGGLVQPGGSLRLSCAASGFTFSDSWIH
WVRQAPGKGLEWVAWISPYGGSTYYADSVKGRFTI
SADTSKNTAYLQMNSLRAEDTAVYYCARRHWPGGF
DYWGQGTLVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYASTYRVVSVLTVLHQDWL
NGKEYKCKVSNKALPAPIEKTISKAKGQPREPQVY
TLPPSREEMTKNQVSLTCLVKGFYPSDIAVEWESN
GQPENNYKTTPPVLDSDGSFFLYSKLTVDKSRWQQ
GNVFSCSVMHEALHNHYTQKSLSLSPGK,

or an antigen-binding fragment thereof, which comprises, consists or consists essentially of the amino acid sequence:

[SEQ ID NO: 91]
EVQLVESGGGLVQPGGSLRLSCAASGFTFSDSWIH
WVRQAPGKGLEWVAWISPYGGSTYYADSVKGRFTI
SADTSKNTAYLQMNSLRAEDTAVYYCARRHWPGGF
DYWGQGTLVTVSS.

In some of the same and other embodiments, the anti-PD-L1 antigen-binding molecule comprises the light chain amino acid sequence of atezolizumab as provided for example below:

[SEQ ID NO: 92]
DIQMTQSPSSLSASVGDRVTITCRASQDVSTAVAW
YQQKPGKAPKLLIYSASFLYSGVPSRFSGSGSGTD
FTLTISSLQPEDFATYYCQQYLYHPATFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC,

or an antigen-binding fragment thereof, which comprises, consists or consists essentially of the amino acid sequence:

[SEQ ID NO: 93]
DIQMTQSPSSLSASVGDRVTITCRASQDVSTAVAW
YQQKPGKAPKLLIYSASFLYSGVPSRFSGSGSGTD
FTLTISSLQPEDFATYYCQQYLYHPATFGQGTKVE
IK.

Alternatively, the anti-PD-L1 antigen-binding molecule competes for binding to PD-L1 with the MAb atezolizumab.

In other embodiments, the anti-PD-L1 antigen-binding molecule comprises the fully humanized IgG1 MAb avelumab (as described in U.S. Pat. No. 8,217,148, the entire contents of which is incorporated herein by reference) or an antigen-binding fragment thereof. In representative embodiments of this type, the anti-PD-L1 antigen-binding molecule comprises the CDR sequences as set forth in Table 7.

TABLE 7

| Heavy chain | | Light chain | |
|---|---|---|---|
| CDR1 | X₁YX₂MX₃ | CDR1 | TGTX₇X₈DV |
| | [SEQ ID | | GX₉YNYVS |
| | NO: 94] | | [SEQ ID |
| | | | NO: 97] |
| CDR2 | SIYPSGGX₄ | CDR2 | X₁₀VX₁₁ |
| | TFYADX₅VKG | | X₁₂RPS |
| | [SEQ ID | | [SEQ ID |
| | NO: 95] | | NO: 98] |
| CDR3 | IKLGTVTTV | CDR3 | SSX₁₃X₁₄ |
| | X₆Y | | X₁₅X₁₆ |
| | [SEQ ID | | X₁₇RV |
| | NO: 96] | | [SEQ ID |
| | | | NO: 99] |

wherein X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is I, N, or S; X₁₂ is D, H, or N; X₁₃ is F or Y; X₁₄ is N or S; X₁₆ is R, T, or S, X₁₅ is G or S, and X₁₇ is I or T.

In specific embodiments, the anti-PD-L1 antigen-binding molecule comprises the heavy chain amino acid sequence of avelumab as provided for example below:

[SEQ ID NO: 100]
EVQLLESGGGLVQPGGSLRLSCAASGFTFSSYIMM
WVRQAPGKGLEWVSSIYPSGGITFYADTVKGRFTI
SRDNSKNTLYLQMNSLRAEDTAVYYCARIKLGTVT
TVDYWGQGTLVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKKVEPKSCDKTHTCPPCPAPELLGGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPAPIEKTISKAKGQPREPQ
VYTLPPSRDELTKNQVSLTCLVKGFYPSDIAVEWE
SNGQPENNYKTTPPVLDSDGSFFLYSKLTVDKSRW
QQGNVFSCSVMHEALHNHYTQKSLSLSPGK,

or an antigen-binding fragment thereof, which comprises, consists or consists essentially of the amino acid sequence:

[SEQ ID NO: 101]
EVQLLESGGGLVQPGGSLRLSCAASGFTFSSYIMM
WVRQAPGKGLEWVSSIYPSGGITFYADTVKGRFTI
SRDNSKNTLYLQMNSLRAEDTAVYYCARIKLGTVT
TVDYWGQGTLVTVSS.

In some of the same and other embodiments, the anti-PD-L1 antigen-binding molecule comprises the light chain amino acid sequence of avelumab as set out for example below:

[SEQ ID NO: 102]
QSALTQPASVSGSPGQSITISCTGTSSDVGGYNYV
SWYQQHPGKAPKLMIYDVSNRPSGVSNRFSGSKSG
NTASLTISGLQAEDEADYYCSSYTSSSTRVFGTGT
KVTVLGQPKANPTVTLFPPSSEELQANKATLVCLI
SDFYPGAVTVAWKADGSPVKAGVETTKPSKQSNNK
YAASSYLSLTPEQWKSHRSYSCQVTHEGSTVEKTV
APTECS,

or an antigen-binding fragment thereof, which comprises, consists or consists essentially of the amino acid sequence:

[SEQ ID NO: 103]
QSALTQPASVSGSPGQSITISCTGTSSDVGGYNYV
SWYQQHPGKAPKLMIYDVSNRPSGVSNRFSGSKSG
NTASLTISGLQAEDEADYYCSSYTSSSTRVFGTGT
KVTVL.

Alternatively, the anti-PD-L1 antigen-binding molecule competes for binding to PD-L1 with the MAb avelumab.

In some embodiments, the ICM antagonist is an antagonist of CTLA4. Alternative names or synonyms for CTLA4 include ALPSS, CD, CD152, CELIAC3, CTLA-4, GRD4, GSE, IDDM12. Generally, the CTLA4 antagonists bind specifically to the mature amino acid sequence of human CTLA4 (UniProt accession no. P16410) as set out for example below:

[SEQ ID NO: 16]
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSDFLLWILAAVSSGLF
FYSFLLTAVSLSKMLKKRSPLTTGVYVKMPPTEPE
CEKQFQPYFIPIN.

Suitably, the CTLA4 antagonist is an anti-CTLA4 antigen-binding molecule. By way of example, anti-CTLA4 antigen-binding molecules that are suitable for use with the present invention include the anti-CTLA4 MAbs ipilimumab (BMS-734016, MDX-010, MDX-101) and tremelimumab (ticilimumab, CP-675,206).

The anti-CTLA4 antigen-binding molecules suitably bind to a region of the extracellular domain of CTLA4. By way of illustration, the anti-CTLA4 antigen-binding molecules may specifically bind to a region of the extracellular domain of human CTLA4 that comprises any one or more of the amino acid sequences YASPGKATEVRVTVLRQA [SEQ ID NO:15] (i.e., residues 26 to 42 of the native CTLA4 sequence set forth in SEQ ID NO:16), DSQVTEVCAATYMMGNELTFLDD [SEQ ID NO:17] (i.e., residues 43 to 65 of the native CTLA4 sequence set forth in SEQ ID NO:16), and VELMYPPPYYLGIG [SEQ ID NO:18] (i.e., residues 96 to 109 of the native CTLA4 sequence set forth in SEQ ID NO:16). Alternatively or in addition, the anti-CTLA4 antigen-binding molecules may specifically bind to a region of the extracellular domain of human CTLA4 that comprises any one or more and preferably all of the following residues of the mature form of CTLA4: K1, A2, M3, E33, R35, Q41, S44, Q45, V46, E48, L91, 193, K95, E97, M99, P102, P103, Y104, Y105, L106, 1108, N110.

In certain embodiments, the anti-CTLA4 antigen-binding molecule comprises the human IgG1 MAb ipilimumab (as described for example in International Publication WO2014/209804 and U.S. Patent Publication No 2015/0283234, the entire contents of which are incorporated herein by reference) or an antigen-binding fragment thereof. In representative embodiments of this type, the anti-CTA4 antigen-binding molecule comprises the CDR sequences as set forth in Table 8.

TABLE 8

| Heavy chain | | Light chain | |
|---|---|---|---|
| CDR1 | SYTMH | CDR1 | RASQSVG |
| | [SEQ ID | | SSYLA |
| | NO: 104] | | [SEQ ID |
| | | | NO: 107] |
| CDR2 | FISYDG | CDR2 | GAFSRAT |
| | NNKYVA | | [SEQ ID |
| | DSVKG | | NO: 108] |
| | [SEQ ID | | |
| | NO: 105] | | |
| CDR3 | TGWLGPF | CDR3 | QQYGS |
| | DY | | SPWT |
| | [SEQ ID | | [SEQ ID |
| | NO: 106] | | NO: 109] |

In more specific embodiments, the anti-CTLA4 antigen-binding molecule comprises the heavy chain amino acid sequence of ipilimumab as set out for example below:

[SEQ ID NO: 110]
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSRDELTKNQVSLTCLVKGFYPSDIAVEWESN
GQPENNYKTTPPVLDSDGSFFLYSKLTVDKSRWQQ
GNVFSCSVMHEALHNHYTQKSLSLSPGK,

or an antigen-binding fragment thereof, a non-limiting example of which comprises, consists or consists essentially of the amino acid sequence:

[SEQ ID NO: 111]
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSS.

In some of the same and other embodiments, the anti-CTLA4 antigen-binding molecule comprises the light chain amino acid sequence of ipilimumab as set out for example below:

[SEQ ID NO: 112]
EIVLTQSPGTLSLSPGERATLSCRASQSVGSSYLA
WYQQKPGQAPRLLIYGAFSRATGIPDRFSGSGSGT
DFTLTISRLEPEDFAVYYCQQYGSSPWTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC,

or an antigen-binding fragment thereof, a representative example of which comprises, consists or consists essentially of the amino acid sequence:

[SEQ ID NO: 113]
EIVLTQSPGTLSLSPGERATLSCRASQSVGSSYLA
WYQQKPGQAPRLLIYGAFSRATGIPDRFSGSGSGT
DFTLTISRLEPEDFAVYYCQQYGSSPWTFGQGTKV
EIK.

the anti-CTAL4 antigen-binding molecule comprises the human IgG2 MAb tremelimumab (as described for example in U.S. Patent Publication No 2009/0074787, the entire content of which is incorporated herein by reference) or an antigen-binding fragment thereof. In representative embodiments of this type, the anti-CTLA4 antigen-binding molecule comprises the CDR sequences as set forth in Table 9.

TABLE 9

| Heavy chain | | Light chain | |
|---|---|---|---|
| CDR1 | GFTFSSYGMH | CDR1 | RASQSINSYLD |
| | [SEQ ID NO: 114] | | [SEQ ID NO: 117] |
| CDR2 | VIWYDGSNKYYADSV | CDR2 | AASSLQS |
| | [SEQ ID NO: 115] | | [SEQ ID NO: 118] |
| CDR3 | DPRGATLYYYYYGMDV | CDR3 | QQYYSTPFT |
| | [SEQ ID NO: 116] | | [SEQ ID NO: 119] |

In more specific embodiments, the anti-CTLA4 antigen-binding molecule comprises the heavy chain amino acid sequence of tremelimumab as set out for example below:

[SEQ ID NO: 120]
QVQLVESGGGVVQPGRSLRLSCAASGFTFSSYGMH
WVRQAPGKGLEWVAVIWYDGSNKYYADSVKGRFTI
SRDNSKNTLYIQMNSLRAEDTAVYYCARDPRGATL
YYYYYGMDVWGQGTTVTVSSASTKGPSVFPLAPCS
RSTSESTAALGCLVKDYFPEPVTVSWNSGALTSGV
HTFPAVLQSSGLYSLSSVVTVPSSNFGTQTYTCNV
DHKPSNTKVDKTVERKCCVECPPCPAPPVAGPSVF
LFPPKPKDTLMISRTPEVTCVVVDVSHEDPEVQFN
WYVDGVEVHNAKTKPREEQFNSTFRVVSVLTVVHQ
DWLNGKEYKCKVSNKGLPAPIEKTISKTKGQPREP
QVYTLPPSREEMTKNQVSLTCLVKGFYPSDIAVEW
ESNGQPENNYKTTPPMLDSDGSFFLYSKLTVDKSR
WQQGNVFSCSVMHEALHNHYTQKSLSLSPGK,

or an antigen-binding fragment thereof, a non-limiting example of which comprises, consists or consists essentially of the amino acid sequence:

[SEQ ID NO: 121]
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSS.

In some of the same and other embodiments, the anti-CTLA4 antigen-binding molecule comprises the light chain amino acid sequence of tremelimumab as set out for example below:

[SEQ ID NO: 122]
DIQMTQSPSSLSASVGDRVTITCRASQSINSYLDW
YQQKPGKAPKLLIYAASSLQSGVPSRFSGSGSGTD
FTLTISSLQPEDFATYYCQQYYSTPFTFGPGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC,

or an antigen-binding fragment thereof, a representative example of which comprises, consists or consists essentially of the amino acid sequence:

[SEQ ID NO: 123]
DIQMTQSPSSLSASVGDRVTITCRASQSINSYLDW
YQQKPGKAPKLLIYAASSLQSGVPSRFSGSGSGTD
FTLTISSLQPEDFATYYCQQYYSTPFTFGPGTKVE
IK.

In other embodiments, the anti-ICM antigen-binding molecule is an anti-B7-H3 antigen-binding molecule. Generally, the 67-H3 antigen-binding molecules bind specifically to the native amino acid sequence of human 67-H3 (UniProt accession no. Q5ZPR3) as set out for example below:

[SEQ ID NO: 124]
MLRRRGSPGMGVHVGAALGALWFCLTGALEVQVPE
DPVVALVGTDATLCCSFSPEPGFSLAQLNLIWQLT
DTKQLVHSFAEGQDQGSAYANRTALFPDLLAQGNA
SLRLQRVRVADEGSFTCFVSIRDFGSAAVSLQVAA
PYSKPSMTLEPNKDLRPGDTVTITCSSYQGYPEAE
VFWQDGQGVPLTGNVTTSQMANEQGLFDVHSILRV
VLGANGTYSCLVRNPVLQQDAHSSVTITPQRSPTG
AVEVQVPEDPVVALVGTDATLRCSFSPEPGFSLAQ
LNLIWQLTDTKQLVHSFTEGRDQGSAYANRTALFP
DLLAQGNASLRLQRVRVADEGSFTCFVSIRDFGSA
AVSLQVAAPYSKPSMTLEPNKDLRPGDTVTITCSS
YRGYPEAEVFWQDGQGVPLTGNVTTSQMANEQGLF
DVHSVLRVVLGANGTYSCLVRNPVLQQDAHGSVTI
TGQPMTFPPEALWVTVGLSVCLIALLVALAFVCWR
KIKQSCEEENAGAEDQDGEGEGSKTALQPLKHSDS
KEDDGQEIA.

Suitably, an anti-B7-H3 antigen-binding molecule suitable for use with the present invention is the MAb enoblituzumab or an antigen-binding fragment thereof. In some embodiments the anti-B7-H3 antigen-binding molecule comprises CDR sequences as set forth in Table 10.

TABLE 10

| Heavy chain | | Light chain | |
|---|---|---|---|
| CDR1 | FGMH | CDR1 | KASQNVDTNVA |
| | [SEQ ID NO: 125] | | [SEQ ID NO: 128] |
| CDR2 | YISSDSSAIYYADTVK | CDR2 | SASYRYS |
| | [SEQ ID NO: 126] | | [SEQ ID NO: 129] |
| CDR3 | GRENIYYGSRLDY | CDR3 | QQYNNYPFT |
| | [SEQ ID NO: 127] | | [SEQ ID NO: 130] |

In more specific embodiments, the anti-B7-H3 antigen-binding molecule comprises the heavy chain amino acid sequence of enoblituzumab as set out for example below:

[SEQ ID NO: 131]
VQLVESGGGLVQPGGSLRLSCAASGFTFSSFGMHW
VRQAPGKGLEWVAYISSDSSAIYYADTVKGRFTIS
RDNAKNSLYLQMNSLRDEDTAVYYCGRGRENIYYG
SRLDYWGQGTTVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKP
SNTKVDKRVEPKSCDKTHTCPPCPAPELVGGPSVF
LLPPKPKDTLMISRTPEVTCVVVDVSHEDPEVKFN
WYVDGVEVHNAKTKPPEEQYNSTLRVVSVLTVLHQ
DWLNGKEYKCKVSNKALPAPIEKTISKAKGQPREP
QVYTLPPSREEMTKNQVSLTCLVKGFYPSDIAVEW
ESNGQPENNYKTTPLVLDSDGSFFLYSKLTVDKSR
WQQGNVFSCSVMHEALHNHYTQKSLSLSPGK,

or an antigen-binding fragment thereof, a representative example of which comprises, consists or consists essentially of the amino acid sequence:

[SEQ ID NO: 132]
VQLVESGGGLVQPGGSLRLSCAASGFTFSSFGMHWVRQAPGKGLEWVAYI
SSDSSAIYYADTVKGRFTISRDNAKNSLYLQMNSLRDEDTAVYYCGRGRE
NIYYGSRLDYWGQGTTVTVSS.

In some of the same and other embodiments, the anti-B7-H3 antigen-binding molecules comprise the light chain amino acid sequence of enoblituzumab as provided for example below.

[SEQ ID NO: 133]
DIQLTQSPSFLSASVGDRVTITCKASQNVDTNVAWYQQKPGKAPKALIYS
ASYRYSGVPSRFSGSGSGTDFTLTISSLQPEDFATYYCQQYNNYPFTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,

or an antigen-binding fragment thereof, a representative example of which comprises, consists or consists essentially of the amino acid sequence:

[SEQ ID NO: 134]
DIQLTQSPSFLSASVGDRVTITCKASQNVDTNVAWYQQKPGKAPKALIYS
ASYRYSGVPSRFSGSGSGTDFTLTISSLQPEDFATYYCQQYNNYPFTFGQ
GTKLEIK.

In some alternative embodiments, the anti-B7-H3 antigen-binding molecule competes for binding to 67-H3 with the MAb enoblituzumab.

In some embodiments, the anti-ICM antigen-binding molecule is an anti-KIR antigen-binding molecule. In preferred embodiments of this type, the anti-KIR antigen-binding molecule blocks the interaction between KIR2-DL-1, -2, and -3 and their ligands. The mature amino acid sequence of a human KIR, i.e., KIR2-DL1 (UniProt accession no. P43626) is provided for example below:

[SEQ ID NO: 135]
HEGVHRKPSLLAHPGPLVKSEETVILQCWSDVMFEHFLLHREGMFNDTLR
LIGEHHDGVSKANFSISRMTQDLAGTYRCYGSVTHSPYQVSAPSDPLDIV
IIGLYEKPSLSAQPGPTVLAGENVTLSCSSRSSYDMYHLSREGEAHERRL
PAGPKVNGTFQADFPLGPATHGGTYRCFGSFHDSPYEWSKSSDPLLVSVT
GNPSNSWPSPTEPSSKTGNPRHLHILIGTSVVIILFILLFFLLHRWCSNK
KNAAVMDQESAGNRTANSEDSDEQDPQEVTYTQLNHCVFTQRKITRPSQR
PKTPPTDIIVYTELPNAESRSKVVSCP.

Anti-KIR antigen-binding molecules that are suitable for use in the invention can be generated using methods well known in the art. Alternatively, art-recognized KIR antigen-binding molecules can be used. For example, the anti-KIR antigen-binding molecule comprises the fully humanized MAb lirilumab or an antigen-binding fragment thereof as described for example in WO2014/066532, the entire content of which is hereby incorporated herein in its entirety. Suitably, the anti-KIR antigen-binding molecule comprises the CDR regions as set forth in Table 11.

TABLE 11

| Heavy chain | | Light chain | |
|---|---|---|---|
| CDR1 | FYAIS [SEQ ID NO: | CDR1 | RASQSVSSYLA [SEQ ID |
| | 136] | | NO: 139] |
| CDR2 | GFIPIFGAANYAQKFQ | CDR2 | DASNRAT [SEQ ID NO: |
| | [SEQ ID NO: 137] | | 140] |
| CDR3 | IPSGSYYYDYDMDV | CDR3 | QQRSNWMYT [SEQ ID |
| | [SEQ ID NO: 138] | | NO: 141] |

In representative embodiments of this type, the anti-KIR antigen-binding molecule may comprise the heavy chain variable domain amino acid sequence of lirilumab, as set out for example below:

[SEQ ID NO: 142]
QVQLVQSGAEVKKPGSSVKVSCKASGGTFSFYAISWVRQAPGQGLEWMGG
FIPIFGAANYAQKFQGRVTITADESTSTAYMELSSLRSDDTAVYYCARIP
SGSYYYDYDMDVWGQGTTVTVSSASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK,

or an antigen-binding fragment thereof, a representative example of which comprises, consists or consists essentially of the amino acid sequence:

[SEQ ID NO: 143]
QVQLVQSGAEVKKPGSSVKVSCKASGGTFSFYAISWVRQAPGQGLEWMGG
FIPIFGAANYAQKFQGRVTITADESTSTAYMELSSLRSDDTAVYYCARIP
SGSYYYDYDMDVWGQGTTVTVSS.

In some of the same and other embodiments, the anti-KIR antigen-binding molecule may comprise the light chain variable domain amino acid sequence of lirilumab, as set out for example below:

[SEQ ID NO: 144]
EIVLTQSPVTLSLSPGERATLSCRASQSVSSYLAWYQQKPGQAPRLLIYD
ASNRATGIPARFSGSGSGTDFTLTISSLEPEDFAVYYCQQRSNWMYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,

or an antigen-binding fragment thereof, a representative example of which comprises, consists or consists essentially of the amino acid sequence:

[SEQ ID NO: 145]
EIVLTQSPVTLSLSPGERATLSCRASQSVSSYLAWYQQKPGQAPRLLIYD
ASNRATGIPARFSGSGSGTDFTLTISSLEPEDFAVYYCQQRSNWMYTFGQ
GTKLEIKRT.

In alternative embodiments, the anti-ICM antigen-binding molecule is an anti-LAG-3 antigen-binding molecule. LAG-3 is a 503 amino acid type I transmembrane protein, with four extracellular Ig-like domains. LAG-3 is expressed on activated T-cells, NK cells, B-cells, and plasmacytoid DCs. The representative mature amino acid sequence of human LAG-3 (UniProt accession no. P18627), is set out below:

[SEQ ID NO: 146]
LQPGAEVPVVWAQEGAPAQLPCSPTIPLQDLSLLRRAGVTWQHQPDSGPP
AAAPGHPLAPGPHPAAPSSWGPRPRRYTVLSVGPGGLRSGRLPLQPRVQL
DERGRQRGDFSLWLRPARRADAGEYRAAVHLRDRALSCRLRLRLGQASMT
ASPPGSLRASDWVILNCSFSRPDRPASVHWFRNRGQGRVPVRESPHHHLA
ESFLFLPQVSPM DSGPWGCILTYRDGFNVSIMYNLTVLGLEPPTPLTVY
AGAGSRVGLPCRLPAGVGTRSFLTAKWTPPGGGPDLLVTGDNGDFTLRLE
DVSQAQAGTYTCHIHLQEQQLNATVTLAIITVTPKSFGSPGSLGKLLCEV
TPVSGQERFVWSSLDTPSQRSFSGPWLEAQEAQLLSQPWQCQLYQGERLL
GAAVYFTELSSPGAQRSGRAPGALPAGHLLLFLILGVLSLLLLVTGAFGF
HLWRRQWRPRRFSALEQGIHPPQAQSKIEELEQEPEPEPEPEPEPEPEPE
PEQL.

In some embodiments, the anti-LAG-3 antigen-binding molecule is suitably the anti-LAG3 humanized MAb, BMS-986016. Other anti-LAG-3 antibodies are described in U.S. Patent Publication No. 2011/0150892 and International PCT Publication Nos. WO2010/019570 and WO2014/008218, each of which is incorporated herein by reference in their entirety.

In some embodiments, the anti-LAG-3 antigen-binding molecules comprise the CDR sequences set forth in Table 12.

TABLE 12

| Heavy chain | | Light chain | |
|---|---|---|---|
| CDR1 | DYYWN [SEQ ID NO: 147] | CDR1 | RASQSISSYLA |
| | | | [SEQ ID NO: 150] |
| CDR2 | EINHRGSTNSNPSLKS | CDR2 | DASNRAT [SEQ ID |
| | [SEQ ID NO: 148] | | NO: 151] |
| CDR3 | GYSDYEYNWFDP [SEQ ID | CDR3 | QQRSNWPLT |
| | NO: 149] | | [SEQ ID NO: 152] |

The anti-LAG-3 antigen-binding molecules suitably comprise the MAb BMS-986016 or an antigen-binding fragment thereof. More specifically, in some embodiments, the anti-LAG-3 antigen-binding molecule has the heavy chain amino acid sequence of BMS-986016 as set out for example below:

[SEQ ID NO: 153]
QVQLQQWGAGLLKPSETLSLTCAVYGGSFSDYYWNWIRQPPGKGLEWIGE
INHRGSTNSNPSLKSRVTLSLDTSKNQFSLKLRSVTAADTAVYYCAFGYS
DYEYNWFDPWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK,

or an antigen-binding fragment thereof, a representative example of which comprises, consists or consists essentially of the amino acid sequence:

[SEQ ID NO: 154]
QVQLQQWGAGLLKPSETLSLTCAVYGGSFSDYYWNWIRQPPGKGLEWIGE
INHRGSTNSNPSLKSRVTLSLDTSKNQFSLKLRSVTAADTAVYYCAFGYS
DYEYNWFDPWGQGTLVTVSS.

Similarly, the anti-LAG-3 antigen-binding molecules may comprise a light chain amino acid sequence of BMS-986016 as set forth in SEQ ID NO:45 and provided below, of an antigen-binging fragment thereof:

[SEQ ID NO: 156]
EIVLTQSPATLSLSPGERATLSCRASQSISSYLAWYQQKPGQAPRLLIYD
ASNRATGIPARFSGSGSGTDFTLTISSLEPEDFAVYYCQQRSNWPLTFGQ
GTNLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

or an antigen-binding fragment thereof, a representative example of which comprises, consists or consists essentially of the amino acid sequence:

[SEQ ID NO: 157]
EIVLTQSPATLSLSPGERATLSCRASQSISSYLAWYQQKPGQAPRLLIYD
ASNRATGIPARFSGSGSGTDFTLTISSLEPEDFAVYYCQQRSNWPLTFGQ
GTNLEIK.

Any suitable anti-AMA antigen-binding molecule that can be used in therapy is also contemplated for use in combination with the RANK antagonist antigen-binding molecules of the present invention.

### 5. Multispecific Antigen-Binding Molecules

In some embodiments in which the RANK antagonist antigen-binding molecule and anti-ICM or anti-AMA antigen-binding molecule(s) are provided in the same composition, they are conjugated together in the form of a multi-specific antigen-binding molecule.

Representative examples of multi-specific antigen-binding molecules include tandem scFv (taFv or scFv₂), diabody, dAb₂/V_{H}H₂, knobs-into-holes derivatives, SEED-IgG, heteroFc-scFv, Fab-scFv, scFv-Jun/Fos, Fab′-Jun/Fos, tribody, DNL-F(ab)₃, scFv₃-C_{H1}/CL, Fab-scFv₂, IgG-scFab, IgG-scFv, scFv-IgG, scFv₂-Fc, F(ab′)₂-scFv₂, scDB-Fc, scDb-C_{H}3, db-Fc, scFv₂-H/L, DVD-Ig, tandAb, scFv-dhlx-scFv, dAb₂-IgG, dAb-IgG, dAb-Fc-dAb, and combinations thereof. In specific embodiments, the synthetic or recombinant antigen-binding molecules are selected from IgG-like antibodies (e.g., triomab/quadroma, Trion Pharma/Fresenius Biotech; knobs-into-holes, Genentech; CrossMAbs, Roche; electrostatically matched antibodies, AMGEN; LUZ-Y, Genentech; strand exchange engineered domain (SEED) body, EMD Serono; biolonic, Merus; and Fab-exchanged antibodies, Genmab), symmetric IgG-like antibodies (e.g., dual targeting (DT)-Ig, GSK/Domantis; two-in-one antibody, Genentech; crosslinked MAbs, karmanos cancer center; MAb2, F-star; and Coy X-body, Coy X/Pfizer), IgG fusions (e.g., dual variable domain (DVD)-Ig, Abbott; IgG-like bispecific antibodies, Eli Lilly; Ts2Ab, Medimmune/AZ; BsAb, ZymoGenetics; HERCULES, Biogen Idec; TvAb, Roche) Fc fusions (e.g., ScFv/Fc fusions, Academic Institution; SCORPION, Emergent BioSolutions/Trubion, ZymoGenetics/BMS; dual affinity retargeting technology (Fc-DART), MacroGenics; dual (ScFv)₂-Fab, National Research Center for Antibody Medicine) Fab fusions (e.g., F(ab)₂, Medarex/AMGEN; dual-action or Bis-Fab, Genentech; Dock-and-Lock (DNL), ImmunoMedics; bivalent bispecific, Biotechnol; and Fab-Fv, UCB-Celltech), ScFv- and diabody-based antibodies (e.g., bispecific T cell engagers (BiTEs), Micromet; tandem diabodies (Tandab), Affimed; DARTs, MacroGenics; Single-chain diabody, Academic; TCR-like antibodies, AIT, Receptor Logics; human serum albumin ScFv fusion, Merrimack; and COMBODIES, Epigen Biotech), IgG/non-IgG fusions (e.g., immunocytokins, EMDSerono, Philogen, ImmunGene, ImmunoMedics; superantigen fusion protein, Active Biotech; and immune mobilizing mTCR Against Cancer, ImmTAC) and oligoclonal antibodies (e.g., Symphogen and Merus).

Other non-limiting examples of multi-specific antigen-binding molecules include a Fabs-in-tandem immunoglobulins (FIT-Ig) (Gong et al., 2017. MAbs. 9(7):1118-1128. doi: 10.1080/19420862.2017.1345401. Epub 2017 Jul. 10. PubMedPMID: 28692328; PubMed Central PMCID: PMC5627593), and are capable of binding two or more antigens. In the design of a FIT-Ig molecule, the two Fab domains from parental mAbs are fused directly in tandem in a crisscross orientation. The three fragments, when co-expressed in mammalian cells, assemble to form a tetravalent multi-specific FIT-Ig molecule. For instance, a bispecific binding protein could be constructed as a FIT-Ig using two parental monoclonal antibodies, mAb A (which binds to antigen A), and mAb B (which binds to antigen B). In the design of a FIT-Ig molecule, the two Fab domains from parental mAbs are fused directly in tandem in a crisscross orientation. The three fragments, when co-expressed in mammalian cells, assemble to form a tetravalent multi-specific FIT-Ig molecule. In representative embodiments, an FIT-Ig provides multi-specific antigen-binding molecules for antagonizing RANK and at least one ICM or at least one AMA. These multi-specific antigen-binding molecules generally comprise, consist or consist essentially of an antibody or antigen-binding fragment constructed as a FIT-Ig molecule thereof that binds specifically to and antagonize RANK and for a respective ICM or AMA, an antibody or antigen-binding fragment thereof that binds specifically to that ICM or AMA. The at least one anti-ICM antibody or antigen-binding fragment is suitably selected from an anti-PD-1 antibody or antigen-binding fragment, an anti-PD-L1 antibody or antigen-binding fragment, or an anti-CTLA-4 antibody or antigen-binding fragment and incorporated into a FIT-Ig molecule. In some embodiments in which the multi-specific antigen-binding molecule antagonizes PD-1, the multi-specific antigen-binding molecule comprises an anti-PD-1 antibody or antigen-binding fragment thereof. In some embodiments in which the multi-specific antigen-binding molecule antagonizes PD-L1, the multi-specific antigen-binding molecule comprises an anti-PD-L1 antibody or antigen-binding fragment thereof. In some embodiments in which the multi-specific antigen-binding molecule antagonizes CTLA4, the multi-specific antigen-binding molecule comprises an anti-CTLA4 antibody or antigen-binding fragment thereof.

Additionally, the at least one anti-AMA antibody or antigen-binding fragment is suitably selected from an anti-PD-L1 antibody or antigen-binding fragment, an anti-CD206 antibody or antigen-binding fragment, an anti-CD103 antibody or antigen-binding fragment, an anti-CD200 antibody or antigen-binding fragment, an anti-CD200 antibody or antigen-binding fragment, an anti-Gal9 antibody or antigen-binding fragment, an anti-HVEM antibody or antigen-binding fragment, an anti-CD38 antibody or antigen-binding fragment, an anti-CD163 antibody or antigen-binding fragment, or an anti-MARCO antibody or antigen-binding fragment and incorporated into a FIT-Ig molecule. Thus, in some embodiments in which the multi-specific antigen-binding molecule antagonizes CD206, the multi-specific antigen-binding molecule comprises an anti-CD206 antibody or antigen-binding fragment thereof. In some embodiments in which the multi-specific antigen-binding molecule antagonizes PD-L1, the multi-specific antigen-binding molecule comprises an anti-PD-L1 antibody or antigen-binding fragment thereof. In some embodiments in which the multi-specific antigen-binding molecule antagonizes CD103, the multi-specific antigen-binding molecule comprises an anti-CD103 antibody or antigen-binding fragment thereof. In some embodiments in which the multi-specific antigen-binding molecule antagonizes CD200, the multi-specific antigen-binding molecule comprises an anti-CD200 antibody or antigen-binding fragment thereof. In some embodiments in which the multi-specific antigen-binding molecule antagonizes HVEM, the multi-specific antigen-binding molecule comprises an anti-HVEM antibody or antigen-binding fragment thereof. In some embodiments in which the multi-specific antigen-binding molecule antagonizes CD38, the multi-specific antigen-binding molecule comprises an anti-CD38 antibody or antigen-binding fragment thereof. In some embodiments in which the multi-specific antigen-binding molecule antagonizes CD163, the multi-specific antigen-binding molecule comprises an anti-CD163 antibody or antigen-binding fragment thereof. In some embodiments in which the multi-specific antigen-binding molecule antagonizes MARCO, the multi-specific antigen-binding molecule comprises an anti-MARCO antibody or antigen-binding fragment thereof.

In certain embodiments, an antigen-binding molecule having a first antigen binding specificity can be functionally linked (e.g., by chemical coupling, genetic fusion, noncovalent association or otherwise) to one or more other molecular entities, such as another antigen-binding molecule having a second antigen-binding specificity to produce a bispecific antigen-binding molecule. Specific exemplary multispecific formats that can be used in the context of the present invention include, without limitation, single-chain diabody (scDb), tandem scDb (Tandab), linear dimeric scDb (LD-scDb), circular dimeric scDb (CD-scDb), bispecific T-cell engager (BITE; tandem di-scFv), disulfide-stabilized Fv fragment (Brinkmann et al., Proc Natl Acad Sci USA. 1993; 90: 7538-7542), tandem tri-scFv, tribody, bispecific Fab₂, di-miniantibody, tetrabody, scFv-Fc-scFv fusion, di-diabody, DVD-Ig, IgG-scFab, scFab-dsscFv, Fv2-Fc, IgG-scFv fusions, such as bsAb (scFv linked to C-terminus of light chain), Bs1Ab (scFv linked to N-terminus of light chain), Bs2Ab (scFv linked to N-terminus of heavy chain), Bs3Ab (scFv linked to C-terminus of heavy chain), Ts1Ab (scFv linked to N-terminus of both heavy chain and light chain), Ts2Ab (dsscFv linked to C-terminus of heavy chain), and Knob-into-Holes (KiHs) (bispecific IgGs prepared by the KiH technology) SEED technology (SEED-IgG) and DuoBodies (bispecific IgGs prepared by the DuoBody technology), a VH and a VL domain, each fused to one C-terminus of the two different heavy chains of a KiHs or DuoBody such that one functional Fv domain is formed. Particularly suitable for use herein is a single-chain diabody (scDb), in particular a bispecific monomeric scDb. For reviews discussing and presenting various multispecific constructs see, for example, Chan Carter, Nature Reviews Immunology 10 (2010) 301-316; Klein et al., MAbs 4(2012) 1-11; Schubert et al., Antibodies 1 (2012) 2-18; Byrne et al., Trends in Biotechnology 31 (2013) 621; Metz et al., Protein Engineering Design & Selection 25(2012) 571-580), and references cited therein.

In specific embodiments, the present invention provides bispecific antigen-binding molecules comprising a first antigen-binding molecule (e.g., an antibody or antigen-binding fragment) that binds specifically to and antagonizes RANK, and a second antigen-binding molecule (e.g., an antibody or antigen-binding fragment) that binds specifically to an ICM. The bispecific antigen-binding molecules suitably comprise any of the antigen-binding molecules described in detail above and elsewhere herein.

By way of illustration, the first antigen-binding molecule is a RANK antagonist antigen-binding molecule described herein, and the second antigen-binding molecule may bind specifically to a region of human PD-1, and preferably to a region of the extracellular domain of human PD-1.

Non-limiting examples of these embodiments include the first antigen-binding molecule comprising CDR sequences as set forth in Table 1. The second antigen-binding molecule suitably comprises the CDR sequences as set forth in any one of Tables 2-4. In specific examples of this type, the second antigen-binding molecule may comprises at least an antigen-binding fragment of any one of the MAbs selected from nivolumab, pembrolizumab, and pidilizumab.

In other embodiments, the second antigen-binding molecule binds specifically to a region of human PD-L1, and preferably to a region of the extracellular domain of human PD-L1. Thus, in some embodiments, the second antigen-binding molecule binds specifically to a region of PD-L1 and comprises the CDR sequences set forth in any one of Tables 5-7. In specific examples of this type, the second antigen-binding molecule may comprise at least an antigen-binding fragment of any one of the MAbs selected from durvalumab, atezolizumab, and avelumab.

In still other embodiments, the second antigen-binding molecule binds specifically to a region of human CTLA4. Thus, in some embodiments, the second antigen-binding molecule binds specifically to human CTLA4 and comprises the CDR sequences set forth in any one of Tables 8-9. In specific examples of this type, the second antigen-binding molecule may comprise at least an antigen-binding fragment of any one of the MAbs selected from ipilimumab and tremelimumab.

In specific embodiments, the present invention provides bispecific antigen-binding molecules comprising a first antigen-binding molecule (e.g., an antibody or antigen-binding fragment) that binds specifically to and antagonizes RANK, and a second antigen-binding molecule (e.g., an antibody or antigen-binding fragment) that binds specifically to an AMA.

In representative examples of these embodiments, the first antigen-binding molecule is a RANK antagonist antigen-binding molecule described herein, and the second antigen-binding molecule may bind specifically to a region of human PD-L1, and preferably to a region of the extracellular domain of human PD-L1.

In other representative examples, the first antigen-binding molecule is a RANK antagonist antigen-binding molecule described herein, and the second antigen-binding molecule may bind specifically to a region of human CD206, and preferably to a region of the extracellular domain of human CD206.

In other representative examples, the first antigen-binding molecule is a RANK antagonist antigen-binding molecule described herein, and the second antigen-binding molecule may bind specifically to a region of human CD103, and preferably to a region of the extracellular domain of human CD103.

In still other representative examples, the first antigen-binding molecule is a RANK antagonist antigen-binding molecule described herein, and the second antigen-binding molecule may bind specifically to a region of human CD200, and preferably to a region of the extracellular domain of human CD200.

In other representative examples, the first antigen-binding molecule is a RANK antagonist antigen-binding molecule described herein, and the second antigen-binding molecule may bind specifically to a region of human Gal9, and preferably to a region of the extracellular domain of human Gal9.

In other representative examples, the first antigen-binding molecule is a RANK antagonist antigen-binding molecule described herein, and the second antigen-binding molecule may bind specifically to a region of human HVEM, and preferably to a region of the extracellular domain of human HVEM.

In further representative examples, the first antigen-binding molecule is a RANK antagonist antigen-binding molecule described herein, and the second antigen-binding molecule may bind specifically to a region of human CD38, and preferably to a region of the extracellular domain of human CD38.

In other representative examples, the first antigen-binding molecule is a RANK antagonist antigen-binding molecule described herein, and the second antigen-binding molecule may bind specifically to a region of human CD163, and preferably to a region of the extracellular domain of human CD163.

In still other representative examples, the first antigen-binding molecule is a RANK antagonist antigen-binding molecule described herein, and the second antigen-binding molecule may bind specifically to a region of human MARCO, and preferably to a region of the extracellular domain of human MARCO.

The present invention also provides multispecific constructs that comprise a RANK antagonist antigen-binding molecule and a plurality of ICM antagonist antigen-binding molecules that have specificity for two or more ICMs. In non-limiting examples, the plurality of ICM antagonist antigen-binding molecules have specificity for an ICM combination selected from (1) PD-1 and PD-L1, (2) PD-1 and CTLA4, (3) PD-L1 and CTLA4, and (4) PD-1, PD-L1 and CTLA4. The multispecific constructs may comprise any suitable antibody or antigen-binding fragment with specificity for a particular ICM combination, including the antibody or antigen-binding fragment disclosed herein.

The present invention further provides multispecific constructs that comprise a RANK antagonist antigen-binding molecule and a plurality of AMA antagonist antigen-binding molecules that have specificity for two or more AMAs. In non-limiting examples, the plurality of AMA antagonist antigen-binding molecules have specificity for an AMA combination selected from (1) PD-L1 and CD206, (2) PD-L1 and CD103, (3) PD-L1 and CD200, (4) PD-L1 and Gal9, (5) PD-L1 and HVEM, (6) PD-L1 and CD38, (7) PD-L1 and CD163, (8) PD-L1 and MARCO, (9) CD206 and CD103, (10) CD206 and CD200, (11) CD206 and Gal9, (12) CD206 and HVEM, (13) CD206 and CD38, (14) CD206 and CD163, (15) CD206 and MARCO, (16) CD103 and CD200, (17) CD103 and Gal9, (18) CD103 and HVEM, (19) CD103 and CD38, (20) CD103 and CD163, (21) CD103 and MARCO, (22) CD200 and Gal9, (23) CD200 and HVEM, (24) CD200 and CD38, (25) CD200 and CD163, (26) CD200 and MARCO, (27) Gal9 and HVEM, (28) Gal9 and CD38, (29) Gal9 and CD163, (30) Gal9 and MARCO, (31) HVEM and CD38, (32) HVEM and CD163, (33) HVEM and MARCO, (34) CD38 and CD163, (35) CD38 and MARCO, (36) CD163 and MARCO, (37) PD-L1, CD206 and CD103, (38) PD-L1, CD206 and CD200, (39) PD-L1, CD206 and Gal9, (40) PD-L1, CD206 and HVEM, (41) PD-L1, CD206 and CD38, (42) PD-L1, CD206 and CD163, (43) PD-L1, CD206 and MARCO, (44) CD206, CD103 and CD200, (45) CD206, CD103 and Gal9, (46) CD206, CD103 and HVEM, (47) CD206, CD103 and CD38, (48) CD206, CD103 and CD163, (49) CD206, CD103 and MARCO, (50) CD103, CD200 and Gal9, (51) CD103, CD200 and HVEM, (52) CD103, CD200 and CD38, (53) CD103, CD200 and CD163, (54) CD103, CD200 and MARCO, (55) CD200, Gal9 and HVEM, (56) CD200, Gal9 and CD38, (57) CD200, Gal9 and CD163, (58) CD200, Gal9 and MARCO, (59) Gal9, HVEM and CD38, (60) Gal9, HVEM and CD163, (61) Gal9, HVEM and MARCO, (62) HVEM, CD38 and CD163, (63) HVEM, CD38 and MARCO, (64) CD38, CD163 and MARCO. The multispecific constructs may comprise any suitable antibody or antigen-binding fragment with specificity for a particular ICM combination, including the antibody or antigen-binding fragment disclosed herein.

Multispecific antigen-binding molecules of the present invention can be generated by any number of methods well known in the art. Suitable methods include biological methods (e.g., somatic hybridization), genetic methods (e.g., the expression of a non-native DNA sequence encoding the desired antibody structure in an organism), chemical methods (e.g., chemical conjugation of two antibodies), or a combination thereof (see, Kontermann R E (ed.), Bispecific Antibodies, Springer Heidelberg Dordrecht London New York, 1-28 (2011)).

5.1 Chemical Methods of Producing Bispecific Antigen-Binding Molecules.

Chemically conjugated bispecific antigen-binding molecules arise from the chemical coupling of two existing antibodies or antibody fragments, such as those described above and elsewhere herein. Typical couplings include cross-linking two different full-length antibodies, cross-linking two different Fab′ fragments to produce a bispecific F(ab′)₂, and cross-linking a F(ab′)₂ fragment with a different Fab′ fragment to produce a bispecific F(ab′)₃. For chemical conjugation, oxidative re-association strategies can be used. Current methodologies include the use of the homo- or heterobifunctional cross-linking reagents (Id.).

Heterobifunctional cross-linking reagents have reactivity toward two distinct reactive groups on, for example, antibody molecules. Examples of heterobifunctional cross-linking reagents include SPDP (N-succinimidyl-3-(2-pyridyldithio)propionate), SATA (succinimidyl acetylthioacetate), SMCC (succinimidyl trans-4-(maleimidylmethyl)cyclohexane-1-carboxylate), EDAC (1-ethyl-3-(3-dimethylaminopropyl) carbodiimide), PEAS (N-((2-pyridyldithio)ethyl)-4-azidosalicylamide), ATFB-SE (4-azido-2,3,5,6-tetrafluorobenzoic acid, succinimidyl ester), benzophenone-4-maleimide, benzophenone-4-isothiocyanate, 4-benzoylbenzoic acid, succinimidyl ester, iodoacetamide azide, iodoacetamide alkyne, Click-iT maleimide DIBO alkyne, azido (PEO)4 propionic acid, succinimidyl ester, alkyne, succinimidyl ester, Click IT succinimidyl ester DIBO alkyne, Sulfo-SBED (sulfo-N-hydroxysuccinimidyl-2-(6-[biotinamido]-2-(p-azido benzamido)-hexanoamido)ethyl-1,3′-dithioproprionate), photoreactive amino acids (e.g., L-photo-leucine and L-photo-methionine), NHS-haloacetyl crosslinkers (e.g., sulfo-SIAB), SIAB, SBAP, SIA, NHS-maleimide crosslinkers (e.g., sulfo-SMCC), SM(PEG), series cross-linkers, SMCC, LC-SMCC, sulfo-EMCS, EMCS, sulfo-GMBS, GMBS, sulfo-KMUS, sulfo-MBS, MBS, Sulfo-SMPB, SMPB, AMAS, BMPS, SMPH, PEG12-SPDP, PEG4-SPDP, sulfo-LC-SPDP, LC-SPDP, SMPT, DCC (N,N′-Dicyclohexylcarbodiimide), EDC (1-Ethyl-3-(3-dimethylaminopropyl) carbodiimide), NHS (N-hydroxysuccinimide), sulfo-NHS (N-hydroxysulfosuccinimide), BMPH, EMCH, KMUH, MPBH, PDPH, and PMPI.

Homobifunctional cross-linking reagents have reactivity toward the same reactive group on a molecule, for example, an antibody. Examples of homobifunctional cross-linking reagents include DTNB (5,5′-dithiobis(2-nitrobenzoic acid), o-PDM (o-phenylenedimaleimide), DMA (dimethyl adipimidate), DMP (dimethyl pimelimidate), DMS (dimethyl suberimidate), DTBP (dithiobispropionimidate), BS(PEG)₅, BS(PEG)₉, BS³, BSOCOES, DSG, DSP, DSS, DST, DTSSP, EGS, sulfo-EGS, TSAT, DFDNB, BM(PEG), cross-linkers, BMB, BMDB, BMH, BMOE, DTME, and TMEA.

5.2 Biological Methods of Producing Bispecific Antigen-Binding Molecules

Somatic hybridization is the fusion of two distinct hybridoma (a fusion of B-cells that produce a specific antibody and myeloma cells) cell lines, producing a quadroma capable of generating two different antibody heavy chains (i.e., V_{H}A and V_{H}B) and light chains (i.e., V_{L}A and V_{LB}). (Kontermann, supra). These heavy and light chains combine randomly within the cell, resulting in bispecific antigen-binding molecules (e.g., a V_{H}A chain combined with a V_{L}A chain and a V_{H}B chain combined with a V_{L}B chain), as well as some non-functional (e.g., two V_{H}A chains combined with two V_{L}B chains) and monospecific (e.g., two V_{H}A chains combined with two V_{H}A chains) antigen-binding molecules. The bispecific antigen-binding molecules can then be purified using well established methods, for example, using two different affinity chromatography columns.

Similar to monospecific antigen-binding molecules, bispecific antigen-binding molecules may also contain an Fc region that elicits Fc-mediated effects downstream of antigen binding. These effects may be reduced by, for example, proteolytically cleaving the Fc region from the bispecific antibody by pepsin digestion, resulting in bispecific F(ab′)2 molecules (Id.).

5.3 Genetic Methods of Producing Multispecific Antigen-Binding Molecules

Multispecific antigen-binding molecules may also be generated by genetic means as well established in the art, e.g., in vitro expression of a plasmid containing a DNA sequence corresponding to the desired antibody structure (see, e.g., Kontermann, supra).

5.4 Diabodies

In some embodiments, the multispecific antigen-binding molecule is a diabody. Diabodies are composed of two separate polypeptide chains from, for example, antibodies that bind to and antagonize RANK and an ICM, each chain bearing two variable domains (V_{H}A-V_{L}B and V_{H}B-V_{L}A or V_{L}A-V_{H}B and V_{L}B-V_{H}A). Typically, the polypeptide linkers joining the variable domains are short (i.e., from about 2, 3, 4, 5, 6, 7, 8, 9 or 10 amino acid residues). The short polypeptide linkers prevent the association of V_{H} and V_{L} domains on the same chain, and therefore promote the association of V_{H} and V_{L} domains on different chains. Heterodimers that form are functional against both target antigens, (e.g., V_{H}A-V_{L}B with V_{H}B-V_{L}A or V_{L}A-V_{H}B with V_{L}B-V_{H}A). However, homodimers can also form (e.g., V_{H}A-V_{L}B with V_{H}A-V_{L}B, V_{H}B-V_{L}A with V_{H}B-V_{L}A, etc.), leading to non-functional molecules. Several strategies are known in the art for preventing homodimerization, including the introduction of disulphide bonds to covalently join the two polypeptide chains, modification of the polypeptide chains to include large amino acids on one chain and small amino acids on the other (knobs-into-holes structures, as discussed above and elsewhere herein), and addition of cysteine residues at C-terminal extensions. Another strategy is to join the two polypeptide chains by a polypeptide linker sequence, producing a single-chain diabody molecule (scDb) that exhibits a more compact structure than a taFv. ScDbs or diabodies can be also be fused to the IgG1 C_{H3} domain or the Fc region, producing di-diabodies. Examples of di-diabodies include, but are not limited to, scDb-Fc, db-Fc, scDb-C_{H3}, and db-C_{H3}. Additionally, scDbs can be used to make tetravalent bispecific molecules. By shortening the polypeptide linker sequence of scDbs from about 15 amino acids to about 5 amino acids, dimeric single-chain diabody molecules result, known as TandAbs (as described in Muller and Kontermann, in Bispecific Antibodies Kontermann R E (ed.), Springer Heidelberg Dordrecht London New York, 83-100 (2011)).

5.5 Other Conjugation Techniques for Antigen-Binding Molecule Generation

Another suitable strategy for generating multispecific antigen-binding molecules according to the present invention includes conjugating or otherwise linking heterodimerizing peptides to the C-terminus of the antibody molecules (e.g., scFvs or Fabs).

A non-limiting example of this strategy is the use of antibody fragments linked to jun-fos leucine zippers (e.g., scFv-Jun/Fos and Fab′-Jun/Fos).

An additional method for generating a bispecific antigen-binding molecules comprises derivatizing two antibodies with different antigen binding fragments with biotin and then linking the two antibodies via streptavidin, followed by purification and isolation of the resultant bispecific antibody.

Additional types of bispecific antigen-binding molecules according to the present invention include those that contain more than one antigen-binding site for each antigen. For example, additional V_{H} and V_{L} domains can be fused to the N-terminus of the V_{H} and V_{L} domains of an existing antibody, effectively arranging the antigen-binding sites in tandem. These types of antibodies are known as dual-variable-domain antibodies (DVD-Ig) (see, Tarcsa, E. et al., in Bispecific Antibodies. Kontermann, supra, pp. 171-185). Another method for producing antibodies that contain more than one antigen-binding site for an antigen is to fuse scFv fragments to the N-terminus of the heavy chain or the C-terminus of the light chain (discussed in more detail below).

The antibodies or antigen-binding fragments of a multispecific antigen-binding molecule complex or construct are independently selected from the group consisting of IgM, IgG, IgD, IgA, IgE, or fragments thereof, which are distinguished from each other by the amino acid sequence of the constant region of their heavy chains. Several of these Ig classes are further divided into subclasses, such as IgG1, IgG2, IgG3, and IgG4, IgA1 and IgA2. The heavy chain constant regions that correspond to the different classes of antibodies are called α, δ, ε, γ and μ, respectively. The light chain constant regions (C_{L}) which can be found in all five antibody classes are selected from κ (kappa) and λ (lambda). Antibody fragments that retain antigen recognition and binding capability that are Fab, Fab′, F(ab′)₂, and Fv fragments. Further, the first and second antigen binding fragments are connected either directly or by a linker (e.g., a polypeptide linker).

5.6 Generating Bispecific Antigen-Binding Molecules Using an IgG Scaffold.

Constant immunoglobulin domains can suitably be used to promote heterodimerization of two polypeptide chains (e.g., IgG-like antibodies). Non-limiting examples of this strategy for producing bispecific antibodies include the introduction of knobs-into-holes structures into the two polypeptides and utilization of the naturally occurring heterodimerization of the C_{L} and C_{H1} domains (see, Kontermann, supra, pp. 1-28 (2011) Ridgway et al., Protein Eng. 1996 July; 9(7):617-21; Atwell et al., J Mol Biol. 1997 Jul. 4; 270(1):26-35).

The majority of the recombinant antigen-binding molecules according to the present invention can be engineered to be IgG-like, meaning that they also include an Fc domain. Similar to diabodies that require heterodimerization of engineered polypeptide chains, IgG-like antigen-binding molecules also require heterodimerization to prevent the interaction of like heavy chains or heavy chains and light chains from two antibodies of different specificity (Jin, P. and Zhu, Z. In: Bispecific Antibodies. Kontermann RE (ed.), Springer Heidelberg Dordrecht London New York, pp. 151-169 (2011)).

Knobs-into-holes structures facilitate heterodimerization of polypeptide chains by introducing large amino acids (knobs) into one chain of a desired heterodimer and small amino acids (holes) into the other chain of the desired heterodimer. Steric interactions will favour the interaction of the knobs with holes, rather than knobs with knobs or holes with holes. In the context of bispecific IgG-like antibodies, like heavy chains can be prevented from homodimerizing by the introduction of knobs-into-holes (KiH) structures into the CH3 domain of the Fc region. Similarly, promoting the interaction of heavy chains and light chains specific to the same antigen can be accomplished by engineering KiH structures at the VH-VL interface. Specifically, in KiH methodology, large amino acid side chains are introduced into the CH3 domain of one of the heavy chains, which side chains fit into appropriately designed cavities in the CH3 domain of the other heavy chain (see, e.g., Ridgeway et al., Protein Eng. 9(1996), 617-621 and Atwell et al., J. Mol. Biol. 270(1997), 677-681, which are hereby incorporated by reference herein). Thus, heterodimers of the heavy chains tend to be more stable than either homodimer, and form a greater proportion of the expressed polypeptides. In addition, the association of the desired light-chain/heavy-chain pairings can be induced by modification of one Fab of the bispecific antibody (Fab region) to “swap” the constant or constant and variable regions between the light and heavy chains. Thus, in the modified Fab domain, the heavy chain would comprise, for example, CL-V_{H} or CL-V_{L} domains and the light chain would comprise CH_{I}-V_{L} or CH_{I}-V_{H} domains, respectively. This prevents interaction of the heavy/light chain Fab portions of the modified chains (i.e., modified light or heavy chain) with and the heavy/light chain Fab portions of the standard/non-modified arm. By way of explanation, the heavy chain in the Fab domain of the modified arm, comprising a CL domain, does not preferentially interact with the light chain of the non-modified arm/Fab domain, which also comprises a CL domain (preventing “improper” or undesired pairings of heavy/light chains). This technique for preventing association of “improper” light/heavy chains is termed “CrossMAb” technology and, when combined with KiH technology, results in remarkably enhanced expression of the desired bispecific molecules (see, e.g., Schaefer et al. Proc Natl Acad Sci USA. 2011; 108(27):11187-92; and U.S. Patent Publication No 2010/0159587, which are hereby incorporated by reference herein in their entirety). Other examples of KiH structures exist and the examples discussed above should not be construed to be limiting. Other methods to promote heterodimerization of Fc regions include engineering charge polarity into Fc domains (see, Gunasekaran et al., 2010) and SEED technology (SEED-IgG) (Davis et al., Protein Eng Des Sel. 2010 April; 23(4):195-202, 2010).

In specific embodiments, the multispecific antigen-binding molecules are CrossMAbs, which are derived from independent parental antibodies in which antibody domain exchange is based on KiH methodology. Light chain mispairing is overcome using domain crossovers and heavy chains heterodimerized using the KIH method. For the domain crossovers either the variable domains or the constant domains are swapped between light and heavy chains to create two asymmetrical Fab arms to avoid light-chain mispairing while the “crossover” keeps the antigen-binding affinity. In comparison with natural antibodies, CrossMAbs show higher stability. There are several different CrossMAb formats, such as Fab, V_{H}-V_{L} and C_{H1}-C_{L} exchanged in different regions. In preferred embodiments, the multispecific antigen-binding molecules are based on the CrossMAb^{CH1-CL} format, which exchanges the C_{H1} and C_{L} regions of the bispecific antibody.

Additional heterodimerized IgG-like antigen-binding molecules include, but are not limited to, heteroFc-scFvs, Fab-scFvs, IgG-scFv, and scFv-IgG. HeteroFc-scFvs link two distinct scFvs to heterodimerizable Fc domains while Fab-scFvs contain a Fab domain specific to one epitope linked to an scFv specific to a different epitope. IgG-scFv and scFv-IgG are Ig-like antibodies that have scFvs linked to their C-termini and N-termini, respectively (see, Kontermann R E (ed.), supra, pp. 151-169).

Representative CrossMAb embodiments encompass ones in which an engineered protuberance is created in the interface of a first IgG-like polypeptide by replacing at least one contact residue of that polypeptide within its C_{H3} domain. In particular, the contact residue to be replaced on the first polypeptide corresponds to an IgG residue at position 366 (residue numbering is according to Fc crystal structure (Deisenhofer, Biochem. 20:2361 [1981]) and wherein an engineered protuberance comprises replacing the nucleic acid encoding the original residue with nucleic acid encoding an import residue having a larger side chain volume than the original residue. Specifically, the threonine (T) residue at position 366 is mutated to tryptophan (W). In the second step, an engineered cavity is created in the interface of the second polypeptide by replacing at least one contact residue of the polypeptide within its C_{H}3 domain, wherein the engineered cavity comprises replacing the nucleic acid encoding an original residue with nucleic acid encoding an import residue having a smaller side chain volume than the original residue. Specifically, the contact residue to be replaced on the second polypeptide corresponds to an IgG residue at position 407. Specifically, the tyrosine (Y) residue at position 407 is mutated to alanine (A). This procedure can be engineered on different IgG subtypes, selected from the group consisting of IgG1, IgG2a, IgG2b, IgG3 and IgG4.

In another illustrative example of CrossMAb technology, the multispecific antigen-binding molecules can be based on the duobody platform/cFAE (GenMAb), as described for example in WO2008119353 and WO 2011131746 (each of which is hereby incorporated herein by reference in its entirety) in which the bispecific antibody is generated by separate expression of the component antibodies in two different host cells followed by purification and assembly into bi-specific heterodimeric antibodies through a controlled Fab-arm exchange between two monospecific antibodies. By introducing asymmetrical, matching mutations (e.g., F405L and K409R, according to EU numbering index) in the C_{H3} regions of two monospecific starting proteins, similar to the Fab-arm exchange can be forced to become directional, thereby yielding stable heterodimeric pairs under reducing conditions (as described, for example by Labrijn et al., Proc Natl Acad Sci USA 2013; 110(13):5145-5150; Gramer et al. MAbs 2013; 5(6): 962-973; Labrijn et al. Nature Protocols 2014; 9(10):2450-63, which are hereby incorporated by reference herein in their entirety). In practice, bispecific human IgG1 Abs can be produced from the two purified bivalent parental antibodies, each with the respective single complementary mutation: K409R or F405L. This same strategy can be performed on human IgG1, IgG2, IgG3 or IgG4 backbone (Labrijn 2013, supra).

Still other non-limiting examples of multi-specific antigen-binding molecules include multispecific, e.g., bispecific, antibody molecules that include a lambda chain polypeptide and a kappa light chain polypeptide (as described in WO 2018/057955), and are capable of binding two or more antigens. The basis for this approach is that by using by using one kappa light chain polypeptide and one lambda light chain polypeptide, mispairing of the light chains to the incorrect heavy chain is prevented in the context of a multispecific antibody molecule. In the design of a multispecific antibody molecule that include a lambda chain polypeptide and a kappa light chain polypeptide and which binds two antigens, including RANK and an anti-ICM antigen-binding molecule, four constructs are generated. Additional asymmetric changes to generate “Knobs-into-holes” structures in the two different C_{H3} domains, which facilitate heterodimerization of polypeptide chains by introducing large amino acids (knobs) into one chain of a desired heterodimer and small amino acids (holes) into the other chain of the desired heterodimer. The four fragments, when co-expressed in mammalian cells, assemble to form a multi-specific antibody molecule.

5.7 Electrostatic Steering

In other embodiments, the multispecific antigen-binding molecules are based on electrostatic steering, in which the charge complementarity at the C_{H3} domain is altered, through selected mutations, leading to enhanced antibody Fc heterodimer formation through electrostatic steering effects (Gunasekaran et al., 2010. J Biol Chem 285(25):19637-46; WO 2009089004 Al). This same strategy can be performed on human IgG1, IgG2, IgG3 or IgG4 backbone (WO 2009089004 Al).

5.8 Linkers.

Linkers may be used to covalently link different antigen-binding molecules to form a chimeric molecule comprising at least two antigen-binding molecules. The linkage between antigen-binding molecules may provide a spatial relationship to permit binding of individual antigen-binding molecules to their corresponding cognate epitopes. In this context, an individual linker serves to join two distinct functional antigen-binding molecules. Types of linkers include, but are not limited to, chemical linkers and polypeptide linkers.

The linker may be chemical and include for example an alkylene chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker can be biologically inactive, such as a PEG, polyglycolic acid, or polylactic acid chain, or can be biologically active, such as an oligo- or polypeptide that, when cleaved from the moieties, binds a receptor, deactivates an enzyme, etc. The linker may be attached to the first and second antibodies or antigen-binding fragments by any suitable bond or functional group, including carbon-carbon bonds, esters, ethers, amides, amines, carbonates, carbamates, sulfonamides, etc.

In certain embodiments, the linker represents at least one (e.g., 1, 2, 3, 4, 5, 6, 7, 8, 9, 10, or more) derivatized or non-derivatized amino acid. In illustrative examples of this type, the linker is preferably non-immunogenic and flexible, such as those comprising serine and glycine sequences or repeats of Ala-Ala-Ala. Depending on the particular construct, the linkers may be long (e.g., greater than 12 amino acids in length) or short (e.g., 1, 2, 3, 4, 5, 6, 7, 8, 9, 10, 11, 12 amino acids in length). For example, to make a single chain diabody, the first and the third linkers are preferably about 3 to about 12 amino acids in length (and more preferably about 5 amino acids in length), and the second linker is preferably longer than 12 amino acids in length (and more preferably about 15 amino acids in length). Reducing the linker length to below three residues can force single chain antibody fragments into the present invention allowing the bispecific antibody to become bivalent, trivalent, or tetravalent, as desired.

Representative peptide linkers may be selected from: [AAA]ₙ, [SGGGG]n, [GGGGS]ₙ, [GGGGG]ₙ, [GGGKGGGG]ₙ, [GGGNGGGG]ₙ, [GGGCGGGG]ₙ, wherein n is an integer from 1 to 10, suitably 1 to 5, more suitably 1 to 3.

### 6. Multispecific Antigen-Binding Constructs

One aspect of the present invention relates to chimeric constructs that comprise a plurality of antigen-binding molecules with different specificities that are fused to or otherwise conjugated together, either directly or via a linker. Illustrative constructs are provided below.

6.1 Anti-RANK-Anti-PD-1 Diabody

An alternative approach to developing multispecific antibodies is based on the single-chain diabody (scdiabody) format. Here, the variable domains from two antibodies, A and B, are expressed as a polypeptide chain, V_{HA}-V_{LB}-linker-V_{HB}-V_{LA}. The present invention contemplates multispecific constructs which are bispecific and comprise a RANK antagonist antigen-binding molecule and an anti-PD-1 antigen-binding molecule, representative examples of which comprise, consist or consist essentially of a sequence selected from the following:

[SEQ ID NO: 158]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSS[SGGGG]ₙeivltqspatlslspgera
tlscrasgsyssylawyqqkpgqaprlliydasnratgiparfsgsgsgt
dftltisslepedfavyycqqssnwprtfgqgtkveik[SGGGG]ₙQVQL
VESGGGVVQPGRSLRLDCKASGITFSNSGMHWVRQAPGKGLEWVAVIWYD
GSKRYYADSVKGRFTISRDNSKNTLFLQMNSLRAEDTAVYYCATNDDYWG
QGTLVTVSS[SGGGG]ₙsyeltqppsvsvspgqtasitcsgdklgdkyvc
wyqqkpggspvlviygdserpsgiperfsgsnsgntatltisgtravdea
dyycqawdsttplfgggtnltvl,

wherein:
Uppercase regular text corresponds to the variable heavy chain amino acid sequence of the anti-RANK MAb 3A3
Lowercase underlined text corresponds to the variable light chain amino acid sequence of the anti-PD-1 MAb nivolumab
Uppercase underlined text corresponds to the variable heavy chain amino acid sequence of anti-PD-1 MAb nivolumab,
Lowercase regular text corresponds to the variable light chain amino acid sequence of anti-RANK MAb 3A3,
Each occurrence of [SGGGG]ₙ is a flexible linker, wherein n=1, 2, 3, or 4, preferably n=1 for the first and third instances of the flexible linker, and n=3 for the second instance of the flexible linker.

6.2 Anti-RANK-Anti-PD-L1 Diabody

Alternatively, the bispecific constructs comprise an anti-RANK antigen-binding molecule and an anti-PD-L1 antigen-binding molecule, representative examples of which comprise, consist or consist essentially of a sequence selected from the following:

[SEQ ID NO: 159]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSS[SGGGG]ₙeivltqspgtlslspgera
tlscrasqrvsssylawyqqkpgqaprlliydasnratgipdrfsgsgsg
tdftltisrlepedfavyycqqygslpwtfgqgtkveik[SGGGG]ₙVQL
VESGGGLVQPGGSLRLSCAASGFTFSRYWMSWVRQAPGKGLEWVANIKQD
GSEKYYVDSVKGRFTISRDNAKNSLYLQMNSLRAEDTAVYYCAREGGWFG
ELAFDYWGQGTLVTVSS[SGGGG]ₙsyeltqppsvsvspgqtasitcsgd
klgdkyvcwyqqkpggspvlviygdserpsgiperfsgsnsgntatltis
gtravdeadyycqawdsttplfgggtnltvl,

wherein:
Uppercase regular text corresponds to the variable heavy chain amino acid sequence of the anti-RANK MAb 3A3
Lowercase underlined text corresponds to the variable light chain amino acid sequence of the anti-PD-L1 MAb durvalumab
Uppercase underlined text corresponds to the variable heavy chain amino acid sequence of anti-PD-L1 MAb durvalumab,
Lowercase regular text corresponds to the variable light chain amino acid sequence of anti-RANK MAb 3A3,
Each occurrence of [SGGGG]ₙ is a flexible linker, wherein n=1, 2, 3, or 4, preferably n=1 for the first and third instances of the flexible linker, and n=3 for the second instance of the flexible linker.

Alternatively, the bispecific constructs comprise an anti-RANK antigen-binding molecule and an anti-PD-L1 antigen-binding molecule, representative examples of which comprise, consist or consist essentially of a sequence selected from the following:

[SEQ ID NO: 160]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSS[SGGGG]ₙdiqmtqspsslsasvgdrv
titcrasqdvstavawyqqkpgkapklliysasflysgvpsrfsgsgsgt
dftltisslqpedfatyycqqylyhpatfgqgtkveik[SGGGG]ₙEVQL
VESGGGLVQPGGSLRLSCAASGFTFSDSWIHWVRQAPGKGLEWVAWISPY
GGSTYYADSVKGRFTISADTSKNTAYLQMNSLRAEDTAVYYCARRHWPGG
FDYWGQGTLVTVSS[SGGGG]ₙsyeltqppsvsvspgqtasitcsgdklg
dkyvcwyqqkpgqspvlviygdserpsgiperfsgsnsgntatltisgtr
avdeadyycgawdsttplfgggtnltvl,

wherein:
Uppercase regular text corresponds to the variable heavy chain amino acid sequence of the anti-RANK MAb 3A3
Lowercase underlined text corresponds to the variable light chain amino acid sequence of the anti-PD-L1 MAb atezolizumab,
Uppercase underlined text corresponds to the variable heavy chain amino acid sequence of anti-PD-L1 MAb atezolizumab,
Lowercase regular text corresponds to the variable light chain amino acid sequence of anti-RANK MAb 3A3,
Each occurrence of [SGGGG]ₙ is a flexible linker, wherein n=1, 2, 3, or 4, preferably n=1 for the first and third instances of the flexible linker, and n=3 for the second instance of the flexible linker.

6.3 Anti-RANKL-Anti-CTLA4 Diabody

Alternatively, the bispecific constructs comprise an anti-RANK antigen-binding molecule and an anti-CTLA4 antigen-binding molecule, representative examples of which comprise, consist or consist essentially a sequence selected from the following:

[SEQ ID NO: 161]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSS[SGGGG]ₙeivltqspgtlslspgera
tlscrasqsvgssylawyqqkpgqaprlliygafsratgipdrfsgsgsg
tdftltisrlepedfavyycqqygsspwtfgqgtkveik[SGGGG]ₙQVQ
LVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLG
PFDYWGQGTLVTVSS[SGGGG]ₙsyeltqppsysyspgqtasitcsgdki
gdkyvcwyqqkpgqspvlviygdserpsgiperfsgsnsgntatltisgt
ravdeadyyccqawdsttplfgggtnltvl,

wherein:
Uppercase regular text corresponds to the variable heavy chain amino acid sequence of the anti-RANK MAb 3A3
Lowercase underlined text corresponds to the variable light chain amino acid sequence of the anti-CTLA4 MAb ipilimumab,
Uppercase underlined text corresponds to the variable heavy chain amino acid sequence of anti-CTLA4 MAb ipilimumab,
Lowercase regular text corresponds to the variable light chain amino acid sequence of anti-RANK MAb 3A3,
Each occurrence of [SGGGG]ₙ is a flexible linker, wherein n=1, 2, 3, or 4, preferably n=1 for the first and third instances of the flexible linker, and n=3 for the second instance of the flexible linker.

Alternatively, the bispecific constructs comprise an anti-RANK antigen-binding molecule and an anti-CTLA4 antigen-binding molecule, representative examples of which comprise, consist or consist essentially a sequence selected from the following:

[SEQ ID NO: 162]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSS[SGGGG]ₙdigmtqspssIsasvgdrv
titcrasgsinsyldwyqqkpgkapklliyaasslgsgvpsrfsgsgsgt
dftltisslqpedfatyycqqyystpftfgpgtkveik[SGGGG]ₙQVQL
VESGGGVVQPGRSLRLSCAASGFTFSSYTMHWVRQAPGKGLEWVTFISYD
GNNKYYADSVKGRFTISRDNSKNTLYLQMNSLRAEDTAIYYCARTGWLGP
FDYWGQGTLVTVSS[SGGGG]ₙsyeltqppsvsvspgqtasitcsgdkig
dkyvcwyqqkpgqspvlviygdserpsgiperfsgsnsgntatltisgtr
avdeadyycqawdsttplfgggtnltvl,

wherein:
Uppercase regular text corresponds to the variable heavy chain amino acid sequence of the anti-RANK MAb 3A3
Lowercase underlined text corresponds to the variable light chain amino acid sequence of the anti-CTLA4 MAb tremelimumab,
Uppercase underlined text corresponds to the variable heavy chain amino acid sequence of anti-CTLA4 MAb tremelimumab,
Lowercase regular text corresponds to the variable light chain amino acid sequence of anti-RANK MAb 3A3,
Each occurrence of [SGGGG]ₙ is a flexible linker, wherein n=1, 2, 3, or 4, preferably n=1 for the first and third instances of the flexible linker, and n=3 for the second instance of the flexible linker.

6.4 Anti-RANK—Anti-PD-L1 CrossMAb Constructs

The present invention also contemplates CrossMAb multispecific antigen-binding molecules. In a first step of CrossMAb construction, an engineered protuberance is created in the interface of a first IgG-like polypeptide by replacing at least one contact residue of that polypeptide within its C_{H3} domain. Specifically, the contact residue to be replaced on the first polypeptide corresponds to an IgG residue at position 366 (residue numbering is according to Fc crystal structure (Deisenhofer, Biochem. 20:2361 [1981]) and wherein an engineered protuberance comprises replacing the nucleic acid encoding the original residue with nucleic acid encoding an import residue having a larger side chain volume than the original residue. Specifically, the threonine (T) residue at position 366 is mutated to tryptophan (W). In the second step, an engineered cavity is created in the interface of the second polypeptide by replacing at least one contact residue of the polypeptide within its C_{H}3 domain, wherein the engineered cavity comprises replacing the nucleic acid encoding an original residue with nucleic acid encoding an import residue having a smaller side chain volume than the original residue. Specifically, the contact residue to be replaced on the second polypeptide corresponds to an IgG residue at position 407. Specifically, the tyrosine (Y) residue at position 407 is mutated to alanine (A). This procedure can be engineered on different IgG subtypes, selected from the group consisting of IgG1, IgG2a, IgG2b, IgG3 and IgG4.

In a subsequent step, to promote the discrimination between the two light chain/heavy chain interactions possible in a heterodimeric bi-specific IgG, the association of the desired light-chain/heavy-chain pairings can be induced by modification of one Fab of the bispecific antibody (Fab region) to “swap” the constant or constant and variable regions between the light and heavy chains (see, e.g., Schaefer et al., 2011, supra). Thus, in the modified Fab domain, the heavy chain would comprise, for example, C_{L}-V_{H} or C_{L}-V_{L} domains and the light chain would comprise C_{H1}-V_{L} or C_{H1}-V_{H} domains, respectively. This prevents interaction of the heavy/light chain Fab portions of the modified chains (i.e., modified light or heavy chain) with and the heavy/light chain Fab portions of the standard/non-modified arm. By way of explanation, the heavy chain in the Fab domain of the modified arm, comprising a C_{L} domain, does not preferentially interact with the light chain of the non-modified arm/Fab domain, which also comprises a CL domain (preventing “improper” or undesired pairings of heavy/light chains). This technique for preventing association of “improper” light/heavy chains is termed “CrossMAb” technology and, when combined with KiH technology, results in remarkably enhanced expression of the desired bispecific molecules (see, e.g., Schaefer et al., 2011, supra).

Production of the heterodimeric bi-specific IgG antibodies is achieved by first cloning each of the antibody genes encoding the 4 chains of the bi-specific IgG into mammalian expression vectors to enable secretory expression in mammalian cells (such as HEK293). Each of the antibody chain cDNAs is transfected together at equimolar ratios into HEK293 cells using 293fectin or similar techniques and antibody containing cell culture supernatants are harvested and antibodies are purified from supernatants using protein A Sepharose.

In some embodiments, a bi-specific heterodimeric IgG composed of both an anti-RANK antigen-binding molecule and an anti-PD-L1 antigen-binding molecule can be constructed using 2 heavy and 2 light chain constructs, in which one of the heavy chain C_{H3} domain is altered at position 366 (T366W), termed the “knob” and the other heavy chain C_{H3} domain is altered at position 407 (Y407A), termed the “hole” to promote KiH heterodimerization of the heavy chains.

In some embodiments, a bi-specific heterodimeric IgG composed of both an anti-RANK antigen-binding molecule and an anti-PD-L1 antigen-binding molecule can be constructed using 2 heavy and 2 light chain constructs, in which each of the heavy chain C_{H3} domain is altered at positions 234 (L234A), 235 (L235A), 329 (P329G) for reduced FcγR and C1q interactions.

6.4.1 Constructs for Multispecific CrossMAb Using C_{H1}-C_{L} Interchange which Binds Both RANK and PD-L1

An illustrative multispecific CrossMAb molecule may comprise heavy and light chain sequences derived from the anti-RANK 3A3 antibody and atezolizumab IgG1 and the desired light-chain/heavy-chain pairings can be induced by modification of the Fab domain of the anti-RANK antigen-binding molecule, such that the C_{H1} and C_{L} domains are interchanged between Ig chains.

For the purposes of this construction, the anti-RANK 3A3 V_{H}1 domain is fused in tandem with a human IgG1 C_{H1} domain derived from atezolizumab (or another suitable human IgG1) and has the following AA sequence:

[SEQ ID NO: 163]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSSastkgpsvfplapsskstsggtaalgc
lvkdyfpepvtvswnsgaltsgvhtfpavlqssglyslssvvtvpssslg
tqtyicnvnhkpsntkvdkkv,

wherein:
Anti-RANK 3A3 V_{H} is in regular uppercase text; and
Atezolizumab CH domain is in bold lowercase text.

For the purposes of this construction, the anti-RANK 3A3 V_{L} domain is fused in tandem with a human lambda C_{L} domain derived from lambda-1 light chain Uniprot sequence PODOX8 (or another suitable human lambda or kappa C_{L} sequence) and has the following AA sequence:

[SEQ ID NO: 164]
SYELTQPPSVSVSPGQTASITCSGDKLGDKYVCWYQQKPGQSPVLVIYGD
SERPSGIPERFSGSNSGNTATLTISGTRAVDEADYYCQAWDSTTPLFGGG
TNLTVLgqpkanptvtlfppsseelqankatlyclisdfypgavtvawka
dgspvkagvettkpskqsnnkyaassylsltpeqwkshrsyscqvthegs
tvektvaptecs,

wherein:
Anti-RANK 3A3 V_{L} is in regular uppercase text; and
PODOX8 C_{L} domain is in bold lowercase text.

In order to generate a multispecific CrossMAb using C_{H1}-C_{L} interchange which binds both RANK and PD-L1, the following four constructs are used for this construction.

Construct 1

Anti-RANK 3A3 CrossMAb CH1-CL huIgG1 KNOB Mutation, Heavy Chain

[SEQ ID NO: 165]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSSgqpkanptvtlfppsseelqankatly
clisdfypgavtvawkadgspvkagvettkpskqsnnkyaassylsltpe
qwkshrsyscqvthegstvektvaptecsepkscdkthtcppcpapeAAg
gpsvflfppkpkdtlmisrtpevtcvvvdvshedpevkfnwyvdgvevhn
aktkpreeqyastyrvvsvltvlhqdwlngkeykckvsnkalGapiekti
skakgqprepqvytlppsreenntknqvslWclvkgfypsdiavewesng
qpennykttppvldsdgsfflyskltvdksrwqqgnvfscsvmhealhnh
ytqkslslspgk,

wherein:
Anti-RANK 3A3 V_{H} [from SEQ ID NO:163] is in regular uppercase text;
C_{L} domain [from SEQ ID NO:164] is in bold lowercase text
Human IgG1 Hinge region is in underlined lowercase text;
Atezolizumab C_{H2}-C_{H3} domain is in regular lowercase text; and
T366W “knob” substitution and the L234A, L235A, P329G substitutions are in bold uppercase text.

Construct 2

Anti-RANK 3A3 CrossMAb C_{H1}-C_{L} Light Chain

[SEQ ID NO: 166]
SYELTQPPSVSVSPGQTASITCSGDKLGDKYVCWYQQKPGQSPVLVIYGD
SERPSGIPERFSGSNSGNTATLTISGTRAVDEADYYCQAWDSTTPLFGGG
TNLTVLastkgpsvfplapsskstsggtaalgclvkdyfpepvtvswnsg
altsgvhtfpavlcissglyslssvvtvpssslgtqtyicnvnhkpsntk
vdkkv,

wherein:
Anti-RANK 3A3 V_{L} is in regular uppercase text; and
C_{H1} domain [from SEQ ID NO:163] is in bold lowercase text.

Construct 3

### Atezolizumab IgG1 Hole Mutation, Heavy Chain

[SEQ ID NO: 167]
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSastkgpsvfplapsskstsggtaalgclykdy
fpepvtvswnsgaltsgvhtfpavlqssglyslssvvtvpssslgtqtyi
cnvnhkpsntkvdkkvepkscdkthtcppcpapeAAggpsvflfppkpkd
tlmisrtpevtcvvvdvshedpevkfnwyvdgvevhnaktkpreeqyast
yrvvsvltvlhqdwlngkeykckvsnkalGapiektiskakgqprepqvy
tlppsreemtknqvsltclvkgfypsdiavewesngqpennykttppvld
sdgsfflAskltvdksrwqqgnvfscsvmhealhnhytqkslslspgk,

wherein:
Atezolizumab VH is in regular uppercase text;
Atezolizumab CH1 domain is in bold lowercase text;
HuIgG1 Hinge region is in underlined lowercase text;
Atezolizumab HuIgG1 CH2-CH3 domain is in regular lowercase text; and
Y407A “hole” and the L234A, L235A, P329G substitutions are in bold uppercase text.

Construct 4

### Atezolizumab Light Chain

[SEQ ID NO: 168]
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,

The Cross-Mab approaches described herein can be used with other anti-PD-L1 and anti-PD-1 or anti-CTLA4 antigen-binding molecule sequences that substitute for the PD-L1 antigen-binding molecule sequences described above. Additionally, one use other IgG scaffolds, in particular IgG4.

In some embodiments, a bispecific heterodimeric IgG composed of both a RANK antagonist antigen-binding molecule and an anti-PD-1 antigen-binding molecule can be constructed using 2 heavy and 2 light chain constructs, in the context of a CrossMAb multispecific antigen-binding molecules in which one of the heavy chain C_{H3} domain is altered at position 366 (T366W), termed the “knob” and the other heavy chain C_{H3} domain is altered at position 407 (Y407A), termed the “hole” to promote KiH heterodimerization of the heavy chains.

6.4.2 Constructs for Multispecific CrossMAb—C_{H1}-C_{L} Interchange—IgG4 which Binds Both RANK and PD-1

An illustrative multispecific CrossMAb molecule may comprise heavy and light chain sequences derived from the anti-RANK 3A3 antibody and nivolumab IgG4 and the desired light-chain/heavy-chain pairings can be induced by modification of the Fab domain of the anti-RANK antigen-binding molecule, such that the C_{H1} and C_{L} domains are interchanged between Ig chains. The following four constructs are used for this construction:

Construct 1

Anti-RANK 3A3 CrossMAb C_{H1}-C_{L} huIgG4 KNOB Mutation, Heavy Chain

[SEQ ID NO: 169]
EVQLVESGGGVVQPGTSLRLSCAASGFTFSSYAMHWVRQAPGKGLEWVAV
VSYDGSTKSYADSMKGRFTISRDNSKNTLYLQMNSLRAEDTAVYYCAKDP
ALRYFDWGYFQHWGQGTLVTVSSgqpkanptvtlfppsseelqankatly
clisdfypgavtvawkadgspvkagvettkpskqsnnkyaassylsItpe
qwkshrsyscqvthegstvektvaptecseskygppcpscpapeflggps
vflfppkpkdtlmisrtpevtcvvvdvsqedpevqfnwyvdgvevhnakt
kpreeqfnstyrvvsvltvlhqdwlngkeykckvsnkglpssiektiska
kgqprepqvytlppsqeemtknqvslWclvkgfypsdiavewesngqpen
nykttppvldsdgsfflysrltvdksrwqegnvfscsvmhealhnhytqk
slslslgk,

wherein:
Anti-RANK 3A3 V_{H} [from SEQ ID NO:163] is in regular uppercase text;
C_{L} domain [from SEQ ID NO:154] is in bold lowercase text;
IgG4 hinge region is in underlined lowercase text;
IgG4 C_{H2}-C_{H3} domain is in regular lowercase text; and
T366W “knob” substitution is in bold uppercase text.

Construct 2

Anti-RANK 3A3 CrossMAb C_{H1}-C_{L} Light Chain

[SEQ ID NO: 166]
SYELTQPPSVSVSPGQTASITCSGDKLGDKYVCWYQQKPGQSPVLVIYGD
SERPSGIPERFSGSNSGNTATLTISGTRAVDEADYYCQAWDSTTPLFGGG
TNLTVLastkgpsvfplapsskstsggtaalgclvkdyfpepvtvswnsg
altsgvhtfpavlqssglyslssvvtvpssslgtqtyicnvnhkpsntkv
dkkv

wherein:
Anti-RANK 3A3 V_{L} is in regular uppercase text; and
C_{H1} domain [from SEQ ID NO:AAA] is in bold lowercase text.

Construct 3

### Nivolumab IgG₄ Hole Mutation, Heavy Chain

[SEQ ID NO: 170]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSastkgpsvfplapcsrstsestaalgclvkdyfpepv
tvswnsgaltsgvhtfpavlqssglyslssvvtvpssslgtktytcnvdh
kpsntkvdkrveskygppcppcpapeflggpsvflfppkpkdtlmisrtp
evtcvvvdvsqedpevqfnwyvdgvevhnaktkpreeqfnstyrvvsvlt
ylhqdwlngkeykckvsnkglpssiektiskakgqprepqvytlppsqee
nntknqvsltclvkgfypsdiavewesngqpennykttppvldsdgsffl
Asrltvdksrwqegnvfscsvmhealhnhytqkslslslgk,

wherein:
Nivolumab V_{H} is in regular uppercase text;
Nivolumab C_{H1} domain is in bold lowercase text;
IgG₄ hinge region is in underlined lowercase text;
IgG₄ C_{H2}-C_{H3} domain is in regular lowercase text; and
Y407A “hole” substitution is in bold uppercase text.

Construct 4

### Nivolumab Light Chain

[SEQ ID NO: 171]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

For human IgG4, engineering mutations S228P/L235E variant (SPLE) has previously demonstrated minimal FcγR binding (Newman et al., 2001, Clin. Immunol. 98, 164-174). Mutations in IgG1 or IgG4 Fc domains can be combined, for instance combining the LALA mutations in human IgG1 with a mutation at P329G or combining the SPLE mutation in human IgG4 with a mutation at P329G, will completely abolished FcγR and C1q interactions (Schlothauer et al., 2016, Protein Eng Des. Sel. 29, 457-466).

In some embodiments, a bispecific heterodimeric IgG4 composed of both an anti-RANK antigen-binding molecule and an anti-PD-1 antigen-binding molecule can be constructed using 2 heavy and 2 light chain constructs, in which each of the heavy chain C_{H3} domain is altered at positions 228 (S228P), 235 (L235E), 329 (P329G) for reduced FcγR and C1q interactions.

### 7. Pharmaceutical Compositions

The pharmaceutical compositions of the present invention generally comprise a RANK antagonist antigen-binding molecule or a therapeutic combination as described herein, formulated with one or more pharmaceutically-acceptable carriers. Optionally, the pharmaceutical composition comprises one or more other compounds, drugs, ingredients and/or materials. Regardless of the route of administration selected, the RANK antagonist antigen-binding molecules or therapeutic combinations of the present invention are formulated into pharmaceutically-acceptable dosage forms by conventional methods known to those of skill in the art (see, e.g., Remington, The Science and Practice of Pharmacy (21^{st} Edition, Lippincott Williams and Wilkins, Philadelphia, Pa.)).

The pharmaceutically acceptable carrier includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the RANK antagonist antigen-binding molecule or therapeutic combination is administered by intravenous infusion or injection. In another preferred embodiment, the RANK antagonist antigen-binding molecule or therapeutic combination is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically should be sterile and stable under the conditions of manufacture and storage. The composition can be formulated as a solution, microemulsion, dispersion, liposome, or other ordered structure suitable to high antigen-binding molecule concentration. Sterile injectable solutions can be prepared by incorporating the active compound (i.e., RANK antagonist antigen-binding molecule or therapeutic combination) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In specific embodiments, a RANK antagonist antigen-binding molecule or a therapeutic combination as described herein may be conjugated to a vehicle for cellular delivery. In these embodiments, typically an antibody of the disclosure, which may or may not be conjugated to a detectable label and/or ancillary therapeutic agent, is encapsulated in a suitable vehicle to either aid in the delivery of the antigen-binding molecule or a therapeutic combination to target cells, to increase the stability of the antigen-binding molecule or a therapeutic combination, or to minimize potential toxicity of the antigen-binding molecule or a therapeutic combination. As will be appreciated by a skilled artisan, a variety of vehicles are suitable for delivering an antibody of the present disclosure. Non-limiting examples of suitable structured fluid delivery systems may include nanoparticles, liposomes, microemulsions, micelles, dendrimers and other phospholipid-containing systems. Methods of incorporating antibodies into delivery vehicles are known in the art. Although various embodiments are presented below, it will be appreciate that other methods known in the art to incorporate an antigen-binding molecule or a therapeutic combination of the disclosure into a delivery vehicle are contemplated.

In some embodiments, a liposome delivery vehicle may be utilized. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the antigen-binding molecule or a therapeutic combination of the invention may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the antibody of the disclosure (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In other embodiments, an antigen-binding molecule or a therapeutic combination of the disclosure may be delivered to a cell as a microemulsion. Microemulsions are generally clear, thermodynamically stable solutions comprising an aqueous solution, a surfactant, and “oil”. The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The antibody of the disclosure may be encapsulated in a microemulsion by any method generally known in the art.

In yet other embodiments, an antigen-binding molecule or a therapeutic combination of the present invention may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate antibodies of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

A RANK antagonist antigen-binding molecule or therapeutic combination of the disclosure can be administered by a variety of methods known in the art, although for many therapeutic applications, the preferred route/mode of administration is intravenous injection or infusion. In one embodiment, the RANK antagonist antigen-binding molecule or therapeutic combination is administered by intravenous infusion at a rate of more than 20 mg/min, e.g., 20-40 mg/min, and preferably greater than or equal to 40 mg/min to reach a dose of about 35 to 440 mg/m², preferably about 70 to 310 mg/m², and more preferably, about 110 to 130 mg/m². In another embodiment, the RANK antagonist antigen-binding molecule or therapeutic combination is administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the RANK antagonist antigen-binding molecule or therapeutic combination can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Pharmaceutical compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for an effective amount of an RANK antagonist antigen-binding molecule or therapeutic combination is 0.1-30 mg/kg, more preferably 1-25 mg/kg. Dosages and therapeutic regimens of the RANK antagonist antigen-binding molecule or therapeutic combination can be determined by a skilled artisan. In certain embodiments, the RANK antagonist antigen-binding molecule or therapeutic combination is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the RANK antagonist antigen-binding molecule or therapeutic combination is administered at a dose from about 10 to 20 mg/kg every other week.

The RANK antagonist antigen-binding molecule or therapeutic combination can be administered by intravenous infusion at a rate of more than 20 mg/min, e.g., 20-40 mg/min, and preferably greater than or equal to 40 mg/min to reach a dose of about 35 to 440 mg/m², preferably about 70 to 310 mg/m², and more preferably, about 110 to 130 mg/m². In embodiments, the infusion rate of about 110 to 130 mg/m² achieves a level of about 3 mg/kg. In one embodiment, the RANK antagonist antigen-binding molecule or therapeutic combination is administered (e.g., intravenously) at a dose from about 3 to 800 mg, e.g., about 3, 20, 80, 240, or 800 mg. In certain embodiments, the RANK antagonist antigen-binding molecule or therapeutic combination is administered alone at a dose from about 20 to 800 mg, e.g., about 3, 20, 80, 240, or 800 mg. In other embodiments, the RANK antagonist antigen-binding molecule or therapeutic combination is administered at a dose from about 3 to 240 mg, e.g., about 3, 20, 80, or 240 mg, in combination with a second agent or therapeutic modality, e.g., an ancillary agent or therapeutic modality described herein. In one embodiment, the RANK antagonist antigen-binding molecule or therapeutic combination is administered every 2 weeks (e.g., during weeks 1, 3, 5, 7) during each 8 week cycle, e.g., up to 96 weeks.

The RANK antagonist antigen-binding molecule or therapeutic combination can be administered by intravenous infusion at a rate of more than 20 mg/min, e.g., 20-40 mg/min, and preferably greater than or equal to 40 mg/min to reach a dose of about 35 to 440 mg/m², preferably about 70 to 310 mg/m², and more preferably, about 110 to 130 mg/m². In embodiments, the infusion rate of about 110 to 130 mg/m² achieves a level of about 3 mg/kg. In other embodiments, the RANK antagonist antigen-binding molecule or therapeutic combination is administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², and more preferably, about 10 mg/m². In some embodiments, the RANK antagonist antigen-binding molecule or therapeutic combination is infused over a period of about 30 min.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include an effective amount of RANK antagonist antigen-binding molecule or therapeutic combination. The effective amount may be a “therapeutically effective amount” or a “prophylactically effective amount” of a RANK antagonist antigen-binding molecule or therapeutic combination of the invention. A “therapeutically effective amount” refers to an amount effective, at dosages and for periods of time necessary, to achieve the desired therapeutic result. A therapeutically effective amount of the RANK antagonist antigen-binding molecule or therapeutic combination may vary according to factors such as the disease state, age, sex, and weight of the individual, and the ability of the RANK antagonist antigen-binding molecule or therapeutic combination to elicit a desired response in the individual. A therapeutically effective amount is also one in which any toxic or detrimental effects of the RANK antagonist antigen-binding molecule or therapeutic combination is outweighed by the therapeutically beneficial effects. A “therapeutically effective dosage” preferably inhibits a measurable parameter, e.g., osteoclast proliferation or tumor growth rate by at least about 20%, more preferably by at least about 40%, even more preferably by at least about 60%, and still more preferably by at least about 80% relative to untreated subjects. The ability of a compound to inhibit a measurable parameter, e.g., an osteopenic disorder, myopathy or cancer, can be evaluated in an animal model system predictive of efficacy in human osteopenic disorders, myopathies or cancers. Alternatively, this property of a composition can be evaluated by examining the ability of the compound to inhibit, for example in in vitro by assays known to the skilled practitioner.

By contrast, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### 8. Ancillary Treatments

The RANK antagonist antigen-binding molecules, therapeutic combinations and pharmaceutical compositions disclosed herein may be co-administered with one or more additional therapeutic agents (e.g., bone resorptive agents, anti-cancer agents, cytotoxic or cytostatic agents, hormone treatment, vaccines, and/or other immunotherapies). Alternatively or in addition, the RANK antagonist antigen-binding molecules, therapeutic combinations and pharmaceutical compositions are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications.

Combination therapies contemplated for use with the RANK antagonist antigen-binding molecules of the invention include bone anti-resorptive agents such as but not limited to: bone morphogenic factors designated BMP-1 to BMP-12; transforming growth factor-β and TGF-β family members; fibroblast growth factors FGF-1 to FGF-10; interleukin-1 inhibitors (including IL-1ra, antibodies to IL-1 and antibodies to IL-1 receptors); TNFα inhibitors (including etanercept, adalimumab and infliximab); RANK ligand inhibitors (including soluble RANK, osteoprotegerin and antagonistic antibodies that specifically bind RANK ligand), Dkk-1 inhibitors (e.g., anti-Dkk-1 antibodies) parathyroid hormone, E series prostaglandins, bisphosphonates and bone-enhancing minerals such as fluoride and calcium. Anabolic agents that can be used in combination with the RANK antagonist antigen-binding molecules include parathyroid hormone and insulin-like growth factor (IGF), wherein the latter agent is preferably complexed with an IGF binding protein. An IL-1 receptor antagonist suitable for such combination treatment is described in WO89/11540 and a suitable soluble TNF receptor-1 is described in WO98/01555. Exemplary RANK ligand antagonists are disclosed, for example, in WO 03/086289, WO 03/002713, U.S. Pat. Nos. 6,740,511 and 6,479,635. Alternative combination therapies encompassed for use with the RANK antagonist antigen-binding molecules of the invention include myopathy treatment agents, illustrative examples of which include nifuroxazide, ketoprofen, sulfasalazine, 5,15-diphenylporphyrin, pargyline hydrochloride, metolazone, zimelidine dihydrochloride monohydrate, miconazole, ticlopidine hydrochloride, iohexol, benoxinate hydrochloride, nimodipine, tranylcypromine hydrochloride, and AG490.

In other examples, the therapeutic combination disclosed herein can be combined with a standard cancer treatment, including any one or more antibody molecules, chemotherapy, other anti-cancer therapy (e.g., targeted anti-cancer therapies, or oncolytic drugs), cytotoxic agents, immune-based therapies (e.g., cytokines), surgical and/or radiation procedures. Exemplary cytotoxic agents that can be administered in combination with include antimicrotubule agents, topoisomerase inhibitors, anti-metabolites, mitotic inhibitors, alkylating agents, anthracyclines, Vinca alkaloids, intercalating agents, agents capable of interfering with a signal transduction pathway, agents that promote apoptosis, proteasome inhibitors, and radiation (e.g., local or whole body irradiation).

In some embodiments, the therapeutic combination is used in combination with a chemotherapeutic agent that is already routinely used as standard in the treatment of the subject. Suitable chemotherapeutic agents include, but are not limited to, anastrozole (ARIMIDEX), bicalutamide (CASODEX), bleomycin sulfate (BLENOXANE), busulfan (MYLERAN), busulfan injection (BUSULFEX), capecitabine (XELODA), N4-pentoxycarbonyl-5-deoxy-5-fluorocytidine, carboplatin (PARAPLATIN), carmustine (BICNU), chlorambucil (LEUKERAN), cisplatin (PLATINOL), cladribine (LEUSTATIN), cyclophosphamide (CYTOXAN or NEOSAR), cytarabine, cytosine arabinoside (CYTOSAR-U), cytarabine liposome injection (DEPOCYT), dacarbazine (DTIC-DOME), dactinomycin (actinomycin D, Cosmegan), daunorubicin hydrochloride (CERUBIDINE), daunorubicin citrate liposome injection (DAUNOXOME), dexamethasone, docetaxel (TAXOTERE), doxorubicin hydrochloride (ADRIAMYCIN, RUBEX), etoposide (VEPESID), fludarabine phosphate (FLUDARA), 5-fluorouracil (ADRUCIL, EFUDEX), flutamide (EULEXIN), tezacitibine, gemcitabine (GEMZAR), hydroxyurea (HYDREA), idarubicin (IDAMYCIN), ifosfamide (IFEX), irinotecan (CAMPTOSAR), L-asparaginase (ELSPAR), leucovorin calcium, melphalan (ALKERAN), 6-mercaptopurine (PURINETHOL), methotrexate (FOLEX), mitoxantrone (NOVANTRONE), mylotarg, paclitaxel (TAXOL), nab-paclitaxel (ABRAXANE), phoenix (Yttrium90/MX-DTPA), pentostatin, polifeprosan 20 with carmustine implant (GLIADEL wafer), tamoxifen citrate (NOLVADEX), teniposide (VUMON), 6-thioguanine, thiotepa, tirapazamine (TIRAZONE), topotecan hydrochloride for injection (HYCAMPTIN), vinblastine (VELBAN), vincristine (ONCOVIN), and vinorelbine (NAVELBINE).

Exemplary alkylating agents include nitrogen mustards, ethylenimine derivatives, alkyl sulfonates, nitrosoureas and triazenes): uracil mustard (AMINOURACIL MUSTARD, CHLORETHAMINACIL, DEMETHYLDOPAN, DESMETHYLDOPAN, HAEMANTHAMINE, NORDOPAN, URACIL NITROGEN MUSTARD, URACILLOST, URACILMOSTAZA, URAMUSTIN, URAMUSTINE), chlormethine (MUSTARGEN), cyclophosphamide (CYTOXAN, NEOSAR, CLAFEN, ENDOXAN, PROCYTOX, REVIMMUNE), dacarbazine (DTIC-DOME), ifosfamide (MITOXANA), melphalan (ALKERAN), chlorambucil (LEUKERAN), pipobroman (AMEDEL, VERCYTE), triethylenemelamine (HEMEL, HEXALEN, HEXASTAT), triethylenethiophosphoramine, Temozolomide (TEMODAR and TEMODAL), thiotepa (THIOPLEX), busulfan (BUSILVEX, MYLERAN), carmustine (BICNU), lomustine (CCNUCEENU), streptozocin (ZANOSAR), oxaliplatin (ELOXATIN); dactinomycin (also known as actinomycin-D, COSMEGEN); melphalan (L-PAM, L-sarcolysin, phenylalanine mustard, ALKERAN), altretamine (hexamethylmelamine (HMM), HEXALEN), bendamustine (TREANDA), busulfan (BUSULFEX and MYLERAN), carboplatin (PARAPLATIN), cisplatin (CDDP, PLATINOL and PLATINOL-AQ), chlorambucil (LEUKERAN), dacarbazine (DTIC, DIC and imidazole carboxamide, DTIC-DOME), altretamine (hexamethylmelamine (HMM), HEXALEN), ifosfamide (IFEX), prednumustine, procarbazine (MATULANE), and thiotepa (thiophosphoamide, TESPA and TSPA, THIOPLEX).

Exemplary anthracyclines include, e.g., doxorubicin (ADRIAMYCIN and RUBEX), bleomycin (LENOXANE), daunorubicin (dauorubicin hydrochloride, daunomycin, rubidomycin hydrochloride, and CERUBIDINE), daunorubicin liposomal (daunorubicin citrate liposome, and DAUNOXOME), mitoxantrone (DHAD and NOVANTRONE), epirubicin (ELLENCE), idarubicin (IDAMYCIN and IDAMYCIN PFS), mitomycin C (MUTAMYCIN), geldanamycin, herbimycin, ravidomycin, and desacetylravidomycin.

Exemplary vinca alkaloids that can be used in combination with the agents, antibodies and methods discloses above and elsewhere herein include, but are not limited to, vinorelbine tartrate (NAVELBINE), vincristine (ONCOVIN), vindesine (ELDISINE), and vinblastine (vinblastine sulfate, vincaleukoblastine, VLB, ALKABAN-AQ and VELBAN).

Exemplary proteasome inhibitors that can be used with the present invention include, but are not limited to, bortezomib (VELCADE), carfilzomib (PX-171-007), marizomib (NPI-0052), ixazomib citrate (MLN-9708), delanzomib (CEP-18770), O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912); danoprevir (RG7227, CAS 850876-88-9), ixazomib (MLN2238, CAS 1072833-77-2), and (S)-N-[(phenylmethoxy)carbonyl]-L-leucyl-N-(1-formyl-3-methylbutyl)-L-Leucinamide (MG-132, CAS 133407-82-6).

In some embodiments, the therapeutic combinations may be used in combination with a tyrosine kinase inhibitor (e.g., a receptor tyrosine kinase (RTK) inhibitor). Exemplary tyrosine kinase inhibitors include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor.

In some embodiments, the therapeutic combinations are used in combination with a hedgehog pathway inhibitor. Suitable hedgehog inhibitors known to be effective in the treatment of cancer include, but are not limited to, axitinib (AG013736), bosutinib (SKI-606), cediranib (RECENTIN, AZD2171), dasatinib (SPRYCEL, BMS-354825), erlotinib (TARCEVA), gefitinib (IRESSA), imatinib (GLEEVEC, CGP57148B, STI-571), lapatinib (TYKERB, TYVERB), lestaurtinib (CEP-701), neratinib (HKI-272), nilotinib (TASIGNA), semaxanib (semaxinib, SU5416), sunitinib (SUTENT, SU11248), toceranib (PALLADIA), vandetanib (ZACTIMA, ZD6474), vatalanib (PTK787, PTK/ZK), trastuzumab (HERCEPTIN), bevacizumab (AVASTIN), rituximab (RITUXAN), cetuximab (ERBITUX), panitumumab (VECTIBIX), ranibizumab (Lucentis), nilotinib (TASIGNA), sorafenib (NEXAVAR), alemtuzumab (CAMPATH), gemtuzumab ozogamicin (MYLOTARG), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, pazopanib hydrochloride, PD173074, Sorafenib Tosylate (Bay 43-9006), SU 5402, TSU-68 (SU6668), vatalanib, XL880 (GSK1363089, EXEL-2880), vismodegib (2-chloro-N-[4-chloro-3-(2-pyridinyl)phenyl]-4-(methylsulfonyl)-benzamide, GDC-0449 (as disclosed in PCT Publication No. WO 06/028958), 1-(4-Chloro-3-(trifluoromethyl)phenyl)-3-((3-(4-fluorophenyl)-3,4-dihydro-4-oxo-2-quinazolinyl)methyl)-urea (CAS 330796-24-2), N-[(2S,3R,3′R,3aS,4′aR,6S,6′aR,6′bS,7aR,12′aS,12′bS)-2′,3′,3a,4,4′,4′a,5,5′,6,6′,6′a,6′b,7,7′,7a,8′,10′,12′,12′a,12′b-Eicosahydro-3,6,11′,12′b-tetramethylspiro[furo[3,2-b]pyridine-2(3H),9′(1′H)-naphth[2,1-a]azulen]-3′-yl]-methanesulfonamide (IPI926, CAS 1037210-93-7), 4-Fluoro-N-methyl-N-[1-[4-(1-methyl-1H-pyrazol-5-yl)-1-phthalazinyl]-4-piperidinyl]-2-(trifluoromethyl)-benzamide (LY2940680, CAS 1258861-20-9), erismodegib (LDE225).

In certain embodiments, the therapeutic combinations are used in combination with a vascular endothelial growth factor (VEGF) receptor inhibitors, including but not limited to, bevacizumab (AVASTIN), axitinib (INLYTA), brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate), sorafenib (NEXAVAR), pazopanib (VOTRIENT), sunitinib malate (SUTENT), cediranib (AZD2171, CAS 288383-20-1), vargatef (BIBF1120, CAS 928326-83-4), foretinib (GSK1363089), telatinib (BAY57-9352, CAS 332012-40-5), apatinib (YN968D1, CAS 811803-05-1), imatinib (GLEEVEC), ponatinib (AP24534, CAS 943319-70-8), tivozanib (AV951, CAS 475108-18-0), regorafenib (BAY73-4506, CAS 755037-03-7), vatalanib dihydrochloride (PTK787, CAS 212141-51-0), brivanib (BMS-540215, CAS 649735-46-6), vandetanib (CAPRELSA or AZD6474), motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in the International PCT Publication No. WO 02/066470), dovitinib dilactic acid (TKI258, CAS 852433-84-2), linfanib (ABT869, CAS 796967-16-3), cabozantinib (XL184, CAS 849217-68-1), lestaurtinib (CAS 111358-88-4), N-[5-[[[5-(1,1-Dimethylethyl)-2-oxazolyl]methyl]thio]-2-thiazolyl]-4-piperidinecarboxamide (BMS38703, CAS 345627-80-7), (3R,4R)-4-Amino-1-((4-((3-methoxyphenyl)amino)pyrrolo[2,1-f][1,2,4]triazin-5-yl)methyl)piperidin-3-ol (BMS690514), N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8), 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0), and aflibercept (EYLEA).

In some embodiments, the therapeutic combinations are used in combination with a PI3K inhibitor. In one embodiment, the PI3K inhibitor is an inhibitor of delta and gamma isoforms of PI3K. Exemplary PI3K inhibitors that can be used in combination are described in, e.g., WO2010/036380, WO2010/006086, WO09/114870, WO05/113556, the contents of which are incorporated herein by reference. Suitably, PI3K inhibitors include 4-[2-(1H-Indazol-4-yl)-6-[[4-(methylsulfonyl)piperazin-1-yl]methyl]thieno[3,2-d]pyrimidin-4-yl]morpholine (also known as GDC-0941 (as described in International PCT Publication Nos. WO 09/036082 and WO 09/055730), 2-Methyl-2-[4-[3-methyl-2-oxo-8-(quinolin-3-yl)-2,3-dihydroimidazo[4,5-c]quinolin-1-yl]phenyl]propionitrile (BEZ235 or NVP-BEZ 235, as described in International PCT Publication No. WO06/122806); 4-(trifluoromethyl)-5-(2,6-dimorpholinopyrimidin-4-yl)pyridin-2-amine (BKM120 or NVP-BKM120, described in International PCT Publication No. WO2007/084786), tozasertib (VX680 or MK-0457, CAS 639089-54-6); (5Z)-5-[[4-(4-pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); 8-phenyl-2-(morpholin-4-yl)-chromen-4-one (LY294002, CAS 154447-36-6), 2-amino-8-ethyl-4-methyl-6-(1H-pyrazol-5-yl)pyrido[2,3-d]pyrimidin-7(8H)-one (SAR 245409 or XL 765), 1,3-dihydro-8-(6-methoxy-3-pyridinyl)-3-methyl-1-[4-(1-piperazinyl)-3-(trifluoromethyl)phenyl]-2H-imidazo[4,5-c]quinolin-2-one, (2Z)-2-butenedioate (1:1) (BGT 226), 5-fluoro-3-phenyl-2-[(1S)-1-(9H-purin-6-ylamino)ethyl]-4(3H)-quinazolinone (CAL101), 2-amino-N-[3-[N-[3-[(2-chloro-5-methoxyphenyl)amino]quinoxalin-2-yl]sulfamoyl]phenyl]-2-methylpropanamide (SAR 245408 or XL 147), and (S)-pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (BYL719).

In some embodiments, the therapeutic combinations are used in combination with a mTOR inhibitor, for example, one or more mTOR inhibitors chosen from one or more of rapamycin, temsirolimus (TORISEL), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587, ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and those described in PCT Publication No. WO03/064383), everolimus (ARINITOR or RAD001), rapamycin (AY22989, SIROLIMUS), simapimod (CAS 164301-51-3), emsirolimus, (5-{2,4-Bis[(3S)-3-methylmorpholin-4-yl]pyrido[2,3-d]pyrimidin-7-yl}-2-methoxyphenyl)methanol (AZD8055), 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4), and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1), (1r,4r)-4-(4-amino-5-(7-methoxy-1H-indol-2-yl)imidazo[1,5-f][1,2,4]triazin-7-yl)cyclohexanecarboxylic acid (OSI-027); and XL765.

In some embodiments, the therapeutic combinations are used in combination with a BRAF inhibitor, for example, GSK2118436, RG7204, PLX4032, GDC-0879, PLX4720, and sorafenib tosylate (Bay 43-9006). In further embodiments, a BRAF inhibitor includes, but is not limited to, regorafenib (BAY73-4506, CAS 755037-03-7), tuvizanib (AV951, CAS 475108-18-0), vemurafenib (ZELBORAF, PLX-4032, CAS 918504-65-1), encorafenib (also known as LGX818), 1-Methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8), 541-(2-Hydroxyethyl)-3-(pyridin-4-yl)-1H-pyrazol-4-yl]-2,3-dihydroinden-1-one oxime (GDC-0879, CAS 905281-76-7), 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885), (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662), and N-(3-(5-chloro-1H-pyrrolo[2,3-b]pyridine-3-carbonyl)-2,4-difluorophenyl)propane-1-sulfonamide (also known as PLX4720).

The therapeutic combinations can also be used in combination with a MEK inhibitor. Any MEK inhibitor can be used in combination including, but not limited to, selumetinib (5-[(4-bromo-2-chlorophenyl)amino]-4-fluoro-N-(2-hydroxyethoxy)-1-methyl-1H-benzimidazole-6-carboxamide (AZD6244 or ARRY 142886, described in PCT Publication No. WO2003/077914), trametinib dimethyl sulfoxide (GSK-1120212, CAS 1204531-25-80), RDEA436, N-[3,4-Difluoro-2-[(2-fluoro-4-iodophenyl)amino]-6-methoxyphenyl]-1-[(2R)-2,3-dihydroxypropyl]-cyclopropanesulfonamide (RDEA119 or BAY869766, described in PCT Publication No. WO2007/014011), AS703026, BIX 02188, BIX 02189, 2-[(2-Chloro-4-iodophenyl)amino]-N-(cyclopropylmethoxy)-3,4-difluoro-benzamide (also known as CI-1040 or PD184352, described in PCT Publication No. WO2000/035436), N-[(2R)-2,3-Dihydroxypropoxy]-3,4-difluoro-2-[(2-fluoro-4-iodophenyl)amino]-benzamide (PD0325901 and described in PCT Publication No. WO2002/006213), 2′-amino-3′-methoxyflavone (PD98059), 2,3-bis[amino[(2-aminophenyl)thio]methylene]-butanedinitrile (U0126 and described in U.S. Pat. No. 2,779,780), XL-518 (GDC-0973, Cas No. 1029872-29-4), G-38963, and G02443714 (also known as AS703206), or a pharmaceutically acceptable salt or solvate thereof. Other MEK inhibitors are disclosed in WO2013/019906, WO03/077914, WO2005/121142, WO2007/04415, WO2008/024725 and WO2009/085983, the contents of which are incorporated herein by reference. Further examples of MEK inhibitors include, but are not limited to, benimetinib (6-(4-bromo-2-fluorophenylamino)-7-fluoro-3-methyl-3H-benzoimidazole-5-carboxylic acid (2-hydroxyethyoxy)-amide (MEK162, CAS 1073666-70-2, described in PCT Publication No. WO2003/077914), 2,3-Bis[amino[(2-aminophenyl)thio]methylene]-butanedinitrile (U0126 and described in U.S. Pat. No. 2,779,780), (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (E6201, described in PCT Publication No. WO2003/076424), vemurafenib (PLX-4032, CAS 918504-65-1), (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5), pimasertib (AS-703026, CAS 1204531-26-9), 2-(2-Fluoro-4-iodophenylamino)-N-(2-hydroxyethoxy)-1,5-dimethyl-6-oxo-1,6-dihydropyridine-3-carboxamide (AZD 8330), and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, the therapeutic combinations are administered with a JAK2 inhibitor, for example, CEP-701, INCB18424, CP-690550 (tasocitinib). Exemplary JAK inhibitors include, but are not limited to, ruxolitinib (JAKAFI), tofacitinib (CP690550), axitinib (AG013736, CAS 319460-85-0), 5-Chloro-N2-[(1S)-1-(5-fluoro-2-pyrimidinyl)ethyl]-N4-(5-methyl-1H-pyrazol-3-y)-l2,4-pyrimidinediamine (AZD1480, CAS 935666-88-9), (9E)-15-[2-(1-Pyrrolidinyl)ethoxy]-7,12,26-trioxa-19,21,24-triazatetracyclo[18.3.1.12,5.114,18]-hexacosa-1(24),2,4,9,14,16,18(25),20,22-nonaene (SB-1578, CAS 937273-04-6), momelotinib (CYT 387), baricitinib (INCB-028050 or LY-3009104), pacritinib (SB1518), (16E)-14-Methyl-20-oxa-5,7,14,27-tetraazatetracyclo[19.3.1.12,6.18,12]heptacosa-1(25),2,4,6(27),8,10,12(26),16,21,23-decaene (SB 1317), gandotinib (LY 2784544), and N,N-cicyclopropyl-4-[(1,5-dimethyl-1H-pyrazol-3-yparnino]-6-ethyl-1,6-dihydro-1-methyl-imidazo[4,5-d]pyrrolo[2,3-b]pyridine-7-carboxamide (BMS 911543).

In yet other embodiments, the therapeutic combinations are administered in combination with an immunotherapy. Immunotherapy approaches, include for example cancer vaccines, an immunomodulator (e.g., an activator of a costimulatory molecule or an inhibitor of an inhibitory molecule), ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

The therapeutic combinations can be administered with one or more of the existing modalities for treating cancers, including, but not limited to: surgery; radiation therapy (e.g., external-beam therapy which involves three dimensional, conformal radiation therapy where the field of radiation is designed, local radiation (e.g., radiation directed to a preselected target or organ), or focused radiation). Focused radiation can be selected from the group consisting of stereotactic radiosurgery, fractionated stereotactic radiosurgery, and intensity-modulated radiation therapy. The focused radiation can have a radiation source selected from the group consisting of a particle beam (proton), cobalt-60 (photon), and a linear accelerator (x-ray), e.g., as described in WO2012/177624, which is incorporated herein by reference in its entirety.

Radiation therapy can be administered through one of several methods, or a combination of methods, including external-beam therapy, internal radiation therapy, implant radiation, stereotactic radiosurgery, systemic radiation therapy, radiotherapy and permanent or temporary interstitial brachytherapy. The term “brachytherapy,” refers to radiation therapy delivered by a spatially confined radioactive material inserted into the body at or near a tumor or other proliferative tissue disease site. The term is intended without limitation to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, I-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of I-125 or 1-131, or a radioactive fluid can be produced using a slurry of a suitable fluid containing small particles of solid radionuclides, such as Au-198, Y-90. Moreover, the radionuclide(s) can be embodied in gels or radioactive microspheres.

### 9. Therapeutic or Prophylactic Uses

The present invention also encompasses the use of the RANK antagonist antigen-binding molecules described herein, as well as therapeutic combinations based on those antigen-binding molecules for treating a range of conditions associated with RANK activation.

In particular, the RANK antagonist antigen-binding molecules described herein are contemplated for use in treating or inhibiting the development of conditions associated with activation of the RANKL/RANK signaling pathway. These conditions include, but are not limited to, osteopenic disorders, a myopathies and cancer.

In particular embodiments of these applications, the present invention provides methods for treating or inhibiting the development of bone loss in a subject in a subject, wherein the methods comprise administering to the subject an effective amount of a RANK antagonist antigen-binding molecule described herein, to thereby treat or inhibit the development of bone loss.

In other particular embodiments of these applications, the present invention provides methods for treating or inhibiting the development of a cancer associated with activation of the RANKL/RANK signaling pathway in a subject, wherein the methods comprise administering to the subject an effective amount of a RANK antagonist antigen-binding molecule described herein, thereby treating or inhibiting the development of the cancer. In specific embodiments, the cancer is selected from breast cancer including HR negative (e.g., ER−; PR−; HER2−; ER−, PR−; ER−, HER2−; PR−, HER2−; and ER−, PR−, HER2−) breast cancer, BRCA-1 mutation positive breast cancer, HR negative (e.g., ER−; PR−; HER2−; ER−, PR−; ER−, HER2−; PR−, HER2−; and ER−, PR−, HER2−) and BRCA-1 mutation positive breast cancer, prostate cancer, NSCLC including KRAS mutant or KRAS and LKB1 mutant NSCLC, and RCC cells including ccRCC.

Additionally, the therapeutic combinations of the present invention which employ the RANK antagonist antigen-binding molecules described herein in combination with one or more anti-ICM antigen-binding molecules or with one or more anti-AMA antigen-binding molecules have particular utility for stimulating or augmenting immunity, for inhibiting the development or progression of immunosuppression or tolerance to a tumor, or for inhibiting the development, progression or recurrence of cancer.

In accordance with the present invention, it is proposed that the agents of the present invention (e.g., RANK antagonist antigen-binding molecules and therapeutic combinations) may be used therapeutically after a condition (e.g., osteopenic disorder, myopathy or cancer) is diagnosed, or may be used prophylactically before the subject develops a condition (e.g., osteopenic disorder, myopathy or cancer). The present invention therefore provides a RANK antagonist antigen-binding molecule for use in (a) treating a condition associated with activation of the RANKL/RANK signaling pathway, (b) delaying onset of a condition associated with activation of the RANKL/RANK signaling pathway, (c) delaying progression of a condition associated with activation of the RANKL/RANK signaling pathway, and (d) prolonging the survival of a patient suffering from a condition associated with activation of the RANKL/RANK signaling pathway. Osteopenic disorders encompassed by the present invention include, but are not limited to, osteoporosis, periodontitis, cancer associated bone metastasis, multiple myeloma, rheumatoid arthritis, psoriatic arthritis, familial expansile osteolysis, Paget's disease (including juvenile Paget's disease) osteoclastoma, bone loss associated with chronic viral infection and adult and child leukemias, and periprosthetic bone loss, as well as cancers in which osteoclast activity is increased and bone resorption is induced, such as breast, prostate, and multiple myeloma. Representative myopathies include inherited myopathies such as dystrophies, myotonias, congenital myopathies (e.g., nemaline myopathy, multi/minicore myopathy, and centronuclear myopathy), mitochondrial myopathies, familial periodic myopathies, inflammatory myopathies and metabolic myopathies (e.g., glycogen storage diseases and lipid storage disorder), as well as acquired myopathies such as external substance induced myopathy (e.g., drug-induced myopathy and glucocorticoid myopathy, alcoholic myopathy, and myopathy due to other toxic agents), myositis (e.g., dermatomyositis, polymyositis and inclusion body myositis), myositis ossificans, rhabdomyolysis, and myoglobinurias, and disuse atrophy.

The present invention also provides therapeutic combinations that comprise a RANK antagonist antigen-binding molecule and at least one anti-ICM antagonist or at least one anti-AMA antagonist in methods for (1) treating a cancer, (2) delaying progression of a cancer, (3) inhibiting migration or metastasis of a cancer, (4) prolonging the survival of a patient suffering from a cancer, or (5) stimulating a cell mediated immune response to a cancer. Representative cancer include solid tumors, e.g., melanoma (e.g., an advanced stage (e.g., stage II-IV) melanoma or an HLA-A2 positive melanoma), pancreatic cancer (e.g., advanced pancreatic cancer), solid tumors, breast cancer (e.g., metastatic breast carcinoma, a breast cancer that does not express one, two or all of estrogen receptor, progesterone receptor, or Her2/neu, e.g., a triple negative breast cancer), renal cell carcinoma (e.g., advanced (e.g., stage IV) or metastatic renal cell carcinoma (MRCC)), prostate cancer (e.g., hormone refractory prostate adenocarcinoma), colon cancer, lung cancer (e.g., non-small cell lung cancer), bone cancer, skin cancer, cancer of the head or neck (e.g., HPV+squamous cell carcinoma), cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, Kaposi's sarcoma, epidermoid cancer, or squamous cell cancer, or hematological malignancies, e.g., Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia (e.g., relapsed or refractory chronic lymphocytic leukemia), solid tumors of childhood, lymphocytic lymphoma, multiple myeloma, myelodysplastic syndromes, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers. In some embodiments, the cancers are metastatic.

Specific concurrent and/or sequential dosing regimens for any given subject may be established based upon the specific disease or condition for which the patient has been diagnosed, or in conjunction with the stage of the patient's disease or condition. For example, if a patient is diagnosed with a less-aggressive cancer, or a cancer that is in its early stages, the patient may have an increased likelihood of achieving a clinical benefit and/or immune-related response to a concurrent administration of a RANK antagonist antigen-binding molecule and an anti-ICM or anti-AMA antigen-binding molecule. Alternatively, if a patient is diagnosed with a more-aggressive cancer, or a cancer that is in its later stages, the patient may have a decreased likelihood of achieving a clinical benefit and/or immune-related response to the concurrent administration, and thus may suggest that either higher doses of the RANK antagonist antigen-binding molecule and/or anti-ICM or anti-AMA antigen-binding molecule should be administered or more aggressive dosing regimens or either agent or combination therapy may be warranted.

A therapeutically or prophylactically effective amount of a RANK antagonist antigen-binding molecule either alone or in combination with an anti-ICM or anti-AMA antigen-binding molecule, will preferably be injected into the subject. The actual dosage employed can be varied depending upon the requirements of the patient and the severity of the condition being treated. Determination of the proper starting dosage for a particular situation is within the repertoire of a skilled person in the art, though the assignment of a treatment regimen will benefit from taking into consideration the indication and the stage of the disease. Nonetheless, it will be understood that the specific dose level and frequency of dosing for any particular subject can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred subjects for treatment include animals, most preferably mammalian species such as humans, and domestic animals such as dogs, cats, and the like, patient to cancer.

### 10. Kits

A further embodiment of the present invention is a kit for treating a cancer in a subject. This kit comprises any pharmaceutical composition as disclosed herein.

For use in the kits of the invention, pharmaceutical compositions comprising suitable therapeutic combinations, and optionally with instructions for cancer treatment. The kits may also include suitable storage containers (e.g., ampules, vials, tubes, etc.), for each pharmaceutical composition and other included reagents (e.g., buffers, balanced salt solutions, etc.), for use in administering the pharmaceutical compositions to subjects. The pharmaceutical compositions and other reagents may be present in the kits in any convenient form, such as, e.g., in a solution of in a powder pharmaceutical compositions. The kits may further include a packaging container, optionally having one or more partitions for housing the pharmaceutical composition and other optional reagents.

In order that the invention may be readily understood and put into practical effect, particular preferred embodiments will now be described by way of the following non-limiting examples.

### EXAMPLES

### Example 1

### Isolation of Antagonist Anti-Rank Antigen-Binding Molecule

A fully human Fab-based antibody phage display library was obtained from CSL (Parkville, Melbourne, Victoria, AUS). General procedures for construction and screening human Fab libraries were described in de Haard et al. (1998, Advanced Drug Delivery Reviews 31, 5-31; 1999, J. Biol. Chem. 274:18218-18230).

The library was screened for Fab fragments which bind to the entire recombinant extracellular region of the RANK protein, to facilitate the identification of Fabs targeting an epitope within the CDR2 and CDR3 regions, thus enabling the antagonism of RANKL and cross-reactive binding with mouse RANK.

The phagemid library was screened for binders to RANK using RANK-Fc protein immobilized on Dynabeads® M-280 Streptavidin (Invitrogen™, Thermo Fisher Scientific 11205D) by biotin-anti-human Fc antibody capture (Jackson ImmunoResearch Laboratories 109-065-098). The selections were carried out following methods described previously (Hoet et al., 2005. Nat Biotechnol. 23(3):344-348; Panousis et al., 2016. MAbs 8(3):436-453). Briefly, three rounds of selection were performed by incubating the streptavidin bead-depleted phage input with 10 μg of immobilized RANK-Fc in 2% milk/PBST (MTPBS, 0.1% Tween-20) for 20 minutes at room temperature and then washed 12 times. Prior to each round of panning, the phagemid library was depleted of non-specific binders to streptavidin and/or Fc by incubation with Dynabeads® M-280 Streptavidin and beads coated via biotin anti-human Fc antibody capture with an irrelevant human IgG antibody. Selected phage clones were amplified in log phase E. coli TG1 cells and the Fab-phagemid rescued by super-infection with M13K07 helper phage.

Soluble human RANKL was used to elute phage that bound to immobilized human RANK-Fc protein, in order to enrich for clones with RANK blocking potential.

Approximately one thousand individual clones were picked after the third round of selection and screened by Fab-phage ELISA for RANK binding. The Fab cDNA from the human RANK-Fc phagemid binders was sequenced, in order to determine unique clones. The variable heavy region of the Fab and light chains were PCR-amplified and sequenced essentially as described (Hoet et al., 2005, supra). The ELISA method employed was as per Panousis et al. (2016, supra).

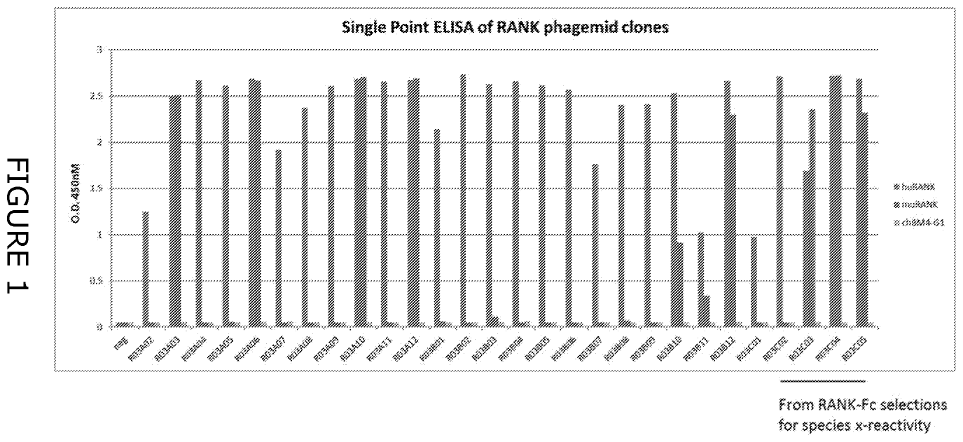

The unique positive huRANK-Fc binding phage clones isolated from RANKL elution experiments were then tested for species cross-reactivity to mouse RANK-Fc by phage ELISA. In summary: there were 5 phagemid clones (designated R03A03, R03A06, R03A10, R03A12, R03B12) which reacted to both human and mouse RANK-Fc by ELISA in addition to the 3 phagemid clones identified by alternating human RANK-Fc and mouse RANK-Fc binding (FIG. 1).

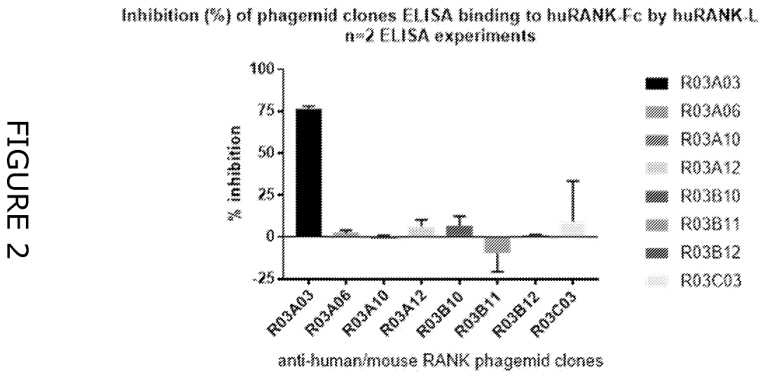

Unique phage clones which positively bound human RANK-Fc and mouse RANK-Fc were tested in a single-point phage competition human RANK-Fc ELISA with RANKL to determine whether phagemid clones may have RANKL/RANK blocking potential or antagonistic activity. Binding of one clone (R03A03) to human RANK-Fc was substantially (>75%) blocked in presence of human RANKL (FIG. 2). Binding of the other phagemid clones (R03A06, RO3A10, R03A12, R03B12, R03CO3, R03C04, R03C05) to human RANK-Fc were not blocked in presence of RANKL. Thus phagemid R03A03 has distinct properties from other RANK-binding phagemid clones.

The unique phagemid clones that bound to human RANK-Fc and also mouse RANK-Fc by ELISA were reformatted to full-length IgGs (human Fab on a mouse IgG2a Fc backbone). In total, twenty four unique antibody clones were reformatted to express full-length IgG (human Fab on a mouse IgG2a Fc backbone) antibodies. The human Fab is fused to the mouse IgG2aFc (without any linker sequence).

The IgGs were expressed from transient transfections and the purified protein was tested for binding by ELISA prior to functional in vitro potency testing. The IgGs were produced from transient transfection of suspension adapted 293T cells (Expi293F cells) using ExpiFectamine™ 293 transfection kit (Thermo Fisher Scientific) according to the manufacturer's instructions and as previously described (Spanevello et al., 2013, J Neurotrauma 30:1023-1034). Purification of the IgGs was performed as previously described in Panousis et al. (2016, supra).

### Example 2

### Antagonistic Activity of Anti-Rank Antibody in Cell-Based Functional Assay

To evaluate the functional inhibitory effect of the 3A3 antibody in a cell-based functional assay, the effect of this anti-RANK antibody on in vitro osteoclastogenesis was tested. The methods for the in vitro TRAP+ osteoclast assays were essentially as described (Simonet et al., 1997. Cell 89(2): 309-319). Bone marrow (BM) cells from normal BL/6 mice were seeded in a 96-well flat bottom plate at a density of 25000 cells/well in a total volume of 200 μL/well of complete DMEM (10% FCS+PS+Glu) supplemented with 50 ng/mL of human recombinant CSF-1 (Preprotech). After culture for 48 hr, media was replaced with complete DMEM supplemented with 50 ng/mL of human recombinant CSF-1 and 200 ng/mL of soluble muRANKL or soluble huRANKL (Miltenyi). Cells were cultured with CSF-1 and either human or mouse RANKL for 4 days (with and without antibody inhibitors) and then TRAP+ multinucleated (more than three nuclei) osteoclast cells were counted. The generated osteoclasts were evaluated by TRAP cytochemical staining as previously described (Simonet et al., 1997, supra).

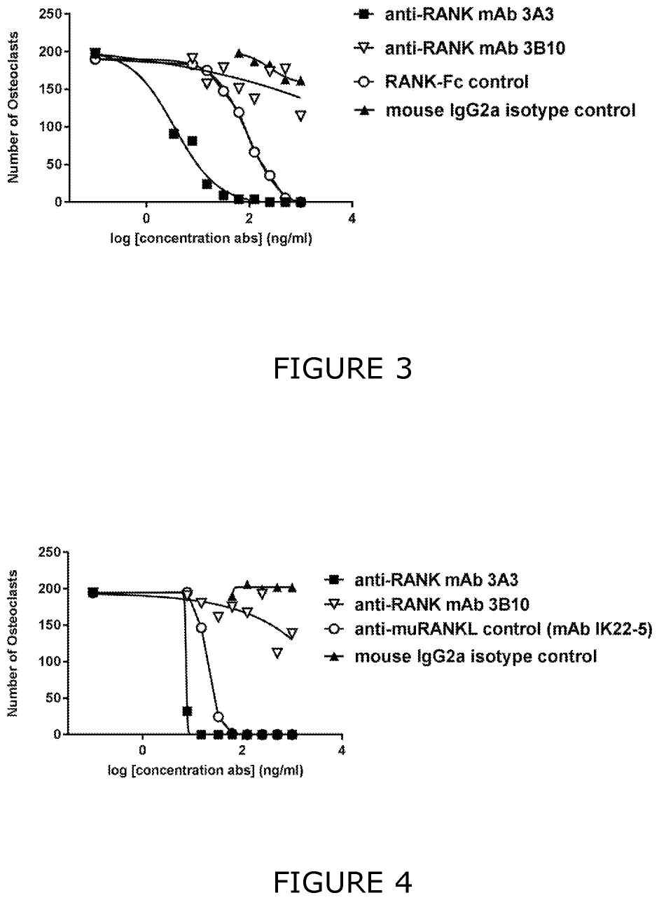

Osteoclast formation was assessed using recombinant human RANKL. Similar to the effect of the positive control RANK-Fc, the addition of the anti-RANK antibody 3A3, but not the addition of isotype IgG2a, inhibited the formation of TRAP+ multinucleated cells in a dose-dependent manner (FIG. 3). Notably, the addition of the non-blocking anti-RANK mAb 3610 did not affect osteoclast formation. The anti-RANK antibody 3A3 completely blocked osteoclast formation at concentrations between 125-250 ng/mL while the positive control RANK-Fc completely blocked osteoclast formation at concentrations between 500 ng/mL-1 μg/mL. The anti-RANK 3A3 antibody demonstrated antagonistic activity in blocking huRANKL-induced osteoclast formation with an IC₅₀ of 3.5 ng/mL; the control RANK-Fc had an IC₅₀ of 92 ng/mL.

These results demonstrate that the anti-RANK 3A3 antibody had at least equivalent activity compared with the positive control RANK-Fc in a cell based RANKL/RANK antagonistic assay (osteoclast formation). The calculated IC50s suggest that anti-RANK 3A3 antibody has approximately 25-fold greater potency compared with the positive control RANK-Fc. These results indicated that the anti-RANK 3A3 antibody retains an antagonistic activity against RANKL/RANK activity and the differentiation of osteoclasts in vitro.

In the next assay, osteoclast formation was assessed using recombinant mouse RANKL. Similar to the effect of the positive control anti-muRANKL IK22-5 mAb, the addition of the anti-RANK antibody 3A3, but not the addition of isotype IgG2a, inhibited the formation of TRAP+ multinucleated cells in a dose-dependent manner (FIG. 4). Again, the addition of the non-blocking anti-RANK mAb 3610 did not affect osteoclast formation. The anti-RANK antibody 3A3 completely blocked osteoclast formation at concentrations between 33-62.5 ng/mL while the positive control anti-muRANKL IK22-5 mAb completely blocked osteoclast formation at concentrations between 62.5-125 ng/mL. The anti-RANK 3A3 antibody demonstrated antagonistic activity in blocking muRANKL-induced osteoclast formation with an IC₅₀ of 7.4 ng/mL; the control anti-muRANKL mAb IK22-5 had an IC₅₀ of 19.8 ng/mL.

These results demonstrate that the anti-RANK 3A3 antibody had at least equivalent activity compared with the positive control anti-muRANKL mAb IK22-5 in a cell based RANKL/RANK antagonistic assay (osteoclast formation). The calculated IC50s suggest that anti-RANK 3A3 antibody has approximately 2-fold greater potency compared with the positive control anti-muRANKL mAb IK22-5. These results indicated that the anti-RANK 3A3 antibody retains an antagonistic activity against RANKL/RANK activity and the differentiation of osteoclasts in vitro.

### Example 3

### Dual Blockade of Rank and PD-L1 Significantly Enhances Fibrosarcoma Tumor Immunity

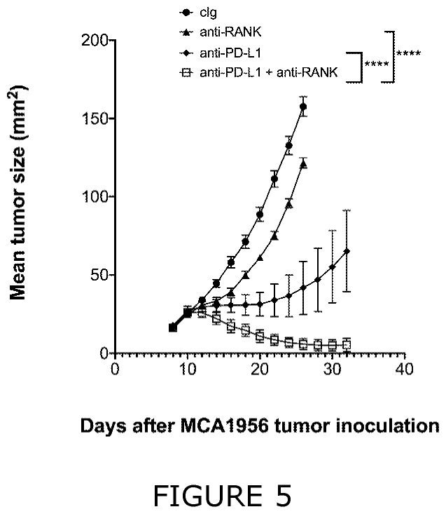

Given the results presented in Example 2, which demonstrate that the anti-RANK 3A3 antibody has at least equivalent activity compared with the positive control RANK-Fc in a cell based RANKL/RANK antagonistic assay (in vitro osteoclast formation), the efficacy of dual blockade of RANK and PD-L1 in mice bearing subcutaneous tumors was assessed using antagonistic anti-RANK and anti-PD-L1 antibodies. In the anti-PD-L1-sensitive MCA1956 fibrosarcoma model, the addition of antagonistic anti-RANK mAb showed a significantly enhanced anti-PD-L1 efficacy (FIG. 5, P<0.0001). This observation supports that the antagonistic anti-RANK 3A3 antibody may improve anti-tumor immunity.

### Example 4

### Dual Blockade of RANK and PD-L1 Significantly Enhances Colon Tumor Immunity

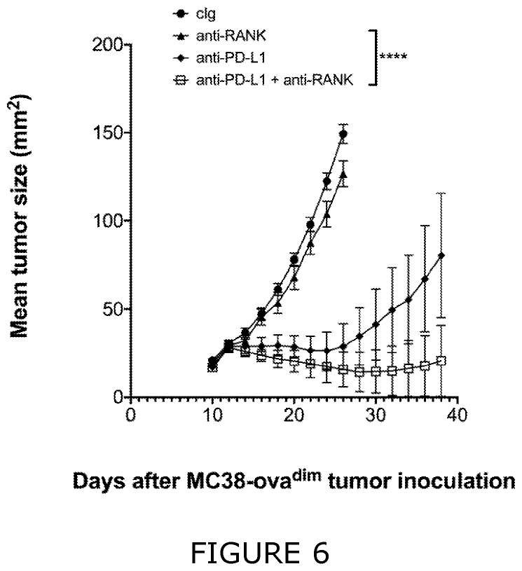

In the colon MC38 colon carcinoma model, the addition of antagonistic anti-RANK mAb showed a significantly enhanced anti-PD-L1 efficacy (FIG. 6, P<0.0001). This observation substantiates that the antagonistic anti-RANK 3A3 antibody may improve anti-tumor immunity.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A RANK antagonist antigen-binding molecule comprising:
(1) a heavy chain variable region (V_{H}) comprising a VHCDR1 amino acid sequence set forth in SEQ ID NO:3, a VHCDR2 amino acid sequence set forth in SEQ ID NO:4, and a VHCDR3 amino acid sequence set forth in SEQ ID NO:5, and a light chain variable region (V_{L}) comprising a VLCDR1 amino acid sequence set forth in SEQ ID NO:6, a VLCDR2 amino acid sequence set forth in SEQ ID NO:7, and a VLCDR3 amino acid sequence set forth in SEQ ID NO:8;
(2) a V_{H} that comprises the amino acid sequence set forth in SEQ ID NO:1, and a V_{L} that comprises the amino acid sequence set forth in SEQ ID NO:2;
(3) a V_{H} with at least 90% (including at least 91% to 99% and all integer percentages therebetween) sequence identity to the amino acid sequence of SEQ ID NO:1, and a V_{L} with at least 90% (including at least 91% to 99% and all integer percentages therebetween) sequence identity to the amino acid sequence of SEQ ID NO:2;
(4) a V_{H} with at least 90% (including at least 91% to 99% and all integer percentages therebetween) sequence identity to the amino acid sequence of a framework region other than each CDR in the amino acid sequence of SEQ ID NO:1, and a V_{L} with at least 90% (including at least 91% to 99% and all integer percentages therebetween) sequence identity to the amino acid sequence of a framework region other than each CDR in the amino acid sequence of SEQ ID NO:2; or
(5) a V_{H} that comprises an amino acid sequence comprising a deletion, substitution or addition of one or more (e.g., 1, 2, 3, 4 or 5) amino acids in the sequence of a framework region other than at each CDR in the amino acid sequence of SEQ ID NO:1, and a V_{L} that comprises an amino acid sequence comprising a deletion, substitution or addition of one or more (e.g., 1, 2, 3, 4 or 5) amino acids in the sequence of a framework region other than at each CDR in the amino acid sequence of SEQ ID NO:2.

2. (canceled)

3. (canceled)

4. (canceled)

5. (canceled)

6. An isolated polynucleotide comprising a nucleic acid sequence encoding the RANK antagonist antigen-binding molecule of claim 1.

7. (canceled)

8. (canceled)

9. (canceled)

10. A pharmaceutical composition comprising the RANK antagonist antigen-binding molecule of claim 1 and a pharmaceutically acceptable carrier.

11. The composition of claim 10, further comprising at least one ancillary agent selected from a bone anti-resorptive agent (e.g., anabolism enhancers, in particular selected from the group consisting of parathyroid hormone, BMP2, vitamin D, anti-inflammatory agents; and catabolism inhibitors, in particular selected from the group consisting of bisphosphonates, cathepsin K inhibitors, p38 inhibitors, JNK inhibitors, IKK inhibitors, NF-κB inhibitors, calcineurin inhibitors, NFAT inhibitors, PI3K inhibitors) and a chemotherapeutic agent (e.g., antiproliferative/antineoplastic drugs, cytostatic agents, agents that inhibit cancer cell invasion, inhibitors of growth factor function, anti-angiogenic agents, vascular damaging agents, etc.) or an immunotherapeutic agent (e.g., cytokines, cytokine-expressing cells, antibodies, etc.).

12. A method for (i) inhibiting binding of RANKL to a RANK-expressing cell, (ii) inhibiting activation of RANK on a RANK-expressing cell, (iii) inhibiting RANK-mediated molecular signaling (e.g., RANK recruitment of TRAF proteins) in a RANK-expressing cell, (iv) inhibiting RANK multimerization in a RANK-expressing cell, (v) inhibiting differentiation, activation and/or survival of an osteoclast, (vi) inhibiting immunosuppressive activity of an immune cell (e.g., a myeloid cell or Treg), (vii) inhibiting proliferation, survival or migration of a tumor cell; or (viii) treating or inhibiting the development of a condition associated with activation of the RANKL/RANK signaling pathway in a subject, the method comprising contacting the RANK-expressing cell with the RANK antagonist antigen-binding molecule of claim 1, to thereby (i) inhibit binding of RANK to the RANK expressing cell, (ii) inhibit activation of RANK on a RANK-expressing cell, (iii) inhibit RANK-mediated molecular signaling (e.g., RANK recruitment of TRAF proteins) in a RANK-expressing cell, (iv) inhibit RANK multimerization in a RANK-expressing cell, (v) inhibit differentiation, activation and/or survival of an osteoclast, (vi) inhibit immunosuppressive activity of an immune cell (e.g., a myeloid cell or Treg), (vii) inhibit proliferation, survival or migration of a tumor cell, or (viii) treat or inhibit the development of a condition associated with activation of the RANKL/RANK signaling pathway in a subject.

13. (canceled)

14. (canceled)

15. (canceled)

16. (canceled)

17. (canceled)

18. (canceled)

19. (canceled)

20. (canceled)

21. The method of claim 12, wherein the condition associated with RANKL/RANK signaling pathway activation is selected from an osteopenic disorder, a myopathy and a cancer.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A multispecific antigen-binding molecule for co-antagonizing RANK and at least one ICM, the multispecific antigen-binding molecules comprising, consisting or consisting essentially of the RANK antagonist antigen-binding molecule of claim 1 and at least one anti-ICM antigen-binding molecule.

55. (canceled)

56. (canceled)

57. (canceled)

58. (canceled)

59. (canceled)

60. (canceled)

61. (canceled)

62. The multispecific antigen-binding molecule of claim 54, wherein individual antigen-binding molecules are linked to or comprise a constant domain that is independently selected from the group consisting of IgG (e.g., IgG1, IgG2a, IgG2b, IgG3, or IgG4), IgM, IgD, IgA, and IgE.

63. The multispecific antigen-binding molecule of claim 54, which comprises a tandem scFv (taFv or scFv₂), diabody, dAb₂/VHH₂, knobs-in-holes derivative, Seedcod-IgG, heteroFc-scFv, Fab-scFv, scFv-Jun/Fos, Fab′-Jun/Fos, tribody, DNL-F(ab)₃, scFv₃-C_{H}1/C_{L}, Fab-scFv₂, IgG-scFab, IgG-scFv, scFv-IgG, scFv₂-Fc, F(ab′)₂-scFv₂, scDB-Fc, scDb-C_{H}3, db-Fc, scFv₂-H/L, DVD-Ig, tandAb, scFv-dhlx-scFv, dAb₂-IgG, dAb-IgG, dAb-Fc-dAb, tandAb, DART, BiKE, TriKE, mFc-V_{H}, crosslinked MAbs, Cross MAbs, MAb₂, FIT-Ig, electrostatically matched antibodies, symmetric IgG-like antibodies, LUZ-Y, Fab-exchanged antibodies, or a combination thereof.

64. (canceled)

65. The multispecific antigen-binding molecule of claim 62,
wherein the immunoglobulin constant chain comprises a light chain selected from a κ light chain and λ light chain, and/or a heavy chain selected from a γ1 heavy chain, γ2 heavy chain, γ3 heavy chain, and γ4 heavy chain.

66. The multispecific antigen-binding molecule of claim 54, wherein the multispecific antigen-binding molecule antagonizes PD-1, and the anti-PD-1 antigen-binding molecule (e.g., an antibody or antigen-binding fragment thereof) binds specifically to one or more amino acids of an amino acid sequence selected from SEQ ID NO:9 (i.e., residues 62 to 86 of the native human PD-1 sequence set forth in SEQ ID NO:10), SEQ ID NO:11 (i.e., residues 118 to 136 of the native human PD-1 sequence set forth in SEQ ID NO:10) and SEQ ID NO:12 (i.e., corresponding to residue 66 to 97 of the native human PD-1 sequence set forth in SEQ ID NO:10).

67. The multispecific antigen-binding molecule of claim 66, wherein the anti-PD-1 antigen-binding molecule (e.g., an antibody or antigen-binding fragment thereof) comprises a heavy chain and a light chain of a MAb selected from nivolumab, pembrolizumab, pidilizumab, and MEDI-0680 (AMP-514), AMP-224, JS001-PD-1, SHR-1210, Gendor PD-1, PDR001, CT-011, REGN2810, BGB-317 or antigen-binding fragments thereof.

68. The multispecific antigen-binding molecule of claim 54, wherein the multispecific antigen-binding molecule antagonizes PD-L1, and the anti-PD-L1 antigen-binding molecule (e.g., an antibody or antigen-binding fragment thereof) binds specifically to one or more amino acids of the amino acid sequence set forth in SEQ ID NO:13 (i.e., residues 279 to 290 of the native human PD-L1 amino acid sequence as set forth in SEQ ID NO:14).

69. (canceled)

70. The multispecific antigen-binding molecule of claim 54, wherein the multispecific antigen-binding molecule antagonizes CTLA4, and the anti-CTLA4 antigen-binding molecule (e.g., an antibody or antigen-binding fragment thereof) binds specifically to one or more amino acids of an amino acid sequence selected from SEQ ID NO:15 (i.e., residues 25 to 42 of the full-length native PD-CTLA4 amino acid sequence set forth in SEQ ID NO:16), SEQ ID NO:17 (i.e., residues 43 to 65 of the native CTLA4 sequence set forth in SEQ ID NO:16), and SEQ ID NO:18 (i.e., residues 96 to 109 of the native CTLA4 sequence set forth in SEQ ID NO:16).

71. (canceled)

72. The multispecific antigen-binding molecule of claim 54, further comprises, consists or consists essentially of an anti-PD-1 antigen-binding molecule.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A method for stimulating or augmenting immunity in a subject, or inhibiting the development or progression of immunosuppression or tolerance to a tumor in a subject, the method comprising, consisting or consisting essentially of administering to the subject an effective amount of the multispecific antigen-binding molecule of claim 54, to thereby stimulate or augment immunity in the subject, or inhibit the development or progression of immunosuppression or tolerance to a tumor in a subject.

87. (canceled)

88. (canceled)

89. (canceled)

90. (canceled)

91. (canceled)

92. (canceled)

93. (canceled)

94. (canceled)

95. (canceled)

96. (canceled)

97. A method for treating a cancer in a subject, the method comprising, consisting or consisting essentially of administering to the subject an effective amount of the multispecific antigen-binding molecule of claim 54, to thereby treat the cancer.

98. The method of claim 97, wherein the cancer is selected from melanoma, breast cancer, colon cancer, ovarian cancer, endometrial and uterine carcinoma, gastric or stomach cancer, pancreatic cancer, prostate cancer, salivary gland cancer, lung cancer, hepatocellular cancer, glioblastoma, cervical cancer, liver cancer, bladder cancer, hepatoma, rectal cancer, colorectal cancer, kidney cancer, vulval cancer, thyroid cancer, hepatic carcinoma, anal carcinoma, penile carcinoma, testicular cancer, esophageal cancer, tumors of the biliary tract, head and neck cancer, and squamous cell carcinoma. In some particular embodiments, the cancer is a metastatic cancer.

99. (canceled)

100. The method of claim 86, further comprising concurrently administering to the subject an effective amount of an ancillary anti-cancer agent, wherein the ancillary anti-cancer agent optionally includes a chemotherapeutic agent, external beam radiation, a targeted radioisotope, and a signal transduction inhibitor.

101. (canceled)

102. (canceled)


SEQUENCE LISTING (189 sequences)

SEQ ID NO: 1 - protein, 123 aa, Homo sapiens
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSS

SEQ ID NO: 2 - protein, 106 aa, Homo sapiens
SYELTQPPSVSVSPGQTASITCSGDKLGDKYVCWYQQKPGQSPVLVIYGDSERPSGIPERFSGSNSGNTATLTISGTRAVDEADYYCQAWDSTTPLFGGGTNLTVL

SEQ ID NO: 3 - protein, 10 aa, Homo sapiens
GFTFSSYAMH

SEQ ID NO: 4 - protein, 10 aa, Homo sapiens
VVSYDGSTKS

SEQ ID NO: 5 - protein, 14 aa, Homo sapiens
DPALRYFDWGYFQH

SEQ ID NO: 6 - protein, 11 aa, Homo sapiens
SGDKLGDKYVC

SEQ ID NO: 7-8 - skipped (2 entries)

SEQ ID NO: 9 - protein, 25 aa, Homo sapiens
SFVLNWYRMSPSNQTDKLAAFPEDR

SEQ ID NO: 10 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 11 - protein, 19 aa, Homo sapiens
SGTYLCGAISLAPKAQIKE

SEQ ID NO: 12 - protein, 32 aa, Homo sapiens
NWYRMSPSNQTDKLAAFPEDRSQPGQDCRFRV

SEQ ID NO: 13 - protein, 12 aa, Homo sapiens
SKKQSDTHLEET

SEQ ID NO: 14 - protein, 290 aa, Artificial Sequence
  Synthetic antigen binding molecule
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 18 aa, Homo sapiens
YASPGKATEVRVTVLRQA

SEQ ID NO: 16 - protein, 188 aa, Artificial Sequence
  Synthetic antigen binding molecule
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 17 - protein, 244 aa, Artificial Sequence
  Synthetic antigen binding molecule
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSSGGGGSGGGGSGGGGSSYELTQPPSVSVSPGQTASITCSGDKLGDKYVCWYQQKPGQSPVLVIYGDSERPSGIPERFSGSNSGNTATLTISGTRAVDEADYYCQAWDSTTPLFGGGTNLTVL

SEQ ID NO: 18 - protein, 15 aa, Artificial Sequence
  Synthetic antigen binding molecule
GGGGSGGGGSGGGGS

SEQ ID NO: 19 - protein, 453 aa, Artificial Sequence
  Synthetic antigen binding molecule
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 20 - protein, 213 aa, Artificial Sequence
  Synthetic antigen binding molecule
SYELTQPPSVSVSPGQTASITCSGDKLGDKYVCWYQQKPGQSPVLVIYGDSERPSGIPERFSGSNSGNTATLTISGTRAVDEADYYCQAWDSTTPLFGGGTNLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 222 aa, Artificial Sequence
  Synthetic antigen binding molecule
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 340 aa, Artificial Sequence
  Synthetic antigen binding molecule
EPKSCDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 23 - protein, 584 aa, Artificial Sequence
  Synthetic antigen binding molecule
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSSGGGGSGGGGSGGGGSSYELTQPPSVSVSPGQTASITCSGDKLGDKYVCWYQQKPGQSPVLVIYGDSERPSGIPERFSGSNSGNTATLTISGTRAVDEADYYCQAWDSTTPLFGGGTNLTVLEPKSCDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - skipped

SEQ ID NO: 25 - protein, 17 aa, murine
VIWYDGSKRYYADSVKG

SEQ ID NO: 26 - skipped

SEQ ID NO: 27 - protein, 11 aa, murine
RASQSVSSYLA

SEQ ID NO: 28-29 - skipped (2 entries)

SEQ ID NO: 30 - protein, 440 aa, Artificial Sequence
  Synthetic antigen binding molecule
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 113 aa, Artificial Sequence [sequence omitted]

SEQ ID NO: 32 - protein, 214 aa, Artificial Sequence
  Synthetic antigen binding molecule
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 107 aa, Artificial Sequence [sequence omitted]

SEQ ID NO: 34 - skipped

SEQ ID NO: 35 - protein, 17 aa, murine
GINPSNGGTNFNEKFKN

SEQ ID NO: 36 - protein, 11 aa, murine
RDYRFDMGFDY

SEQ ID NO: 37 - protein, 15 aa, murine
RASKGVSTSGYSYLH

SEQ ID NO: 38-39 - skipped (2 entries)

SEQ ID NO: 40 - protein, 447 aa, Artificial Sequence
  Synthetic antigen binding molecule
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 120 aa, Artificial Sequence [sequence omitted]

SEQ ID NO: 42 - protein, 218 aa, Artificial Sequence
  Synthetic antigen binding molecule
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 111 aa, Artificial Sequence [sequence omitted]

SEQ ID NO: 44 - skipped

SEQ ID NO: 45 - protein, 17 aa, murine
WINTDSGESTYAEEFKG

SEQ ID NO: 46 - skipped

SEQ ID NO: 47 - protein, 10 aa, murine
SARSSVSYMH

SEQ ID NO: 48-49 - skipped (2 entries)

SEQ ID NO: 50 - protein, 447 aa, Artificial Sequence
  Synthetic antigen binding molecule
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 117 aa, Artificial Sequence
  Synthetic antigen binding molecule
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 52 - protein, 213 aa, Artificial Sequence
  Synthetic antigen binding molecule
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 106 aa, Artificial Sequence
  Synthetic antigen binding molecule
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 54 - protein, 15 aa, murine
RASKSVSTSGFSYLH

SEQ ID NO: 55-57 - skipped (3 entries)

SEQ ID NO: 58 - protein, 17 aa, murine
GVNPSNGGTNFSEKFKS

SEQ ID NO: 59 - protein, 11 aa, murine
RDSNYDGGFDY

SEQ ID NO: 60 - protein, 15 aa, murine
RASKGVSTSGYSYLH

SEQ ID NO: 61-63 - skipped (3 entries)

SEQ ID NO: 64 - protein, 17 aa, murine
GINPSNGGTNFNEKFKN

SEQ ID NO: 65 - protein, 11 aa, murine
RDYRFDMGFDY

SEQ ID NO: 66 - protein, 120 aa, Artificial Sequence [sequence omitted]

SEQ ID NO: 67 - protein, 111 aa, Artificial Sequence [sequence omitted]

SEQ ID NO: 68 - protein, 110 aa, murine
IVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 69 - protein, 111 aa, Artificial Sequence
  Synthetic antigen binding molecule
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 70 - protein, 447 aa, Artificial Sequence; identical to SEQ ID NO: 40
  Synthetic antigen binding molecule

SEQ ID NO: 71 - protein, 218 aa, Artificial Sequence; identical to SEQ ID NO: 42
  Synthetic antigen binding molecule

SEQ ID NO: 72 - protein, 218 aa, Artificial Sequence
  Synthetic antigen binding molecule
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 218 aa, Artificial Sequence
  Synethic antigen binding molecule
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - skipped

SEQ ID NO: 75 - protein, 16 aa, murine
NIKQDGSEKYYVDSVK

SEQ ID NO: 76 - protein, 12 aa, murine
EGGWFGELAFDY

SEQ ID NO: 77 - protein, 12 aa, murine
RASQRVSSSYLA

SEQ ID NO: 78 - protein, 11 aa, murine
DASSRATGIPD

SEQ ID NO: 79 - skipped

SEQ ID NO: 80 - protein, 449 aa, Artificial Sequence
  Synthetic antigen binding molecule
VQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 120 aa, Artificial Sequence
  Synthetic antigen binding molecule
VQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 82 - protein, 215 aa, Artificial Sequence
  Synthetic antigen binding molecule
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 108 aa, Artificial Sequence
  Synthetic antigen binding molecule
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 84 - protein, 10 aa, Artificial Sequence
  Synthetic antigen binding molecule
  MISC_FEATURE (6)..(6): X is D or G
GFTFSXSWIH

SEQ ID NO: 85 - protein, 18 aa, Artificial Sequence
  Synthetic antigen binding molecule
  MISC_FEATURE (4)..(4): X is S or L
  MISC_FEATURE (10)..(10): X is T or S
AWIXPYGGSXYYADSVKG

SEQ ID NO: 86 - skipped

SEQ ID NO: 87 - protein, 11 aa, Artificial Sequence
  Synthetic antigen binding molecule
  MISC_FEATURE (5)..(5): X is D or V
  MISC_FEATURE (6)..(6): X is V or I
  MISC_FEATURE (7)..(7): X is S or N
  MISC_FEATURE (9)..(9): X is A or F
  MISC_FEATURE (10)..(10): X is V or L
RASQXXXTXXA

SEQ ID NO: 88-89 - skipped (2 entries)

SEQ ID NO: 90 - protein, 448 aa, Artificial Sequence
  Synthetic antigen binding molecule
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 118 aa, Artificial Sequence [sequence omitted]

SEQ ID NO: 92 - protein, 214 aa, Artificial Sequence
  Synthetic antigen binding molecule
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 107 aa, Artificial Sequence [sequence omitted]

SEQ ID NO: 94 - skipped

SEQ ID NO: 95 - protein, 17 aa, Artificial Sequence
  Synthetic antigen binding molecule
  MISC_FEATURE (8)..(8): X is F or I
  MISC_FEATURE (14)..(14): X is S or T
SIYPSGGXTFYADXVKG

SEQ ID NO: 96 - protein, 11 aa, Artificial Sequence
  Synthetic antigen binding molecule
  MISC_FEATURE (10)..(10): X is E or D
IKLGTVTTVXY

SEQ ID NO: 97 - protein, 14 aa, Artificial Sequence
  Synthetic antigen binding molecule
  MISC_FEATURE (4)..(4): X is N or S
  MISC_FEATURE (5)..(5): X is T, R, or S
  MISC_FEATURE (9)..(9): X is A or G
TGTXXDVGXYNYVS

SEQ ID NO: 98-99 - skipped (2 entries)

SEQ ID NO: 100 - protein, 450 aa, Artificial Sequence
  Synthetic antigen binding molecule
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 120 aa, Artificial Sequence
  Synthetic antigen binding molecule
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 102 - protein, 216 aa, Artificial Sequence
  Synthetic antigen binding molecule
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 103 - protein, 110 aa, Artificial Sequence
  Synthetic antigen binding molecule
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 104 - skipped

SEQ ID NO: 105 - protein, 17 aa, Homo sapiens
FISYDGNNKYYADSVKG

SEQ ID NO: 106 - skipped

SEQ ID NO: 107 - protein, 12 aa, Homo sapiens
RASQSVGSSYLA

SEQ ID NO: 108-109 - skipped (2 entries)

SEQ ID NO: 110 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 112 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 114 - protein, 10 aa, Homo sapiens
GFTFSSYGMH

SEQ ID NO: 115 - protein, 15 aa, Homo sapiens
VIWYDGSNKYYADSV

SEQ ID NO: 116 - protein, 16 aa, Homo sapiens
DPRGATLYYYYYGMDV

SEQ ID NO: 117 - protein, 11 aa, Homo sapiens
RASQSINSYLD

SEQ ID NO: 118-119 - skipped (2 entries)

SEQ ID NO: 120 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYIQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - protein, 118 aa, Homo sapiens; identical to SEQ ID NO: 111

SEQ ID NO: 122 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 107 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 124 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 125 - skipped

SEQ ID NO: 126 - protein, 16 aa, murine
YISSDSSAIYYADTVK

SEQ ID NO: 127 - protein, 13 aa, murine
GRENIYYGSRLDY

SEQ ID NO: 128 - protein, 11 aa, murine
KASQNVDTNVA

SEQ ID NO: 129-130 - skipped (2 entries)

SEQ ID NO: 131 - protein, 451 aa, Artificial Sequence
  Synthetic antigen binding molecule
VQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 132 - protein, 121 aa, Artificial Sequence
  Synthetic antigen binding molecule
VQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 133 - protein, 214 aa, Artificial Sequence
  Synthetic antigen binding molecule
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 107 aa, Artificial Sequence
  Synthetic antigen binding molecule
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 135 - protein, 327 aa, Homo sapiens
HEGVHRKPSLLAHPGPLVKSEETVILQCWSDVMFEHFLLHREGMFNDTLRLIGEHHDGVSKANFSISRMTQDLAGTYRCYGSVTHSPYQVSAPSDPLDIVIIGLYEKPSLSAQPGPTVLAGENVTLSCSSRSSYDMYHLSREGEAHERRLPAGPKVNGTFQADFPLGPATHGGTYRCFGSFHDSPYEWSKSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 136 - skipped

SEQ ID NO: 137 - protein, 16 aa, murine
GFIPIFGAANYAQKFQ

SEQ ID NO: 138 - protein, 14 aa, murine
IPSGSYYYDYDMDV

SEQ ID NO: 139 - protein, 11 aa, murine
RASQSVSSYLA

SEQ ID NO: 140-141 - skipped (2 entries)

SEQ ID NO: 142 - protein, 450 aa, Artificial Sequence
  Synthetic antigen binding molecule
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 123 aa, Artificial Sequence
  Synthetic antigen binding molecule
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSS

SEQ ID NO: 144 - protein, 214 aa, Artificial Sequence
  Synthetic antigen binding molecule
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 109 aa, Artificial Sequence
  Synthetic antigen binding molecule
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 146 - protein, 503 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 147 - skipped

SEQ ID NO: 148 - protein, 16 aa, murine
EINHRGSTNSNPSLKS

SEQ ID NO: 149 - protein, 12 aa, murine
GYSDYEYNWFDP

SEQ ID NO: 150 - protein, 11 aa, murine
RASQSISSYLA

SEQ ID NO: 151-152 - skipped (2 entries)

SEQ ID NO: 153 - protein, 447 aa, Artificial Sequence
  Synthetic antigen binding molecule
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 154 - protein, 120 aa, Artificial Sequence
  Synthetic antigen binding molecule
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 155 - protein, 18 aa, Artificial Sequence
  Synthetic antigen binding molecule
GGSSRSSSSGGGGSGGGG

SEQ ID NO: 156 - protein, 214 aa, Artificial Sequence
  Synthetic antigen binding molecule
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 107 aa, Artificial Sequence
  Synthetic antigen binding molecule
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 158 - protein, 123 aa, Artificial Sequence; identical to SEQ ID NO: 1
  Synthetic antigen binding molecule

SEQ ID NO: 159 - protein, 107 aa, Artificial Sequence [sequence omitted]

SEQ ID NO: 160 - protein, 113 aa, Artificial Sequence [sequence omitted]

SEQ ID NO: 161 - protein, 106 aa, Artificial Sequence; identical to SEQ ID NO: 2
  Synthetic antigen binding molecule

SEQ ID NO: 162 - protein, 123 aa, Artificial Sequence; identical to SEQ ID NO: 1
  Synthetic antigen binding molecule

SEQ ID NO: 163 - protein, 108 aa, Artificial Sequence; identical to SEQ ID NO: 83
  Synthetic antigen binding molecule

SEQ ID NO: 164 - protein, 120 aa, Artificial Sequence; identical to SEQ ID NO: 81
  Synthetic antigen binding molecule

SEQ ID NO: 165 - protein, 106 aa, Artificial Sequence; identical to SEQ ID NO: 2
  Synthetic antigen binding molecule

SEQ ID NO: 166 - protein, 123 aa, Artificial Sequence; identical to SEQ ID NO: 1
  Synthetic antigen binding molecule

SEQ ID NO: 167 - protein, 107 aa, Artificial Sequence [sequence omitted]

SEQ ID NO: 168 - protein, 118 aa, Artificial Sequence [sequence omitted]

SEQ ID NO: 169 - protein, 106 aa, Artificial Sequence; identical to SEQ ID NO: 2
  Synthetic antigen binding molecule

SEQ ID NO: 170 - protein, 123 aa, Artificial Sequence; identical to SEQ ID NO: 1
  Synthetic antigen binding molecule

SEQ ID NO: 171 - protein, 108 aa, Artificial Sequence; identical to SEQ ID NO: 113
  Synthetic antigen binding molecule

SEQ ID NO: 172 - protein, 118 aa, Artificial Sequence; identical to SEQ ID NO: 111
  Synthetic antigen binding molecule

SEQ ID NO: 173 - protein, 106 aa, Artificial Sequence; identical to SEQ ID NO: 2
  Synthetic antigen binding molecule

SEQ ID NO: 174 - protein, 123 aa, Artificial Sequence; identical to SEQ ID NO: 1
  Synthetic antigen binding molecule

SEQ ID NO: 175 - protein, 107 aa, Artificial Sequence; identical to SEQ ID NO: 123
  Synthetic antigen binding molecule

SEQ ID NO: 176 - protein, 118 aa, Artificial Sequence; identical to SEQ ID NO: 111
  Synthetic antigen binding molecule

SEQ ID NO: 177 - protein, 106 aa, Artificial Sequence; identical to SEQ ID NO: 2
  Synthetic antigen binding molecule

SEQ ID NO: 178 - protein, 221 aa, Artificial Sequence
  Synthetic antigen binding molecule
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 179 - protein, 212 aa, Artificial Sequence
  Synthetic antigen binding molecule
SYELTQPPSVSVSPGQTASITCSGDKLGDKYVCWYQQKPGQSPVLVIYGDSERPSGIPERFSGSNSGNTATLTISGTRAVDEADYYCQAWDSTTPLFGGGTNLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 180 - protein, 461 aa, Artificial Sequence
  Synthetic antigen binding molecule
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSSGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 204 aa, Artificial Sequence
  Synthetic antigen binding molecule
SYELTQPPSVSVSPGQTASITCSGDKLGDKYVCWYQQKPGQSPVLVIYGDSERPSGIPERFSGSNSGNTATLTISGTRAVDEADYYCQAWDSTTPLFGGGTNLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 182 - protein, 448 aa, Artificial Sequence
  Synthetic antigen binding molecule
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 183 - protein, 214 aa, Artificial Sequence; identical to SEQ ID NO: 92
  Synthetic antigen binding molecule

SEQ ID NO: 184 - protein, 458 aa, Artificial Sequence
  Synthetic antigen binding molecule
EVQLVESGGGVVQPGTSLRLSCAASGFTFSSYAMHWVRQAPGKGLEWVAVVSYDGSTKSYADSMKGRFTISRDNSKNTLYLQMNSLRAEDTAVYYCAKDPALRYFDWGYFQHWGQGTLVTVSSGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - protein, 204 aa, Artificial Sequence; identical to SEQ ID NO: 181
  Synthetic antigen binding molecule

SEQ ID NO: 186 - protein, 440 aa, Artificial Sequence
  Synthetic antigen binding molecule
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLASRLTVDKSRWQEGNVFSCSVMHEALHNHYTQKSLSLSLGK

SEQ ID NO: 187 - protein, 214 aa, Artificial Sequence; identical to SEQ ID NO: 32
  Synthetic antigen binding molecule

SEQ ID NO: 188 - protein, 23 aa, Homo sapiens
DSQVTEVCAATYMMGNELTFLDD

SEQ ID NO: 189 - protein, 14 aa, Homo sapiens
VELMYPPPYYLGIG